SEQUENCE LISTING (116 sequences)

SEQ ID NO: 1 - protein, 332 aa, synthetic construct
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIG

SEQ ID NO: 2 - protein, 96 aa, synthetic construct
CSGHIWVEPATIFKMGMNISIYCQAAIKNCQPRKLHFYKNGIKERFQITRINKTTARLWYKNFLEPHASMYCTAECPKHFQETLICGKDISSGYPP

SEQ ID NO: 3 - protein, 90 aa, synthetic construct
DIPDEVTCVIYEYSGNMTCTWNAGKLTYIDTKYVVHVKSLETEEEQQYLTSSYINISTDSLQGGKKYLVWVQAANALGMEESKQLQIHLD

SEQ ID NO: 4 - protein, 103 aa, synthetic construct
DIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPET

SEQ ID NO: 5 - protein, 192 aa, synthetic construct
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLD

SEQ ID NO: 6 - protein, 295 aa, synthetic construct
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPET

SEQ ID NO: 7 - protein, 522 aa, synthetic construct
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSD

SEQ ID NO: 8 - protein, 91 aa, synthetic construct
GPRDLRCYRISSDRYECSWQYEGPTAGVSHFLRCCLSSGRCCYFAAGSATRLQFSDQAGVSVLYTVTLWVESWARNQTEKSPEVTLQLYNS

SEQ ID NO: 9 - protein, 93 aa, synthetic construct
PLGDIKVSKLAGQLRMEWETPDNQVGAEVQFRHRTPSSPWKLGDCGPQDDDTESCLCPLEMNVAQEFQLRRRQLGSQGSSWSKWSSPVCVPPE

SEQ ID NO: 10 - protein, 184 aa, synthetic construct
GPRDLRCYRISSDRYECSWQYEGPTAGVSHFLRCCLSSGRCCYFAAGSATRLQFSDQAGVSVLYTVTLWVESWARNQTEKSPEVTLQLYNSPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPE

SEQ ID NO: 11 - protein, 449 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 213 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13-44 - skipped (32 entries)

SEQ ID NO: 45 - protein, 355 aa, synthetic construct
WQPDTAHHWATLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGITNINCSGHIWVEPATIFKMGMNISIYCQAAIKNCQPRKLHFYKNGIKERFQITRINKTTARLWYKNFLEPHASMYCTAECPKHFQETLICGKDISSGYPP

SEQ ID NO: 46 - protein, 453 aa, synthetic construct
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 214 aa, synthetic construct
DIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48-51 - skipped (4 entries)

SEQ ID NO: 52 - protein, 241 aa, synthetic construct
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSWQPDTAHHWATL

SEQ ID NO: 53 - protein, 612 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKIQIKESLRAELRVTERRAEVPTAHPSPSPRPAGQFQTLV

SEQ ID NO: 54 - protein, 217 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 55 - protein, 15 aa, synthetic construct
GGGGSGGGGSGGGGS

SEQ ID NO: 56 - protein, 150 aa, synthetic construct [sequence omitted]

SEQ ID NO: 57 - protein, 150 aa, synthetic construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKIQIKESLRAELRVTERRAEVPTAHPSPSPRPAGQFQTLV

SEQ ID NO: 58 - protein, 137 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 59 - protein, 732 aa, synthetic construct
SKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLE

SEQ ID NO: 60 - protein, 745 aa, synthetic construct
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 61 - protein, 205 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 62 - protein, 120 aa, synthetic construct
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMAS

SEQ ID NO: 63 - protein, 181 aa, synthetic construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 64 - protein, 214 aa, synthetic construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 65 - protein, 167 aa, synthetic construct
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 66 - protein, 208 aa, synthetic construct [sequence omitted]

SEQ ID NO: 67 - protein, 224 aa, synthetic construct
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 68 - protein, 119 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 69 - protein, 172 aa, synthetic construct
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 70 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 71 - protein, 133 aa, synthetic construct
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 72 - protein, 128 aa, synthetic construct
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 73 - protein, 240 aa, synthetic construct
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWS

SEQ ID NO: 74 - protein, 146 aa, synthetic construct
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 75 - protein, 235 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 76 - protein, 183 aa, synthetic construct
IAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLP

SEQ ID NO: 77 - protein, 177 aa, synthetic construct
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 78 - protein, 263 aa, synthetic construct
MMPKHCFLGFLISFFLTGVAGTQSTHESLKPQRVQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 79 - protein, 120 aa, synthetic construct
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSS

SEQ ID NO: 80 - protein, 107 aa, synthetic construct
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIK

SEQ ID NO: 81 - protein, 117 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 82 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 83 - protein, 116 aa, synthetic construct [sequence omitted]

SEQ ID NO: 84 - protein, 107 aa, synthetic construct [sequence omitted]

SEQ ID NO: 85 - protein, 124 aa, synthetic construct
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSS

SEQ ID NO: 86 - protein, 111 aa, synthetic construct
QSVLTQPPSVSGAPGQRVTISCTGSSSNTGAGYDVHWYQQVPGTAPKLLIYGSGNRPSGVPDRFSGSKSGTSASLAITGLQAEDEADYYCQSYDSSLSGWVFGGGTRLTVL

SEQ ID NO: 87 - protein, 115 aa, synthetic construct
QVQLVQSGAEVKKPGSSVKVSCKASGYKFTRYVMHWVRQAPGQGLEWMGYINPYNDGTNYNEKFKGRVTITADKSTSTAYMELSSLRSEDTAVYYCARNWDTGLWGQGTTVTVSS

SEQ ID NO: 88 - protein, 107 aa, synthetic construct
DIQMTQSPSSLSASVGDRVTITCKASDHILKFLTWYQQKPGKAPKLLIYGATSLETGVPSRFSGSGSGTDFTLTISSLQPEDFATYYCQMYWSTPFTFGGGTKVEIK

SEQ ID NO: 89 - protein, 209 aa, synthetic construct
GNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 90-109 - skipped (20 entries)

SEQ ID NO: 110 - protein, 447 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 151 aa, synthetic construct
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQ

SEQ ID NO: 116 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESYGTEWSSYKTTPPVLDSDGSFFLYSKLTVTKEEWQQGFVFSCSVMHEALHNHYTQKSLSLSPGK

(19) United States
(12) Patent Application Publication
(10) Pub. No.: US 2024/0425579 A1
(43) Pub. Date: Dec. 26, 2024

(54) COMPOSITIONS AND METHODS THAT INHIBIT IL-23 SIGNALING

(71) Applicant: Y-TRAP, INC., South San Francisco, CA (US)

(72) Inventor: Rishi Bedi, San Francisco, CA (US)

(21) Appl. No.: 18/700,540

(22) PCT Filed: Oct. 11, 2022
(86) PCT No.: PCT/US2022/046287

§ 371 Date: Apr. 11, 2024

Related U.S. Application Data
(60) Provisional application No. 63/254,387, filed on Oct. 11, 2021.

(51) Int. Cl. C07K 16/24; A61K 45/06; A61P 35/00; C07K 16/30; C12N 15/62

(52) U.S. Cl. CPC C07K 16/244; A61K 45/06; A61P 35/00; C07K 16/30; C12N 15/62; C07K 2317/565; C07K 2317/76; C07K 2319/33

(57) ABSTRACT

The present disclosure provides recombinant molecules, compositions and methods for modulating the IL-23 axis. In certain embodiments, these recombinant molecules are used in methods of treating and/or preventing treatment of cancer, autoimmune diseases, and inflammatory disorders associated with the IL-23 axis and signaling.

### CROSS-REFERENCE TO RELATED APPLICATIONS

This application claims the benefit of and priority to U.S. Provisional Application No. 63/254,387 filed Oct. 11, 2021, which is incorporated by reference in its entirety.

### SEQUENCE LISTING

The instant application contains a Sequence Listing which has been submitted via Patent Center and is hereby incorporated by reference in its entirety. Said XML copy, created on XXX XX, 2022, is named 50048WO_sequencelisting.xml, and is XX,XXX bytes in size.

### BACKGROUND

Interleukin 23 (IL-23) is known to be a key pro-inflammatory cytokine in the development of chronic inflammatory diseases, such as psoriasis, inflammatory bowel diseases, multiple sclerosis, or rheumatoid arthritis. The pathological consequences of excessive IL-23 signaling have been linked to its ability to promote the production of inflammatory mediators, such as IL-17, IL-22, granulocyte-macrophage colony-stimulating (GM-CSF), or the tumor necrosis factor (TNFα) by target populations, mainly Th17 and IL-17-secreting TCRγδ cells (Tγδ17). IL-23 is also an important determinant of tumor-promoting pro-inflammatory signaling and the failure of the adaptive antitumor immunity.

Thus, there is a need for molecules that can modulate IL-23 signaling.

### SUMMARY

In one aspect, provided herein is a recombinant molecule comprising: (a) an interleukin-23 (IL-23) inhibiting polypeptide (IIP), wherein the IIP comprises IL-23 binding polypeptide or an IL-23R binding polypeptide; and (b) a target binding polypeptide moiety that binds one or more immune checkpoint proteins or immune stimulatory receptors.

In various embodiments, the target binding polypeptide binds an immune checkpoint protein as an antagonist or as an agonist. In various embodiments, the immune checkpoint protein is a T cell co-inhibitory receptor or ligand or an innate inhibitory receptor or ligand.

In some embodiments, the immune checkpoint protein is selected from programmed death-1 (PD1; CD279), programmed death ligand 1 (PDL1), programmed death ligand 2 (PDL2), cytotoxic T-lymphocyte antigen-4 (CTLA4; CD152), B and T lymphocyte attenuator (BTLA), V-domain immunoglobulin suppressor of T cell activation (VISTA), T cell immunoglobulin and ITIM domain (TIGIT), lymphocyte-activation gene 3 (LAG-3; CD223), T-cell immunoglobulin and mucin domain 3 (Tim-3; HAVCR2), carcinoembryonic antigen-related cell-adhesion molecule 1 (CEACAM1), CD47, signal regulatory protein alpha (SIRPa), Major Histocompatibility Complex, Class I, G (HLA-G), Ig-like transcript 2 (ILT2; LILRB1), or Ig-like transcript 4 (ILT4, LILRB2).

In some embodiments, the immune stimulatory receptor is selected from 4-1BB (CD137), Inducible T-cell costimulator (ICOS; CD278), OX-40 (CD134), glucocorticoid-induced TNFR-related protein (GITR; CD357), CD40, Herpesvirus entry mediator (HVEM), CD28, or CD27.

In some embodiments, the IIP binds and inhibits IL-23. In some embodiments, the IIP binds and inhibits the IL-23p19 subunit. In some embodiments, the IIP binds and inhibits IL-23R.

In various embodiments, the IIP comprises an antibody or an antigen binding fragment thereof. In some embodiments, the IIP is an antibody or an antigen binding fragment thereof, wherein the antigen binding fragment thereof comprises a fragment crystallizable (Fc) region, a fragment antigen binding (Fab) region, a single chain variable fragment (scFv), a light chain or a functional portion thereof, a variable region of the light chain (VL), a constant region of the light chain (CL), a heavy chain or a functional portion thereof, a variable region of the heavy chain (VH), a constant region of the heavy chain (CH), at least one complementarity-determining region (CDR) or a portion thereof, or any combination thereof.

In some embodiments, the antibody or an antigen binding fragment thereof comprises a monoclonal antibody that targets the human IL-23p19 subunit. In some embodiments, the antibody or antigen binding fragment thereof comprises one or more of the six CDRs or an antigen binding portion thereof selected from any one of risankizumab (VH: SEQ ID NO: 79; VL: SEQ ID NO: 80), guselkumab (VH: SEQ ID NO: 81; VL: SEQ ID NO: 82), tildrakizumab (VH: SEQ ID NO: 83; VL: SEQ ID NO: 84), brazikumab (VH: SEQ ID NO: 85; VL: SEQ ID NO: 86), and mirikizumab (VH: SEQ ID NO: 87; VL: SEQ ID NO: 88). In some embodiments, the antibody or antigen binding fragment thereof is guselkumab.

In some embodiments, the target binding polypeptide comprises an antibody or antigen binding fragment thereof. In some embodiments, the target binding polypeptide is an antibody or an antigen binding fragment thereof, wherein the antigen binding fragment thereof comprises a fragment crystallizable (Fc) region, a fragment antigen binding (Fab) region, a single chain variable fragment (scFv), a light chain or a functional portion thereof, a variable region of the light chain (VL), a constant region of the light chain (CL), a heavy chain or a functional portion thereof, a variable region of the heavy chain (VH), a constant region of the heavy chain (CH), at least one complementarity-determining region (CDR) or a portion thereof, or any combination thereof.

In some embodiments, the target binding polypeptide binds an immune checkpoint protein as an antagonist, wherein the target binding polypeptide comprises a ligand-binding sequence of the extracellular domain (ECD) of an immune checkpoint protein. In some embodiments, the ECD of the immune checkpoint protein is capable of specifically binding one or more of its cognate ligands expressed or displayed on a tumor cell or immune cell. In some embodiments, the immune cell is an antigen presenting cell (APC), myeloid-derived suppressor cell (MDSC), CD4 T cell, or T_{H}17 cell.

In some embodiments, the ECD is capable of specifically binding programmed death-1 ligand 1 (PDL1; CD274; B7-H1) and/or programmed death-1 ligand 2 (PDL2). In some embodiments, the target binding polypeptide comprises the PD1 (CD279) extracellular domain (PD1-ECD) or ligand-binding fragment thereof.

In some embodiments, the target binding polypeptide comprises the amino acid sequence of SEQ ID NO: 56, or an amino acid sequence having at least 80% identity to SEQ ID NO: 56 or a ligand-binding fragment thereof.

In some embodiments, the target binding polypeptide comprises one or more modifications of the amino acid sequence of SEQ ID NO: 56 or a ligand-binding fragment thereof, wherein the target binding polypeptide comprises substitution, deletion, insertion, or inversion of 1-10 amino acid residues. In some embodiments, the one or more modifications increase the affinity of the target binding polypeptide to PDL1 or PDL2 or both, compared to the affinity of wild type PD1-ECD to its ligands. In some embodiments, the one or more modifications are selected from A132I, S87G, P89L, N116S, G124S, S127V, A140V. In some embodiments, the modification is A132I. In some embodiments, the target binding polypeptide has the amino acid sequence of SEQ ID NO: 57.

In various embodiments, the IIP is linked to the target binding polypeptide moiety via a linker. In some embodiments, the target binding polypeptide moiety is linked to the C terminus of the IIP. In some embodiments, the linker is selected from a non-cleavable linker, a peptide linker, a flexible linker, a rigid linker, a helical linker, and a non-helical linker. In some embodiments, the linker is a peptide linker having an amino acid sequence comprising (GGGGS)n, wherein n is 1, 2, 3, 4, 5, 6, 7, or 8. In some embodiments, the linker comprises the amino acid sequence of SEQ ID NO: 55. In some embodiments, the recombinant molecule comprises a first polypeptide having an amino acid sequence of SEQ ID NO: 53 and a second polypeptide having an amino acid sequence of SEQ ID NO: 54.

In some aspects, provided herein is a host comprising the recombinant molecule as described herein.

In some aspects, provided herein is a polynucleotide sequence encoding the recombinant molecule as described herein. In some aspects, provided herein is a vector comprising the polynucleotide.

In some aspects, provided herein is a polypeptide comprising the recombinant molecule as described herein.

In some aspects, provided herein is a pharmaceutical composition comprising the recombinant molecule as described herein or a vector comprising the polynucleotide sequence encoding the recombinant molecule as described herein. In some embodiments, the pharmaceutical composition further comprises a pharmaceutically acceptable excipient.

In some aspects, provided herein is a method of treating a neoplastic disease, a cancer, or an immune disorder in a subject, comprising administering to the subject an effective amount of a pharmaceutical composition comprising a recombinant molecule as described herein or a vector comprising the polynucleotide sequence encoding the recombinant molecule as described herein.

In some embodiments, the subject has cancer. In some embodiments, the recombinant molecule comprises a first polypeptide having an amino acid sequence of SEQ ID NO: 53 and a second polypeptide having an amino acid sequence of SEQ ID NO: 54.

In some embodiments, the cancer is selected from prostate cancer, pancreatic cancer, biliary cancer, colon cancer, rectal cancer, liver cancer, kidney cancer, lung cancer, testicular cancer, breast cancer, ovarian cancer, brain cancer, skin cancer, bladder cancer, and head and neck cancers, melanoma, sarcoma, multiple myeloma, leukemia, and/or lymphoma.

In some embodiments, the method suppresses tumor growth for at least 10, 15, or 20 days. In some embodiments, the method reduces tumor growth by at least 5%, 10%, 15%, or 20%.

In some embodiments, the method further comprises administering to the subject a therapeutic agent comprising an anti-CTLA4-TGFβRII molecule. In some embodiments, the method further comprising administering one or more anti-cancer agents.

In some embodiments, the one or more anti-cancer agents comprises an immunotherapeutic agent, chemotherapeutic molecule, antibody, antibody-drug conjugate, small molecule kinase inhibitor, hormonal agent, androgen synthesis inhibitor, androgen receptor antagonist, anti-angiogenic agent, cell therapy, CAR-T cellular therapy, CAR-NK cellular therapy, radionuclide therapy, ionizing radiation, ultraviolet radiation, cryoablation, thermal ablation, a selective estrogen receptor modulator (SERM), a selective estrogen receptor degrader (SERD), or radiofrequency ablation. In some embodiments, the immunotherapeutic agent is selected from immune checkpoint inhibitor, immune stimulatory receptor agonist, immune stimulatory cytokine/cytokine receptor agonist, immune inhibitory cytokine/cytokine receptor antagonist, tumor vaccine, immunomodulatory imide drug, CAR-T cells, CAR-NK cells, or oncolytic virus.

In some embodiments, the administration of the anti-IL-23 agent in combination with the one or more anti-cancer agents reduces or prevents severe immune-related adverse events or toxicity more effectively compared to administration of the one or more anti-cancer agents alone. In some embodiments, the administration of the anti-IL-23 agent in combination with the one or more anti-cancer agents enhances reduction of tumor growth or suppresses tumor growth more effectively compared to administration of the one or more anti-cancer agents alone.

In some embodiments, the subject is a mammal. In some embodiments, the subject is a human.

In one aspect, provided herein is a method of treating a neoplastic disease or a cancer in a subject, comprising administering to the subject one or more therapeutic agents, wherein the one or more therapeutic agents comprises: (a) a first therapeutic agent comprising an inhibitor of IL-23 signaling; and (b) a second therapeutic agent comprising: (i) one or more modulators wherein each is an antagonist of one or more immune checkpoint proteins; (ii) one or more modulators wherein each is an agonist of one or more immune stimulatory receptors; (iii) one or more modulators wherein each is an antagonist of the signaling of one or more cytokines; (iv) one or more modulators wherein each is an agonist of one or more cytokine receptors; (v) one or more modulators wherein each modulates one or more cell surface molecules expressed or displayed on the cell surface of a tumor cell or an immune cell; (vi) one or more immune cells comprising CAR-T cells, CAR-NK cells, or hematopoietic stem cells; (vii) one or more immunogenic chemotherapeutic agents; and/or (viii) one or modulators wherein each is an antagonist of one or more immune inhibitory enzymes.

In some embodiments, the inhibitor of IL-23 signaling comprises an IL-23 binding moiety that is a recombinant protein that binds IL-23. In some embodiments, the inhibitor of IL-23 signaling comprises an IL-23R binding moiety that is a recombinant protein that binds IL-23R. In some embodiments, the inhibitor of IL-23 signaling comprises a recombinant molecule as described herein.

In various embodiments, the inhibitor of IL-23 signaling is an antibody or an antigen binding fragment thereof, wherein the antigen binding fragment thereof comprises a fragment crystallizable (Fc) region, a fragment antigen binding (Fab) region, a single chain variable fragment (scFv), a light chain or a functional portion thereof, a variable region of the light chain (VL), a constant region of the light chain (CL), a heavy chain or a functional portion thereof, a variable region of the heavy chain (VH), a constant region of the heavy chain (CH), at least one complementarity-determining region (CDR) or an antigen-binding portion thereof, or combinations thereof.

In some embodiments, the antibody or an antigen binding fragment thereof comprises a monoclonal antibody targeting an IL-23 subunit. In some embodiments, the monoclonal antibody targets the IL-23p19 subunit. In some embodiments, the antibody or an antigen binding fragment thereof comprises one or more of the six CDRs or an antigen binding portion thereof selected from any one of risankizumab, guselkumab, tildrakizumab, brazikumab, and mirikizumab.

In some embodiments, the IL-23R binding moiety comprises an antibody or antigen binding fragment thereof. In some embodiments, the antibody or antigen binding fragment thereof comprises one or more of the six CDRs or an antigen binding portion thereof of AS2762900-00.

In various embodiments, the second therapeutic agent comprises a modulator that is an antagonist of one or more immune checkpoint proteins. In some embodiments, the immune checkpoint protein is selected from programmed death-1 (PD1; CD279), programmed death ligand 1 (PDL1), programmed death ligand 2 (PDL2), cytotoxic T-lymphocyte antigen-4 (CTLA4; CD152), B and T lymphocyte attenuator (BTLA), V-domain immunoglobulin suppressor of T cell activation (VISTA), T cell immunoglobulin and ITIM domain (TIGIT), lymphocyte-activation gene 3 (LAG-3; CD223), T-cell immunoglobulin and mucin domain 3 (Tim-3; HAVCR2), carcinoembryonic antigen-related cell-adhesion molecule 1 (CEACAM1), CD47, signal regulatory protein alpha (SIRPa), Major Histocompatibility Complex, Class I, G (HLA-G), Ig-like transcript 2 (ILT2; LILRB1), or Ig-like transcript 4 (ILT4, LILRB2).

In some embodiments, the second therapeutic agent comprises a modulator that is an antagonist of PD1 signaling. In some embodiments, the antagonist of PD1 signaling is a polypeptide that targets PD1. In some embodiments, the modulator is an inhibitor comprising a monoclonal antibody or an antigen binding fragment thereof targeting PD1 (CD279). In some embodiments, the antibody or antigen binding fragment thereof comprises one or more of the six CDRs or an antigen binding portion thereof selected from pembrolizumab, nivolumab, cemiplimab, dostarlimab, spartalizumab, camrelizumab, sintilimab, sasanlimab, tiselizumab, or toripalimab.

In some embodiments, the modulator is an inhibitor of the checkpoint protein selected from programmed death-1 ligand 1 (PDL1; CD274; B7-H1), programmed death-1 ligand 2 (PDL2), or both. In some embodiments, the modulator is a polypeptide that targets PDL1, PDL2, or both. In some embodiments, the polypeptide is an antibody or an antigen binding fragment thereof targeting PDL1, PDL2, or both. In some embodiments, the antibody or an antigen binding fragment thereof comprises one or more of the six CDRs or an antigen binding portion thereof selected from any one of durvalumab, avelumab, or atezolizumab.

In some embodiments, the modulator is a polypeptide inhibitor of the checkpoint protein CTLA-4. In some embodiments, the polypeptide is an antibody or an antigen binding fragment thereof targeting CTLA-4. In some embodiments, the antibody or an antigen binding fragment thereof comprises one or more of the six CDRs or an antigen binding portion thereof selected from any one of ipilimumab or tremelimumab.

In some embodiments, the modulator is a polypeptide inhibitor of the checkpoint protein LAG-3. In some embodiments, the polypeptide is an antibody or an antigen binding fragment thereof targeting LAG-3. In some embodiments, the antibody or an antigen binding fragment thereof comprises one or more of the six CDRs or an antigen binding portion thereof selected from any one of relatlimab, fianlimab, Sym022, GSK2831781, TSR-033, iermilimab, favezelimab, tebotelimab, FS118, or pavunalimab.

In some embodiments, the modulator is a polypeptide inhibitor of the checkpoint protein TIGIT. In some embodiments, the polypeptide is an antibody or an antigen binding fragment thereof targeting TIGIT. In some embodiments, the antibody or an antigen binding fragment thereof comprises one or more of the six CDRs or an antigen binding portion thereof selected from any one of tiragolumab, vibostolimab, BMS-986207, ociperlimab, etigilimab, domvanalimab, EOS-448, SEA-TGT, ASP8374, COM902, or IBI939.

In various embodiments, the the second therapeutic agent comprises a modulator that is an agonist of one or more immune stimulatory receptors. In some embodiments, the immune stimulatory receptor is selected from 4-1BB (CD137), Inducible T-cell costimulator (ICOS; CD278), OX-40 (CD134), glucocorticoid-induced TNFR-related protein (GITR; CD357), CD40, Herpesvirus entry mediator (HVEM), CD28, or CD27. In some embodiments, the second therapeutic agent is a polypeptide that comprises CD40L or a CD40-binding fragment thereof. In some embodiments, the second therapeutic agent is a polypeptide that comprises CD80 or CD86; or a CD28-binding fragment thereof.

In various embodiments, the second therapeutic agent comprises a modulator of a cell surface molecule expressed or displayed on a tumor cell or tumor-associated stromal cell. In various embodiments, the cell surface molecule is selected from a growth factor receptor, transforming growth factor-beta receptor (TGFβR), a tumor necrosis factor receptor (TNFR) superfamily receptor, an Ig superfamily receptor, a vascular endothelial growth factor receptor (VEGFR), an epidermal growth factor receptor (EGFR), a platelet-derived growth factor receptor (PDGFR), a tumor cell surface molecule, a cytokine receptor, or a chemokine receptor.

In some embodiments, the second therapeutic agent comprises a modulator of a cell surface molecule expressed or displayed on an immune cell. In some embodiments, the immune cell is a T cell, an NK cell, or a myeloid cell.

In some embodiments, the cell surface molecule is a tumor necrosis factor receptor (TNFR) superfamily receptor, an Ig superfamily receptor, a cytokine receptor, chemokine receptor, T cell co-stimulatory molecule receptor, a T cell co-inhibitory molecule receptor, or a natural killer (NK) cell receptor. In some embodiments, the cell surface molecule is a myeloid cell inhibitory receptor, or a myeloid cell stimulatory receptor.

In various embodiments, the cell surface receptor is SIRPa or CD47. In some embodiments, the second therapeutic agent inhibits the binding of SIRPa to CD47. In some embodiments, the second therapeutic agent is a polypeptide that binds CD47. In some embodiments, the polypeptide is an antibody or antigen binding fragment thereof targeting CD47. In some embodiments, the antibody or an antigen binding fragment thereof comprises one or more of the six CDRs or an antigen binding portion thereof of selected from magrolimab, ZL-1201, TJ011133, STI-6643, SRF231, SHR-1603, IMC-002, IBI188, CC-90002, AO-176, or AK117. In some embodiments, the polypeptide comprises the SIRPa extracellular domain or a CD47-binding fragment thereof. In some embodiments, the polypeptide is selected from evorpacept, TTI-621, or TTI-622. In some embodiments, the second therapeutic agent is polypeptide that binds SIRPa.

In various embodiments, the second therapeutic agent is an antagonist of the signaling of one or more cytokines. In some embodiments, the cytokine is transforming growth factor-beta (TGFb). In some embodiments, the modulator is an inhibitor of TGFb signaling selected from a small molecule kinase inhibitor, polypeptide comprising the TGFbRII ECD or a TGFb-binding fragment thereof, or an antibody or an antigen binding fragment thereof selected from an anti-TGFβ antibody, an anti-TGFβR antibody, an anti-GARP antibody, or an anti-LAP antibody.

In some embodiments, the small molecule kinase inhibitor is a TGFβR small molecule kinase inhibitor comprising galunisertib. In some embodiments, the anti-TGFβ antibody or an antigen binding fragment thereof comprises one or more of the six CDRs or an antigen binding portion thereof fresolimumab. In some embodiments, the polypeptide comprising the TGFbRII ECD or a TGFb-binding fragment thereof is selected from AVID200, bintrafusp alfa (M7824), anti-CTLA4-TGFbRII, SIRPa ECD-TGFbRII, anti-CEA-TGFbRII, anti-PSMA-TGFbRII, anti-IL6R-TGFbRII, anti-PD1-TGFbRII, anti-EGFR-TGFbRII, or anti-HER2-TGFbRII.

In various embodiments, the cytokine is selected from one or more of the following: IL-4, IL-13, IL-10, IL-6, IL-1b, IL-17, IL-22, or VEGF. In some embodiments, the cytokine is IL-4 or IL-13.

In some embodiments, the modulator is a polypeptide that targets IL4 receptor alpha (IL4Ra). In some embodiments, the polypeptide is an antibody or antigen-binding fragment that comprises one or more of the six CDRs or an antigen binding portion thereof of dupilumab.

In some embodiments, the cytokine is IL1b. In some embodiments, the second therapeutic agent comprises anakinra. In some embodiments, the second therapeutic agent comprises one or more of the six CDRs or an antigen binding portion thereof of canakinumab.

In some embodiments, the cytokine is IL10. In some embodiments, the second therapeutic agent comprises an IL 10-binding sequence of the extracellular domain of IL10R, or an antibody or antigen-binding fragment thereof that targets IL10 or IL10R.

In various embodiments, the second therapeutic agent is an agonist of one or more cytokine receptors. In some embodiments, the cytokine receptor is selected from IL12R, IL15R, and IL18R. In some embodiments, the second therapeutic agent is a polypeptide that comprises IL-12.

In various embodiments, the cell surface molecule is a tumor cell surface molecule selected from CA125, CA19-9, CD30, carcinoembryonic antigen-related cell adhesion molecule 1 (CEACAM1), CEACAM5 or cluster of differentiation 66e (CD66e), CEACAM6, DLL3, DLL4, DPEP3, EGFR EGFRVIII, GD2, HER2, HER3, HGF, IGF1R, IL13Ra2, LIV-1, LRRC15, MUC1, PRLR, PSCA, PSMA, PTK7, SEZ6, SLAMF7, TF, cMet, claudin, mesothelin, nectin4, uPAR, GPNMB, CD79b, CD22, NaPi2b, SLTRK6, STEAP1, MUC16, CD37, GCC, AGC-16, 5T4, CD70, TROP2, CD74, CD27L, Fra, CD138, CA6, CD38, SLAMF7, BCMA, CD20, CD19, CD33, or CD30.

In some embodiments, the modulator is an inhibitor comprising a monoclonal antibody or an antigen binding fragment thereof targeting the tumor cell surface molecule. In some embodiments, the antibody or an antigen fragment thereof comprises one or more of the six CDRs or an antigen binding portion thereof selected from any one of labetuzumab, cergutuzumab, cetuximab, necitumumab, panitumumab, depatuxizumab, trastuzumab, pertuzumab, enfortumab, or sacituzumab.

In various embodiments, the immune cell comprises an antigen presenting cell (APC), a myeloid-derived suppressor cell (MDSC), a dendritic cell, a natural killer cell, or a macrophage. In some embodiments, the immune cell comprises, a T_{H}17 cell, a CD4 T cell, a CD8 T cell, a Treg cell, gamma delta T cell, NK cell, innate lymphoid cell (ILC), or gamma delta T17 cell.

In some embodiments, treatment with the combination of the first and second agents reduces or suppresses tumor growth, prevents or reduces severe immune-related adverse events, increases overall survival or progression-free survival, reduces or prevents adverse events or toxicity, or reduces or prevents bone metastases or skeletal-related adverse events, more effectively than treatment with the second agent alone.

In some embodiments, the cancer is selected from prostate cancer, pancreatic cancer, biliary cancer, colon cancer, rectal cancer, liver cancer, kidney cancer, lung cancer, testicular cancer, breast cancer, ovarian cancer, brain cancer, skin cancer, bladder cancer, and head and neck cancers, melanoma, sarcoma, multiple myeloma, leukemia, and/or lymphoma.

In some embodiments, the method further comprises administering to the subject a therapeutic agent comprising an anti-CTLA4-TGFβRII molecule.

In some embodiments, the method further comprises administering one or more anti-cancer therapies. In some embodiments, the one or more anti-cancer therapies comprise a immunotherapeutic agent, chemotherapeutic molecule, antibody, antibody-drug conjugate, small molecule kinase inhibitor, hormonal agent, androgen synthesis inhibitor, androgen receptor antagonist, anti-angiogenic agent, cell therapy, CAR-T cellular therapy, CAR-NK cellular therapy, radionuclide therapy, ionizing radiation, ultraviolet radiation, cryoablation, thermal ablation, a selective estrogen receptor modulator (SERM), a selective estrogen receptor degrader (SERD), or radiofrequency ablation. In some embodiments, the immunotherapeutic agent is selected from immune checkpoint inhibitor, immune stimulatory receptor agonist, immune stimulatory cytokine/cytokine receptor agonist, immune inhibitory cytokine/cytokine receptor antagonist, tumor vaccine, immunomodulatory imide drug, CAR-T cells, CAR-NK cells, or oncolytic virus.

In some embodiments, the subject is a mammal. In some embodiments, the subject is a human.

### BRIEF DESCRIPTION OF THE DRAWINGS

These and other features, aspects, and advantages of the present invention become better understood with regard to the following description, and accompanying drawings, where:

FIGS. 1A-1D show schematics corresponding to multiple exemplary designs of multi-specific polypeptides described herein, in which the IIP comprises an antibody or antibody fragment, and 2P denotes a target binding polypeptide.

FIGS. 2A-2G show schematics corresponding to multiple exemplary designs of multi-specific polypeptides described herein, in which the IIP comprises a ligand-binding fragment of IL-23R ECD. D1, D2 and D3 denote the IL-23R binding domains 1, 2, and 3, respectively. In these figures, dark grey corresponds to the IIP and light grey corresponds to the 2P of the multi-specific polypeptide. 2P denotes a target binding polypeptide.

FIG. 3A depicts a schematic corresponding to an exemplary design of a multi-specific polypeptides with at least one amino acid modification in the PD1 extracellular domain (PD1 ECD A123I or GV-2). FIGS. 3B-3C show that the PD1 ECD A123I or PD1 ECD GV-2 exhibits superior binding affinity for PD1, and FIG. 3D-3E exhibits superior binding affinity for PDL2, when compared to wild-type PD1.

FIGS. 4A-4C demonstrate that IL-23 antibody enhances the antitumor immune response induced by PDL1/PD1 blockade. Combination treatment of anti-IL-23 (anti-IL-23p19) antibody and anti-PD-L1 antibody ((a-IL-23/PDL1 blockade) and exhibited superior survival and smaller tumors compared to mice treated with anti-PDL1 alone (a-PDL1) or anti-IL-23 antibody alone (a-IL-23). FIG. 4A shows combination treatment (a-IL-23/PDL1 blockade) exhibited superior survival. ***p<0.005 by pairwise log-rank test. Y-axis: survival probability. X-axis: number of survival days. FIG. 4B shows tumor volulmn (Y-axis) measurement post tumor inoculation in mice implanted with B16F10 tumor cells. Combination treatment (right panel) reduces tumor volume when compared to the treatment with anti-PD-L1 antibody alone (left panel). FIG. 4C shows reduced pulmonary tumor metastases in mice treated with the combination treatment compared to mice treated with anti-PD-L1 antibody alone. White spots depicts tumor metastases.

FIG. 5 demonstrates an exemplary multi-specific polypeptide as described herein reduces tumor growth and limits toxicity in a humanized mouse model. Treatment with anti-IL-23-PD1ECD reduces tumor volume in NSG mice with hu-PBMCs. *p=0.05 by one-sided t test. Control: vehicle alone; a-IL-23: anti-IL-23 antibody; a-PDL1: anti-PDL1 antibody; a-IL-23-PD1: anti-IL-23-PD1ECD.

FIG. 6 demonstrates an exemplary multi-specific polypeptide as described herein is more effective in inhibiting tumor growth compared to treatment with immune checkpoint inhibitor anti-PD1 antibody (a-PD1) (p=0.03) or combination of checkpoint inhibitors anti-PD1 antibody and anti-CTLA4 antibody (a-PDL1+a-CTLA4). Combination treatment with anti-IL-23-PD1ECD and anti-PD1 antibody and anti-CTLA4 antibody (a-IL-23-PD1+a-CTLA4-TGFbRII) polypeptide inhibits tumor growth and shows statistically synergistic antitumor efficacy (p<0.001) when compared to combined treatment of anti-PD1 antibody and anti-CTLA4 antibody (a-PD1+a-CTLA4).

FIGS. 7A-7B illustrate an exemplary multi-specific polypeptide as described herein (anti-IL-23-PD1ECD), which blocks PDL1 and PDL2 on tumor cells and antigen presenting cells (APCs), and counteracts the Th17/MDSC axis by sequestering IL-23. MDSC: myeloid-derived suppressor cell.

### DETAILED DESCRIPTION

Provided herein are novel polypeptides and methods for the treatment of cancer, autoimmune diseases, and inflammatory disorders, associated with the IL-23 axis.

In the case of cancer, activation of the immune system by an immunotherapeutic agent (e.g., immune checkpoint inhibitor) or other anti-cancer therapy (e.g., a conventional anti-cancer therapy) may lead to the counterproductive activation and expansion of IL-23-dependent immune cells, like Th17, gamma delta T17, and ILC3 cells. These cell types, in the presence of IL-23 signaling that sustains their phenotypes, contribute to suppression of antitumor immunity and/or tumor-promoting inflammation. Furthermore, these cell types contribute to immune-related adverse events and treatment-associated toxicities. As such, the multi-specific polypeptides and methods of treatment as described herein are designed to block IL-23 signaling and simultaneously activate an antitumor immune response. This strategy may enable both superior antitumor efficacy and fewer toxicities (i.e., a broader therapeutic window for immune activation) than approaches solely focused on activating an immune response.

In some cases, the multi-specific polypeptides and combination treatment regimens as described herein comprise one moiety that interferes with IL-23 signaling, and an additional moiety that promotes immune cell activation. In other cases, the additional moiety further helps skew the phenotype of immune cells to an antitumoral state. While breaking tumor-induced immune tolerance by itself may lead to counterproductive activation of IL-23-dependent immune cells, we disclose polypeptides and methods directed towards breaking tumor-induced immune tolerance in the context of IL-23 signaling inhibition.

The counterproductive effects of expanding Th 17 cells in the setting of cancer treatment are not obvious. In fact, a number of studies have suggested infiltration of Th17 cells in the TME are associated with better clinical prognosis; or characterized the role of Th17 cells in cancer as paradoxical. In light of such observations in the field, the multi-specific polypeptides and various combinations described herein, demonstrate the unexpected beneficial effects of IL-23 blockade in conjunction with immune activation (for example, using immune checkpoint inhibitors), both for increasing efficacy and mitigating undesirable immune-related adverse events.

In one aspect, provided herein is a recombinant molecule comprising: (a) an interleukin-23 (IL-23) inhibiting polypeptide (IIP), wherein the IIP comprises IL-23 binding polypeptide or an IL-23R binding polypeptide; and (b) a target binding polypeptide moiety that binds one or more immune checkpoint proteins or immune stimulatory receptors. The IIP can be an IL-23 inhibitor or an IL-23R inhibitor. The targeting binding polypeptide moiety is capable of specifically binding an inhibitor of one or more cell surface molecules or their ligands, such as immune checkpoint protein receptors (e.g., PD-1, PD-L1, PD-L2, CTLA4). Accordingly, the recombinant molecules as described herein are capable of specifically inhibiting IL-23/checkpoint protein (e.g., PD-1) signaling in the tumor microenvironment (TME) and enhancing immune response as well as reducing and/or suppressing tumor growth.

### Definitions

Unless defined otherwise, all technical and scientific terms used herein have the meaning commonly understood by one of ordinary skill in the art to which the invention pertains.

As used herein, the terms “patient” and “subject” are used interchangeably and may be taken to mean any living organism which may be treated with compounds of the present invention. As such, the terms “patient” and “subject” include, but are not limited to, any non-human mammal, primate and human.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. The terms “treat”, “treatment”, and the like regarding a state, disorder or condition may also include (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms.

As used herein “preventing” a disease refers to inhibiting the full development of a disease.

The term “biological sample” refers to any tissue, cell, fluid, or other material derived from an organism (e.g., human subject). In certain embodiments, the biological sample is serum or blood.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (VH) and a heavy chain constant region (CH). The heavy chain constant region typically comprises three domains, abbreviated CH1, CH2, and CH3. Each light chain typically comprises a light chain variable region (VL) and a light chain constant region. The light chain constant region typically comprises one domain, abbreviated CL.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies. One example of an antigen-binding domain is an antigen-binding domain formed by a VH-VL dimer. “Antibody” as used herein encompasses polyclonal and monoclonal antibodies and refers to immunoglobulin molecules of classes IgA (e.g., IgA1 or IgA2), IgD, IgE, IgG (e.g., IgG1, IgG2, IgG3 and IgG4) or IgM, or fragments, or derivatives thereof, including without limitation Fab, F(ab′)2, Fd, single chain antibodies, diabodies, bispecific antibodies, bifunctional antibodies, humanized antibodies, and various derivatives thereof.

The term “antigen binding fragment” refers to a portion of an intact antibody and/or refers to the antigenic determining variable regions of an intact antibody. It is known that the antigen binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

The term “Fc region” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125: S41-52, incorporated by reference in its entirety. The Fc region may be a naturally occurring Fc region, or an Fc region modified as described elsewhere in this disclosure.

The VH and VL regions may be further subdivided into regions of hypervariability (“hypervariable regions (HVRs);” also called “complementarity determining regions” (CDRs)) interspersed with regions that are more conserved. The more conserved regions are called framework regions (FRs). Each VH and VL generally comprises three CDRs and four FRs, arranged in the following order (from N-terminus to C-terminus): FR1—CDR1—FR2—CDR2—FR3—CDR3—FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme); each of which is incorporated by reference in its entirety.

Table 1 provides the positions of CDR1-L (CDR1 of VL), CDR2-L (CDR2 of VL), CDR3-L (CDR3 of VL), CDR1-H (CDR1 of VH), CDR2-H (CDR2 of VH), and CDR3-H (CDR3 of VH), as identified by the Kabat and Chothia schemes. For CDR1-H, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 1
Residues in CDRs according to and Chothia numbering

| CDR | Kabat | Chothia |
|---|---|---|
| CDR1-L | 24-34 | 24-34 |
| CDR2-L | 50-56 | 50-56 |
| CDR3-L | 89-97 | 89-97 |
| CDR1-H (Kabat Numbering) | 31-35B | 26-32 or 34* |
| CDR1-H (Chothia Numbering) | 31-35 | 26-32 |
| CDR2-H | 50-65 | 52-56 |
| CDR3-H | 95-102 | 95-102 |

*The C-terminus of CDR1-H, when numbered using the Kabat numbering convention, varies between 32 and 34, depending on the length of the CDR.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (CH1) of the heavy chain. Fab fragments may be generated, for example, by recombinant methods or by papain digestion of a full-length antibody.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a VH domain and a VL domain in a single polypeptide chain. The VH and VL are generally linked by a peptide linker. See Plückthun A. (1994). In some embodiments, the linker is a (GGGGS) n (SEQ ID NO: 55). In some embodiments, n=1, 2, 3, 4, 5, or 6. See Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell or derived from a non-human source that utilizes a human antibody repertoire or human antibody-encoding sequences (e.g., obtained from human sources or designed de novo). Human antibodies specifically exclude humanized antibodies. In some embodiments, rodents are genetically engineered to replace their rodent antibody sequences with human antibodies.

The term “tumor” refers to all neoplastic cell growth and proliferation, whether malignant or benign, and all pre-cancerous and cancerous cells and tissues. As used herein “cancer” refers to a disease caused by an uncontrolled division of abnormal cells. The terms “cancer,” “neoplastic disease” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer. Non-limiting examples of cancer include prostate cancer, pancreatic cancer, biliary cancer, colon cancer, rectal cancer, liver cancer, kidney cancer, lung cancer, testicular cancer, breast cancer, ovarian cancer, brain cancer, skin cancer, bladder cancer, and head and neck cancers, melanoma, sarcoma, multiple myeloma, leukemia, and/or lymphoma.

The term “immune response” refers to a response of a cell of the immune system (e.g., a B-cell, T-cell, macrophage or polymorphonucleocyte) to a stimulus such as an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines from an actively immunized host to a non-immune host.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed. The term percent “identity” or percent “homology,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. For purposes herein, percent identity and sequence similarity is performed using the BLAST algorithm, which is described in Altschul et al., J. Mol. Biol. 215:403-410 (1990). Software for performing BLAST analyses is publicly available through the National Center for Biotechnology Information (on the world wide web at: ncbi.nlm.nih.gov/).

In some cases, the identity between a reference sequence (query sequence, e.g., a sequence of the disclosure) and a subject sequence, also referred to as a global sequence alignment, can be determined using the FASTDB computer program. In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

The phrase “pharmaceutically acceptable”, as used in connection with compositions described herein, refers to molecular entities and other ingredients of such compositions that are physiologically tolerable and do not typically produce untoward reactions when administered to a subject (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™ or polyethylene glycol (PEG).

A “diluent” as used herein is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The term “pharmaceutical formulation” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

A “stable” formulation is one in which the protein therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N. Y., Pubs. (1991) and Jones, A. Adv. Drug Delivery Rev. 10:29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of aggregation during storage can be used as an indicator of protein stability. Thus, a “stable” formulation may be one wherein less than about 10% and preferably less than about 5% of the protein are present as an aggregate in the formulation. In other embodiments, any increase in aggregate formation during storage of the formulation can be determined.

A “reconstituted” formulation is one which has been prepared by dissolving a lyophilized protein or antibody formulation in a diluent such that the protein is dispersed throughout. The reconstituted formulation is suitable for administration (e.g. subcutaneous administration) to a patient to be treated with the protein of interest and, in certain embodiments, may be one which is suitable for parenteral or intravenous administration.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example. The formulations of the present application can be hypertonic as a result of the addition of salt and/or buffer.

As used herein “immune cells”, “immune effector cells” or “immune responsive cells” include T lymphocytes, B lymphocytes, natural killer (NK) cells, NKT cells, monocytes, macrophages, dendritic cells (DC), antigen presenting cells (APC).

The term “effector T cell” or “T cells” includes T helper (i.e., CD4+) cells and cytotoxic (i.e., CD8+) T cells. CD4+ effector T cells contribute to the development of several immunologic processes, including maturation of B cells into plasma cells and memory B cells, and activation of cytotoxic T cells and macrophages. CD8+ effector T cells destroy virus-infected cells and tumor cells. See Seder and Ahmed, Nature Immunol., 2003, 4:835-842, incorporated by reference in its entirety, for additional information on effector T cells.

The term “regulatory T cell” includes cells that regulate immunological tolerance, for example, by suppressing effector T cells. In some aspects, the regulatory T cell has a CD4+CD25+Foxp3+ phenotype. In some aspects, the regulatory T cell has a CD8+CD25+phenotype. See Nocentini et al., Br. J. Pharmacol., 2012, 165:2089-2099, incorporated by reference in its entirety, for additional information on regulatory T cells.

The term “Th17 cell” includes a subset of CD4+ T cells characterized by signature transcription factor RORgamma and expression of cytokines such as interleukin-17 (IL-17). The term “gamma delta T17 cell” includes a subset of gamma delta T cells similarly characterized by expression of IL-17. The term “ILC3 cells” includes a subset of innate immune cells.

The term “dendritic cell” refers to a professional antigen-presenting cell capable of activating a naïve T cell and stimulating growth and differentiation of a B cell.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminish of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of any of the recombinant molecules, polypeptides, or pharmaceutical compositions provided herein that, when administered to a subject, is effective to treat a disease or disorder.

As used herein, “target binding moiety” or “target binding polypeptide” refers to a molecule that has the ability to localize and bind to a specific molecule or cellular component. The targeting binding moiety or polypeptide can be an antibody, antibody fragment, scFv. Fc-containing polypeptide, fusion antibody, polypeptide, peptide, aptamer, ligand, nucleic acid, or any combination thereof. As a non-limiting example, a targeting moiety or polypeptide is capable of binding to a molecule present in a cell or tissue, a molecule in a diseased cell or tissue (e.g., a cancer cell or tumor), a normal cell or tissue (e.g., an immune cell), a cellular or extracellular molecule that modulates the immune response (e.g., cytokines such as IL-23, or an immune checkpoint protein such as PD-1, CTLA4), a growth factor receptor (e.g., TGFbRII, VEGFR, TNFR, EGFR), growth factor, cytokine receptor, cytokine, or cell surface molecule. As another example, the targeting moiety or polypeptide is a tumor-targeting moiety that is capable of binding a component of a tumor cell or bind in the vicinity of a tumor cell (e.g., tumor vasculature or tumor microenvironment), tumor microenvironment, tumor vasculature, tumor-associated lymphocyte, tumor antigen, tumor-associated antigen, tumor cell surface molecule, tumor antigenic determinant, tumor antigen-containing fusion protein, tumor-associated cell, tumor-associated immune cell, or tumor vaccine. Non limiting examples that a targeting moiety or polypeptide is capable of specifically binding to is a molecule or component including, epidermal growth factor receptor (EGFR, EGFR1, ErbB-1, HER1). ErbB-2 (HER2/neu), ErbB-3/HER3. ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family: AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-α), TGF-α receptor; Transforming growth factor-beta (TGF-β), TGF-β receptor; Interleukin β receptor alpha2 chain (IL 13Ralpha2), Interleukin-6 (IL-6), IL-6 receptor, interleukin-4, IL-4 receptor, cytokine receptors, Class I (hematopoietin family) and Class II (interferon/IL-10 family) receptors, tumor necrosis factor (TNF) family, TNF-α, tumor necrosis factor (TNF) receptor superfamily (TNFRSF), death receptor family, TRAIL-receptor; cancer-testis (CT) antigens, lineage-specific antigens, differentiation antigens, alpha-actinin-4, ARTC1, fibronectin (FN), GPNMB, HLA-A2, MLA-A11, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDX5, PTPRK, IRT2. SNRPD1, SYT-SSX1 or -SSX2 fusion protein, BAGE, BAGE-1-5, GAGE-1-8, MGAT5, LAGE, LAGE-1, CTL-recognixed antigen on melanoma (CAMEL), a member of the melanoma-associated antigen (MAGE) family, mucin 1 (MUC1), MART-1/Melan-A (MLANA), gp100, gp100/Pme117 (SILV), tyrosinase (TYR), TRP-1, HAGE, NA-88, NY-ESO-1, NY-ESO-1/LAGE-2, SAGE, Sp17, SSX-1-4, carcino-embryonic antigen (CEA), Kallikfein 4, mammaglobin-A, OA1, prostate specific antigen (PSA), prostate specific membrane antigen, TRP-2, adipophilin, interferon inducible protein absent in nielanorna 2 (AIM-2), BING-4, CPSF, cyclin D1, epithelial cell adhesion molecule (Ep-CAM), EpbA3, fibroblast growth factor-5 (FGF-5), alpha-feto protein (AFP), M-CSF, MUC1, PBF, FRAME, RAGE-1, RNF43, RU2AS, SOX10, STEAP1, XAGE, ADAM2, PAGE-5, LIPI, CTAGE-1, CSAGE, MMAI, CAGE, BORIS, HOM-TES-85, AF15q14, HCA66I, LDHC, MORC, SGY-1, SPO11, TPX1, NY-SAR-35, FTHLI7, TDRD1, TEX 15, FATE, TPTE, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, GM2, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2). A composition of the invention can further include the foregoing as a peptide/polypeptide and/or encoding the same.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with a multi-specific polypeptide provided herein. In some aspects, the disease or condition is a cancer. In some aspects, the disease or condition is an immune disorder.

The term “immune stimulatory receptor” refers to a polypeptide expressed on the cell surface of an immune cell that results in activation, maturation, proliferation, or stimulation of said cell. In some aspects, an immune stimulatory receptor may signal via one or more intracellular immunoreceptor tyrosine-based activation motifs (ITAMs) or immunoreceptor tyrosine-based switch motifs (ITSMs). A subset of immune stimulatory receptors expressed on T cells may be referred to as “T cell co-stimulatory receptors.” In some aspects, ligation of a T cell co-stimulatory receptor by its cognate ligand results in intracellular signaling that activates the T cell. In some aspects, this “Signal 2” acts in concert with “Signal 1” resulting from TCR ligation to fully activate the T cell. Non-limiting examples of T cell co-stimulatory receptors include 4-1BB (CD137), Inducible T-cell costimulator (ICOS; CD278), OX-40 (CD134), glucocorticoid-induced TNFR-related protein (GITR; CD357), Herpesvirus entry mediator (HVEM), CD28, or CD27. A subset of immune stimulatory receptors expressed on innate immune cells may be referred to as “innate immune stimulatory receptors”. Non-limiting examples of innate immune stimulatory receptors expressed on NK cells include TRAIL, CD16, NKp30, NKp44, NKp46, NKp80, NKG2C, NKG2D, 2B4 (CD244), DNAM-1 (CD226), CD137, OX40, and CD27. Non-limiting examples of innate immune stimulatory receptors expressed on myeloid cells include DAP12 and Fc receptor gamma, and receptors that are coupled to ITAM-containing adaptors like DAP12 and Fc receptor gamma, such as TREM-2.

The term “immune checkpoint protein” refers to a polypeptide that attenuates the activation of an immune cell. In some aspects, immune checkpoint proteins include receptors that transduce inhibitory signals in an immune cell (e.g., PD-1) and ligands that activate such receptors (e.g., PD-L1, PD-L2). In other aspects, immune checkpoint proteins include receptors that sequester ligands of immune stimulatory receptors (e.g., CTLA-4 sequesters immune stimulatory ligands CD80, CD86 thereby preventing their interaction with immune stimulatory receptor CD28). In some aspects, immune checkpoint proteins may signal via one or more intracellular immunoreceptor tyrosine-based inhibitory motifs (ITIMs) or immunoreceptor tyrosine-based switch motifs (ITSMs). A subset of immune checkpoint receptors expressed on T cells may be referred to as “T cell co-inhibitory receptors”, and their cognate ligands as “T cell co-inhibitory ligands”. A subset of immune checkpoint receptors expressed on innate immune cells may be referred to as “innate inhibitory receptors” and their cognate ligands as “innate inhibitory ligands.”

The term “immunogenic chemotherapeutic agent” refers to a chemotherapeutic agent that leads to immunogenic cell death of cancer cells. Immunogenic cell death refers to any mechanism wherein cell death is able to drive an antigen-specific immune response. Numerous anti-cancer therapies including chemotherapy, radiation, and targeted therapies are able to induce immunogenic cell death. In some aspects, immunogenic chemotherapeutic agents are genotoxic. Non-limiting example of classes of chemotherapeutic agents that can cause immunogenic cell death include alkylating agents (e.g., cyclophosphamide, ifosfamide), topoisomerase inhibitors (e.g., doxorubicin), platinum derivatives (e.g., cisplatin, carboplatin, oxaliplatin, nedaplatin), taxanes (e.g., paclitaxel, docetaxel), or anthracyclines (e.g., doxorubicin). In some aspects, an immunogenic chemotherapeutic agent or derivative thereof may be conjugated to an antibody or other polypeptide for delivery as an antibody-drug conjugate.

The term “immune inhibitory enzyme” refers to an enzyme whose metabolic activity has an immunosuppressive effect. In some embodiments, the immune inhibitory enzyme is an ectonucleotidase (e.g., CD39, CD73) or indoleamine 2,3-dioxygenase. Non-limiting examples of suitable immune inhibitory enzymes include quiescin sulfhydryl oxidase 1 (QSOX1), carbonic anhydrase 12 (CA12), and Carbonic anhydrase IX (CAIX).

As used herein, a “modulator” of a particular target refers to an agent that, without limitation, in certain embodiments may bind said target and inhibit the activity of said target (i.e., act as an antagonist), or in alternative embodiments, a modulator may promote the activity of said target (i.e., act as an agonist). A modulator may inhibit or promote the activity of a given target directly or indirectly (for example, by binding its cognate binding partner, a molecule upstream in its signaling, or a molecule downstream in its signaling).

The term “tumor stromal cell” refers to non-malignant cells in the tumor microenvironment. In one aspect, tumor stromal cells are components of the structural or connective tissue in a tumor. In another aspect, tumor stromal cells form or participate in the formation of blood vessels. Non-limiting examples of tumor stromal cells include fibroblasts, cancer-associated fibroblasts (CAFs), vascular endothelial cells, pericytes, adippocytes, mesenchymal stromal cells, and myofibroblasts.

The term “immune related adverse events” (irAEs) refers to any undesirable side effects caused by immune activation. irAEs may include gastrointestinal, endocrine, cardiac, pulmonary, hepatic, rheumatalogical, renal, neurological or dermatologic/cutaneous toxicities. irAEs may be caused by or associated with treatment with immune checkpoint inhibitors, or other anti-cancer therapies that cause or are associated with activation of immune cells. irAEs are further defined and reviewed in Martins et al., “Adverse effects of immune-checkpoint inhibitors: epidemiology, management and surveillance.” Nat Rev Clin Oncol 2019; 16:563, which is incorporated herein in its entirety.

The symbol “+” in the sequence listing table means fusion of the polypeptide sequences indicated. For example, “A+B” means fusion of A to B, in the order indicated (i.e., N terminus -A-B-C terminus).

Multi-Specific Polypeptides that Block IL-23/IL-23R

IL-23 is one component of the tumor microenvironment (TME) that is involved in development, progression, and metastasis of malignant cells. IL-23 may manipulate host immune responses, modulate the cells in TME, and directly affect a variety of premalignant and malignant tumors.

Treatment of cancer with agents that promote immune cell activation can result in the activation of T cell subsets that inhibit antitumor immunity and/or contribute to tumorigenic inflammatory signaling. For example, Th17 cells are a subset of CD4 T cells characterized by expression of signature transcription factor RORg and expression of inflammatory cytokines like IL-17, IL-21, IL-22, IL-1, and TNFα. Without being bound to any theories, Th 17 cells can directly and indirectly promote tumor progression by activating fibroblasts leading to fibrosis, producing cytokines that contribute to epithelial cell survival/proliferation, and promoting angiogenesis through endothelial cell activation and ECM remodeling. Th17 cells can recruit and activate myeloid cells that exert independent immunosuppressive programs that inhibit antitumor immunity, such as myeloid-derived suppressor cells (MDSCs). Therefore, treatment of cancer with agents that promote immune cell activation may result in the counterproductive activation/expansion of inflammatory T cells like Th17 cells, gamma delta T17 cells, and ILC3 cells which inhibit antitumor immunity and/or contribute to tumorigenic inflammatory signaling. Furthermore, IL17+ T cells such as Th17 cells and gamma delta T17 cells are associated with induction of immune related adverse events (irAEs) and toxicity in response to immunotherapy. This may limit the therapeutic window of immunotherapeutic agents, or more generally, any immunogenic anti-cancer agent. IL-23 is a STAT3-activating cytokine that plays a pivotal role in the differentiation and maintenance of these inflammatory cell phenotypes, such as Th17 cells, gamma delta T17 cells, and ILC3 cells. In addition, IL-23 and resultant STAT3 signaling employs multiple mechanisms to inhibit IL-12 signaling and resultant STAT4 signaling, limiting the differentiation and maintenance of the antitumoral Th1 T cell phenotype. Therefore, IL-23 blockade may increase the efficacy and safety of therapeutic strategies that aim to enhance immune cell activation, proliferation, and/or function. Non-limiting examples of such therapeutic strategies include antagonism of immune checkpoint proteins (including T cell co-inhibitory receptors and innate inhibitory receptors), agonism of immune stimulatory receptors (including T cell co-stimulatory receptors and innate stimulatory receptors), antagonism of particular cytokines/cytokine receptors, agonism of particular cytokine receptors, immunogenic chemotherapy, antagonism of immune inhibitory enzymes, and administration of cellular therapy comprising CAR-T, CAR-NK, or hematopoietic stem cells. Such therapeutic strategies for the treatment of cancer are sometimes described as “breaking tolerance”, or attempting to do so. As such, in some aspects, the molecules and methods of this invention offer strategies to break tolerance while simultaneously counteracting IL-23, a principal determinant of skewing of immune cell phenotypes into a tumor-promoting state. This can enable breaking tolerance while ensuring the phenotypes of immune cells in the tumor microenvironment do not polarize towards such a counterproductive state, and as such, enhance both the safety and efficacy of therapies that aim to break tolerance.

Provided herein are multi-specific polypeptides that are capable of specifically blocking IL-23/IL-23 receptor (IL-23/IL-23R) signaling. The multi-specific polypeptide comprises at least two moieties: (a) an IL-23 inhibiting polypeptide (IIP), and (b) a secondary polypeptide (2P) such as a target binding polypeptide. In preferred embodiments, the multi-specific polypeptides disclosed herein effectively inhibits tumor growth and/or reduces tumor volume compared to treatment with immune checkpoint inhibitors alone. In another preferred embodiments, the multi-specific polypeptides disclosed herein prevents or reduces an immune disorder such as graft-versus-host disease.

In some embodiments, the IL-23 inhibiting polypeptide inhibits the IL-23/IL-23R signaling by blocking or interfering with the interaction of the IL-23 ligand and IL-23 receptor. In some embodiments, the IL-23 inhibiting polypeptide is capable of specifically binding the IL-23 ligand and depletes IL-23 binding to the IL-23 receptor presented on a cell surface (e.g., T cell, natural killer cell, natural killer T cell, dentritic cell, macrphaage, tumor cell). In some embodiments, the IL-23 inhibiting polypeptide is capable of specifically binding the IL-23 receptor presented on a cell surface. In some embodiments, the IL-23 inhibiting polypeptide is an anti-IL-23 antibody or an antigen-binding fragment thereof. In some embodiments, the IL-23 inhibiting polypeptide is an anti-IL-23R antibody or an antigen-binding fragment thereof. In some embodiments, the IL-23 inhibiting polypeptide comprises the extracellular domain of the IL-23 receptor (IL-23 ECD) and is capable of binding the IL-23 ligand, thereby preventing IL-23 from binding the endogenous IL-23R and inhibiting IL-23 signaling.

In some embodiments, the secondary polypeptide (2P) is a target binding polypeptide that binds one or more immune checkpoint proteins expressed or presented on the cell surface of an immune cell (e.g., antigen presenting cell, CD4+ T cell, Th 17 cell) or a tumor cell, thereby blocking or interfering the interaction of the immune checkpoint proteins. In some embodiments, the target binding polypeptide binds an immune checkpoint protein, a receptor or a ligand binding fragment thereof, or a ligand or a receptor binding fragment thereof selected from programmed death-1 (PD1; CD279), programmed death ligand 1 (PDL1; CD274; B7-H1), programmed death ligand 2 (PDL2), cytotoxic T-lymphocyte antigen-4 (CTLA4; CD152), B and T lymphocyte attenuator (BTLA), V-domain immunoglobulin suppressor of T cell activation (VISTA), T cell immunoglobulin and ITIM domain (TIGIT), lymphocyte-activation gene 3 (LAG-3; CD223), T-cell immunoglobulin and mucin domain 3 (Tim-3; HAVCR2), carcinoembryonic antigen-related cell-adhesion molecule 1 (CEACAM1), CD47, signal regulatory protein alpha (SIRPa), Major Histocompatibility Complex, Class I, G (HLA-G), Ig-like transcript 2 (ILT2; LILRB1), Ig-like transcript 4 (ILT4, LILRB2) or combinations thereof. In some embodiments, the target binding polypeptide binds PD1 (CD279). In some embodiments, the target binding polypeptide is an anti-PD1 antibody or an antigen-binding fragment thereof. In some embodiments, the target binding polypeptide binds PDL1 (CD274; B7-H1). In some embodiments, the target binding polypeptide is an anti-PDL1 antibody or an antigen-binding fragment thereof. In some embodiments, the target binding polypeptide binds PDL2. In some embodiments, the target binding polypeptide is an anti-PDL2 antibody or an antigen-binding fragment thereof. In some embodiments, the target binding polypeptide comprises the extracellular domain of PD1 (PD1 ECD) capable of binding a ligand or a receptor binding fragment of, e.g., PDL1, PDL2. In some embodiments, the PD1 ECD has one or more mutations relative to the wild type human PD1 ECD (SEQ ID NO: 56) to increase its binding affinity to PDL1 and/or PDL2. The PD1 ECD may comprise a mutation of residue A132 (residue numbering as defined by full human PD1 sequence, as in UniProt Q15116). The PD1 ECD may comprise a conservative substitution of residue A132. In a preferred embodiment, the PD1 ECD comprises the mutation A132I (SEQ ID NO: 57). In some embodiments, the PD1 ECD variant binds PDL1 with an affinity greater than 100 nM, 10 nM, 1 nM, or 0.1 nM. In some embodiments, the PD1 ECD variant binds PDL2 with affinity greater than 100 nM, 10 nM, 1 nM, or 0.1 nM.

Tumor cells and myeloid-derived suppressor cells (MDSCs) may express PDL1 and/or PDL2. Tumor cells and MDSCs also may express IL-23. As such, a multi-specific polypeptide that binds PDL1 and/or PDL2; and IL-23, may localize blockade of IL-23 to the cell surface of PDL1+ and/or PDL2+ cells that also express IL-23 (e.g., a cell that presents or expresses IL-23, and PDL1 or PDL2, or both).

### IL-23-Inhibiting Polypeptide

Accordingly, in some embodiments, the IL-23-inhibiting polypeptide (IIP) inhibits IL-23/IL-23R signaling in one of the following ways: (a) inhibiting the interaction of IL-23 and IL-23R by binding IL-23 (IIP is an “IL-23 binder”), or (b) inhibiting the interaction of IL-23 and IL-23R by binding IL-23R (IIP is an “IL-23R binder”).

### IL-23 Binder

In some embodiments, the IL-23-inhibiting polypeptide binds IL-23. In some embodiments, the IL-23-inhibiting polypeptide is an anti-IL-23 antibody. In some embodiments, the anti-IL-23 antibody is humanized monoclonal antibody or an antigen binding fragment thereof capable of specifically binding an IL-23 subunit (e.g., IL-23p19, IL-23p40). In some embodiments, the IL-23 antibody or an antigen binding fragment thereof is capable of specifically binding the IL-23p19 subunit. As a non-limiting example, the IL-23 antibody or an antigen binding fragment thereof comprises one or more of the six complementarity-determining regions (CDRs) selected from any one of risankizumab (VH: SEQ ID NO: 79; VL: SEQ ID NO: 80), guselkumab (VH: SEQ ID NO: 81; VL: SEQ ID NO: 82), tildrakizumab (VH: SEQ ID NO: 83; VL: SEQ ID NO: 84), brazikumab (VH: SEQ ID NO: 85; VL: SEQ ID NO: 86), and mirikizumab (VH: SEQ ID NO: 87; VL: SEQ ID NO: 88).

### IL-23R Binder

In some embodiments, the IL-23-inhibiting polypeptide binds IL-23R. In some embodiments, the IL-23-inhibiting polypeptide is an anti-IL-23R antibody. In some embodiments, the anti-IL-23R antibody is humanized monoclonal antibody or an antigen binding fragment thereof capable of specifically binding an IL-23 ligand or a receptor binding fragment thereof. As a non-limiting example, the IL-23R antibody or an antigen binding fragment thereof comprises one or more of the six CDRs of AS2762900-00. In some embodiments, the IL-23R antibody or antigen-binding fragment thereof is selected from an antibody disclosed in U.S. Pat. No. 9,371,391 which is incorporated herein in its entirety.

### IL-23R ECD

In some embodiments, the IL-23-inhibiting polypeptide comprises the IL-23 receptor, a ligand binding domain or fragment thereof, or an extracellular domain (IL-23R-ECD) thereof capable of specifically binding endogenous IL-23 ligands. For example, the IL-23R-ECD may be the D1 subunit of IL-23R, the D1 and D2 subunits of IL-23R, or the D1, D2, and D3 subunits of IL-23R. The sequence of human IL-23R and its extracellular domain have been reported. As used herein, the ECD sequence has an amino acid sequence of SEQ ID NO: 1 (UniProt accession Q5VWK5).

The IL-23 ligand is a heterodimeric cytokine comprising the p19 and p40 subunits. The IL-12 ligand is a heterodimeric cytokine comprising the p35 and p40 subunits. In one aspect, IL-23R-ECD of the fusion proteins of the invention displays higher affinity for IL-23 than IL-12. In one embodiment, IL-23R-ECD preferentially binds the p19 subunit compared to the p35 subunit. In some embodiments, the IL-23R-ECD comprises residues that interact with both the p19 and p40 subunits of IL-23. In other embodiments, the IL-23R-ECD comprises residues that only interact with the p19 subunit.

In some embodiments, the IL-23R-ECD domain includes the GITNIN hexapeptide that is upstream of D1. In other embodiments, the IL-23R-ECD domain begins with the amino acid sequence at the start of D1 (CSGHI).

In some embodiments, IL-23R-ECD as used herein may be modified in one or more of the following ways, as reference to the native human IL-23-R extracellular domain (e.g., wild type IL-23R-ECD) sequence (SEQ ID NO: 1). The IL-23R-ECD may have one or more substitutions or deletions of residues that are not necessary for ligand binding, one or more substitutions of residues to remove N-linked glycosylation sites, one or more substitutions, additions, or deletions of residues to increase affinity to IL-23, one or more substitutions, additions, or deletions of residues to improve the expression of the fusion protein, one or more substitutions, additions, or deletions of residues to allow for site-specific conjugation of drug conjugates, one or more substitutions, additions, or deletions of residues to decrease the specificity of the ligand trap to IL-12 while maintaining or increasing its specificity to IL-23, a fusion of one or more non-continuous domains of IL-23R-ECD, or a fusion of domains from different isoforms of IL-23R-ECD.

In some embodiments, the IL-23R-ECD has an amino acid sequence having at least 40%, 50%, 60%, 70%, 80%, 90%, 95%, 98%, 99% or more sequence identity to a ligand-binding sequence of wild type human IL-23R-ECD (SEQ ID NO: 1).

### Secondary Polypeptide (2P)

In some embodiments, the secondary polypeptide is a target binding polypeptide and that serves one or more of the following functions such as (a) localization of the multi-specific polypeptide to a specific tissue, cell type, or tumor cell; (b) antagonism of inhibitory immune checkpoint signaling; (c) agonism of immune stimulatory signaling; (d) antagonism of another cytokine or cytokine receptor, (e) agonism of a cytokine receptor, and/or (f) antagonism of a chemokine or chemokine receptor.

In various embodiments, the secondary polypeptide (2P) is a target binding polypeptide that is an antigen-binding domain of an immunoglobulin, antibody, bispecific or multispecific antibody, nanobody, antibody fragment, single chain variable fragment (scFv), bivalent or multivalent scFv, Affimer, a ligand-binding sequence from the extracellular domain (ECD) of a receptor, a receptor-binding sequence from the ECD of a ligand, or Fc-containing polypeptide.

In some embodiments, 2P is a target binding polypeptide that is an antibody or an antigen binding fragment thereof such as a fragment crystallizable (Fc) region, a fragment antigen binding (Fab) region, a single chain variable fragment (scFv), a light chain or a functional portion thereof, a variable region of the light chain (VL), a constant region of the light chain (CL), a heavy chain or a functional portion thereof, a variable region of the heavy chain (VH), a constant region of the heavy chain (CH), at least one complementarity-determining region (CDR) or an antigen-binding portion thereof, or combinations thereof.

In some embodiments, 2P is a target binding polypeptide having a ligand-binding sequence of the extracellular domain of a receptor. In some embodiments, 2P is a target binding polypeptide having a receptor-binding sequence of the extracellular domain of a ligand. In some embodiments, the ECD has one or more of the following modifications as reference to the wild type ECD. In various embodiments, the ECD has one or more substitutions, additions, or deletions of residues to improve the expression of the fusion protein, one or more substitutions or deletions of residues that are not necessary for ligand binding, one or more substitutions of residues to remove N-linked glycosylation sites, or one or more substitutions, additions, or deletions of residues to increase affinity to the native binding partner of the ECD.

### Examples of 2P Moiety for Treating Cancer or Immune Disorders

In various embodiments, the secondary polypeptide (2P) is a target binding polypeptide capable of specifically binding one or more cytokines or cytokine receptors, or one or more cell surface molecules. In some embodiments, 2P is a target binding polypeptide that allows exchange of the fusion protein through the blood-brain barrier. In some embodiments, 2P is a target binding polypeptide comprising, a Fc domain, a CDR, or an antigen binding fragment of an immunoglobulin.

In some embodiments, the 2P binds a cytokine or cytokine receptor that promotes the differentiation, maturation, or function of T_{H}17 cells. In some embodiments, the 2P binds IL-17 or IL-17R. In some embodiments, the 2P is an antibody or an antigen binding fragment thereof that binds and disables IL-17 or IL-17R. In some embodiments, the 2P antibody or antigen binding fragment thereof comprises one or more of the six CDRs or an antigen binding portion thereof selected from any one of afasevikumab, bimekizumab, ixekizumab, netakimab, perakizumab, secukinumab, vunakizumab, or brodalumab. In some embodiments, the 2P is a ligand-binding sequence of the extracellular domain of IL-17R or a fragment thereof. In some embodiments, the 2P binds IL-1a, IL-1b, or IL-1R. In some embodiments, the 2P is an antibody or an antigen binding fragment thereof that binds and disables IL-1a, IL-1b, and/or IL-1R. In some embodiments, the 2P is a ligand-binding sequence of the extracellular domain of IL-1R, IL-1 receptor antagonist (IL-IRA) (SEQ ID NO: 77) or a fragment thereof. In some embodiments, the 2P comprises the amino acid sequence of anakinra. In some embodiments, the 2P binds IL-6 or IL-6R. In some embodiments, the 2P is an antibody or an antigen binding fragment thereof that binds and disables IL-6 or IL-6R. In some embodiments, the 2P antibody or antigen binding fragment thereof comprises one or more of the six CDRs or an antigen binding portion thereof selected from any one of clazakizumab, olokizumab, siltuximab, sirukumab, ziltivekimab, levilimab, sapelizumab, sarilumab, satralizumab, or tocilizumab. In some embodiments, the 2P prevents the interaction of RANK with RANKL. In some embodiments, the 2P is a RANKL-binding sequence of the extracellular domain of RANK (SEQ ID NO: 76).

In some embodiments, the 2P binds a TNFR superfamily receptor or a ligand that binds a TNFR superfamily receptor. In other embodiments, the 2P binds a type I cytokine receptor or a cytokine that binds a type I cytokine receptor. In other embodiments, the 2P binds a type II cytokine receptor or a cytokine that binds a type II cytokine receptor. In other embodiments, the 2P binds an Ig superfamily receptor or a cytokine that binds an Ig superfamily receptor. In other embodiments, the 2P binds a chemokine receptor or a chemokine that binds a chemokine receptor.

In some embodiments, the 2P binds a cell surface molecule of a cell responsible for producing IL-23, thereby sequestering IL-23 as it is expressed. In other embodiments, the 2P binds a cell surface molecule of a cell that expresses IL-23R and normally is responsive to IL-23, thereby sequestering IL-23 on a cell that would otherwise initiate IL-23R signaling.

In some embodiments, the 2P binds a T cell surface molecule. The fusion protein may be designed to counteract inflammation mediated by T_{H}17 cells. As such, in some embodiments, the 2P binds a T cell surface molecule preferentially expressed by T_{H}17 cells. In other embodiments, the 2P binds a T cell surface molecule expressed by CD4 T cells.

In some embodiments, the 2P binds to the transferrin receptor (TfR). Without being bound to any theories, binding of 2P to TfR allows exchange of the fusion protein through the blood-brain barrier. In some embodiments, the 2P is an antibody or an antigen binding fragment thereof that binds TfR. In other embodiments, the 2P is an antibody or an antigen binding fragment thereof with an engineered Fc region mutated to bind TfR. In other embodiments, the 2P comprises a synthetic peptide sequence engineered to bind TfR. In some embodiments, the engineered Fc region comprises SEQ ID NO: 116. In some embodiments, the engineered Fc region comprises one or more mutations disclosed in Kariolis et al, “Brain delivery of therapeutic proteins using an Fc fragment blood-brain barrier transport vehicle in mice and monkeys” Sci Trans Med 2020, 12:545, which is incorporated herein by reference in its entirety.

In some embodiments, the 2P comprises the Fc domain of an immunoglobulin. In some embodiments, the Fc domain is a wild type IgG. In some embodiments, the Fc domain possess one or more mutations designed to enhance or abrogate its binding to various Fc receptors. In some embodiments, the Fc domain is an IgG1 Fc comprising the L234A and/or L235A (“LALA”) mutations. In some embodiments, the Fc domain is a IgG4 Fc comprising the S228P mutation.

### Examples of 2P Moiety for Treating Cancer

In various embodiments, the 2P has target binding polypeptide capable of binding one or more target molecules for treating cancer. In some embodiments, the 2P binds a tumor cell surface molecule. Without being bound to any theories, the 2P may serve to localize the fusion protein to the tumor cell surface, ‘decorating’ it with an IL-23 binder to sequester any IL-23 in the tumor cell microenvironment. Binding of the 2P to its target may additionally serve to neutralize a receptor/ligand interaction that aggravates immune tolerance or tumor promoting inflammation; or to neutralize a growth factor, growth factor receptor, or other molecule that promotes tumor cell survival, growth, or metastases. In various embodiments, the tumor cell surface molecule is a T cell co-inhibitory ligand, a tumor growth factor receptor, a cytokine receptor, a chemokine receptor, or a tumor antigen. In some embodiments, the 2P is a antibody or an antigen binding fragment thereof that binds a specific tumor cell surface molecule. As a non-limiting example, the 2P binds a tumor cell surface molecule selected from the following list including CA125, CA19-9, CD30, carcinoembryonic antigen-related cell adhesion molecule 5 (CEACAM5) or CD66e (e.g., labetuzumab, cergutuzumab), CEACAM1, CEACAM6, DLL3, DLL4, DPEP3, EGFR (e.g., cetuximab, necitumumab, panitumumab), EGFRvIII (e.g. depatuxizumab), GD2, HER2 (e.g., trastuzumab, pertuzumab), HER3, HGF, IGF1R, IL13Ra2, LIV-1, LRRC15, MUC1, PRLR, PSCA, PSMA, PTK7, SEZ6, SLAMF7, TF, cMet, claudin, mesothelin, nectin4 (e.g., enfortumab), uPAR, GPNMB, CD79b, CD22, NaPi2b, SLTRK6, STEAP1, MUC16, CD37, GCC, AGC-16, 5T4, CD70, TROP2 (e.g., sacituzumab), CD74, CD27L, Fra, CD138, and CA6. In various embodiments, the 2P binds a cell surface molecule of a tumor stromal cell.

In some embodiments, the 2P binds CEA (CEACAM5). In some embodiments, the 2P is labetuzumab. In another embodiment, the 2P is cergutuzumab or CH1A1A-2F1. In one embodiment, the 2P binds membrane-bound CEA preferentially over soluble CEA. In some embodiments, this is achieved by binding a CEA epitope near the GPI-anchoring site at the C-terminus of the CEA extracellular domain. In some embodiments, this is achieved by binding a CEA epitope overlapping with the B3 domain of CEA.

In some embodiments, the 2P binds an antigen overexpressed by a hematologic malignancy. In some embodiments, the 2P binds an antigen overexpressed by multiple myeloma. In some embodiments, the 2P binds CD38, SLAMF7, or BCMA. In some embodiments, the 2P is an antibody selected from the following list: MEDI2228; CC-99712; belantamab; Gemtuzumab (anti-CD33 mAb). In some embodiments, the antibody binds CD20. In some embodiments, the 2P binds rituximab (chimeric murine/human anti-CD20 mAb); Obinutuzumab (anti-CD20 mAb); Ofatumumab (anti-CD20 mAb). In some embodiments, the 2P binds CD19. In some embodiments, the antibody binds CD30, or CD22. In some embodiments, the 2P binds an antigen overexpressed by leukemia. In some embodiments, the 2P binds CD33.

In some embodiments, the 2P is an antagonist of an immune checkpoint protein. In some embodiments, the 2P is an antagonist of an innate immune checkpoint protein. In some embodiments, the 2P binds a T cell co-inhibitory molecule as an antagonist. In some embodiments, the 2P has a ligand-binding sequence of the extracellular domain of a T cell co-inhibitory receptor. Such a 2P has the effect of sequestering the T cell co-inhibitory ligand, diminishing ligand-induced signaling of the native T cell co-inhibitory receptor expressed on the T cell surface.

In some embodiments, the 2P has a ligand-binding sequence of the PD1 ECD. The PD1 ECD may comprise one or more mutations relative to the wild type human PD1 ECD to increase its binding affinity to PDL1 and/or PDL2. In some embodiments, the PD1 ECD has a mutation of residue A132 (residue numbering as defined by full human PD1 sequence, as in UniProt Q15116). The A132 residue may be mutated (e.g., substitution, deletion, insertion, or inversion) to I (A132I), V (A132V), or L (A132L). In a preferred embodiment, the PD1 ECD has a mutation at A132I. In some embodiments, the PD1 ECD has multiple amino acid mutations to increase its binding affinity to PDL1 and/or PDL2, including but are not limited to, S87G, P89L, N116S, G124S, S127V, A140V, A125I, A125V, L122V, K78T, N74G, M70E, Y68H, N66V, N66I, L65I, L65V, V64H or additional amino acid mutations as described in Miao et al., “Neutralization of PD-L2 is Essential for Overcoming Immune Checkpoint Blockade Resistance in Ovarian Cancer.” Clin Cancer Res. 2021. 27 (15): 4435-4448, Maute et al., “Engineering high-affinity PD-1 variants for optimized immunotherapy and immuno-PET imaging.” Proceedings of the National Academy of Sciences. 2015. 112 (47), E6506-E6514, each of which is incorporated herein in its entirety.

In other embodiments, the 2P comprises a ligand-binding sequence of TIM3 ECD (SEQ ID NO: 63). In other embodiments, the 2P comprises a CTLA4-binding sequence of CD80 ECD (SEQ ID NO: 66) or CD86 ECD (SEQ ID NO: 67).

In some embodiments, the 2P binds an immune stimulatory receptor as an agonist. In some embodiments, the 2P binds a T cell co-stimulatory molecule as an agonist. In some embodiments, the immune stimulatory receptor may be selected from 4-1BB (CD137), Inducible T-cell costimulator (ICOS; CD278), OX-40 (CD134), glucocorticoid-induced TNFR-related protein (GITR; CD357), CD40, Herpesvirus entry mediator (HVEM), CD28, or CD27. In some embodiments, the 2P comprises a receptor-binding sequence of a T cell co-stimulatory ligand, or a receptor binding fragment thereof. In some embodiments, the 2P comprises a CD40-binding sequence of CD40L (SEQ ID NO: 74). In some embodiments, the 2P comprises multiple CD40L moieties such that they assemble into a trimeric or hexameric configuration. In some embodiments, the 2P comprises a receptor-binding sequence of ICOS-L (SEQ ID NO: 73), 4-1BBL (SEQ ID NO: 70), OX40L (SEQ ID NO: 71) or GITRL (SEQ ID NO: 72). In some embodiments, the 2P comprises an HVEM-binding sequence of BTLA ECD (SEQ ID NO: 62) or LIGHT ECD (SEQ ID NO: 65). In some embodiments, the 2P comprises a CD28-binding sequence of CD80 ECD or CD86 ECD.

In some embodiments, the 2P binds an innate immune stimulatory receptor as an agonist. In some embodiments, the 2P binds an innate immune stimulatory receptor expressed on NK cells as an agonist. In some embodiments, the 2P binds NKG2D as an agonist. In some embodiments, the 2P comprises a NKG2D-binding sequence of NKG2D ligand (NKG2DL).

In some embodiments, the 2P binds a growth factor or growth factor receptor. In some embodiments, the 2P inhibits TGFb signaling. In some embodiments, the 2P binds TGFb and prevents it from binding TGFbRII. In some embodiments, the 2P comprises a ligand-binding sequence of the TGFbRII ECD (SEQ ID NO: 58). In some embodiments, the 2P is an anti-TGFb antibody or an antigen binding fragment thereof (e.g., fresolimumab, SRK-181, SAR439459, NIS793). In other embodiments, the 2P binds TGFbRII. In some embodiments, the 2P is an anti-TGFbRII antibody or an antigen binding fragment thereof. In some embodiments, the multi-specific polypeptide is anti-IL-23-TGFbRII, comprising amino acid sequences SEQ ID NO: 90 and SEQ ID NO: 54.

In some embodiments, the 2P inhibits the interaction of VEGF and VEGFR. In some embodiments, the 2P binds VEGF. In some embodiments, the 2P is a ligand-binding sequence of the extracellular domain of VEGFR1 (SEQ ID NO: 59) or VEGFR2 (SEQ ID NO: 60); or a chimeric ECD comprising domains from VEGFR1 and VEGFR2. In some embodiments, the chimeric ECD comprises VEGFR1 domain 2 and VEGFR2 domain 3 (SEQ ID NO: 61). In some embodiments, the 2P is aflibercept. In some embodiments, the 2P is an antibody or an antigen binding fragment thereof that binds VEGF (e.g., bevacizumab). In some embodiments, the 2P binds VEGFR. In some embodiments, the 2P is an antibody or an antigen binding fragment thereof binds VEGF (e.g., ramucirumab). In some embodiments, the multi-specific polypeptide is anti-IL-23-VEGFR, comprising amino acid sequences SEQ ID NOs. 91 and 54.

In some embodiments, the 2P binds and neutralizes a molecule expressed on the cell surface of a dendritic cell or macrophage. In some embodiments, the 2P binds and neutralizes a molecule expressed on the cell surface of a dendritic cell or macrophage is SIRPa. In other embodiments, the 2P binds and neutralizes a ligand that binds an inhibitory receptor on a dendritic cell or macrophage. In some embodiments, the 2P binds and neutralizes a ligand that binds an inhibitory receptor on a dendritic cell or macrophage is CD47. In some embodiments, the 2P is a CD47-binding sequence of the SIRPa ECD (SEQ ID NO: 68). In some embodiments, the multi-specific polypeptide is anti-IL-23-SIRPa ECD, comprising amino acid sequences SEQ ID NO: 68 and 54. In other embodiments, the 2P binds and neutralizes a ligand that inhibits dendritic cell or macrophage maturation or function. In one embodiment, the 2P is a ligand-binding sequence of the extracellular domain of SIGLEC10.

In some embodiments, the 2P is an antagonist of a cytokine/cytokine receptor. In some embodiments, the 2P is an antagonist of immune inhibitory cytokine/cytokine receptor signaling. In some embodiments, the 2P inhibits IL-8 signaling. In some embodiments, the 2P binds IL-8.

In some embodiments, the 2P binds a ligand or cytokine that inhibits NK cell activation, maturation, or function. In some embodiments, the 2P binds a ligand or cytokine that inhibits T cell activation, maturation, or function. In some embodiments, the 2P is a ligand-binding domain of the extracellular domain of a receptor that binds such a ligand or cytokine. In one embodiment, the 2P is a ligand-binding domain of the extracellular domain of IL-10R.

In some embodiments, the 2P binds a cytokine receptor that promotes NK cell activation, maturation, or function. In some embodiments, the 2P is a cytokine or a receptor-binding fragment thereof that promotes NK cell activation, maturation, or function. In some embodiments, the 2P binds a cytokine receptor that promotes T cell activation, maturation, or function. In some embodiments, the 2P is a cytokine or a receptor-binding fragment thereof that promotes T cell activation, maturation, or function. In some embodiments, the 2P is IL-15, IL-12, IL-18, or a receptor-binding fragment thereof. In some embodiments, the 2P comprises a fusion of a receptor-binding fragment of IL-15 and a ligand-binding fragment of the IL-15R sushi domain.

In some embodiments, the 2P binds a cytokine receptor that promotes T cell activation, maturation, or function as an agonist. In some embodiments, the 2P comprises IL-2.

In some embodiments, the 2P binds an NK cell surface molecule. In some embodiments, the 2P binds an NK cell surface molecule preferentially expressed by CD56^{dim}CD16⁺ NK cells. In some embodiments, the 2P binds an NK cell surface activating receptor as an agonist. In some embodiments, the 2P is a NKG2D-binding fragment of the NKG2DL extracellular domain. The NKG2DL may be selected from MICA, MICB, or ULBP1-6.

In some embodiments, the 2P binds FGF-2 or FGFR. In other embodiments, the 2P binds PDGF or PDGFR. In other embodiments, the 2P binds angiopoietin (1, 2, 3, or 4) or an angiopoietin receptor (TIE-1 or TIE-2).

In some embodiments, the 2P inhibits the activation, differentiation, maturation, or function of TH2 cells. In some embodiments, the 2P binds IL-4, IL-13, IL4RA, or IL13R. In some embodiments, the 2P is an antibody or an antigen binding fragment thereof that binds IL4RA (e.g., dupilumab).

### Examples of 2P Moiety for Autoimmune Conditions

Multi-specific polypeptides of this invention intended for the treatment of immune disorders such as autoimmune conditions do not seek to ‘break tolerance’. Instead, effective treatment of an autoimmune disorder may involve inducing tolerance or counteracting one or more inflammatory mechanisms, in addition to blocking IL-23. As such, in some embodiments, the multi-specific polypeptides of the invention comprise a 2P as described below.

In some embodiments, the 2P localizes the fusion protein to a particular tissue. Generally, in order to mitigate NK/macrophage-mediated aggravation of the autoimmune condition, the 2P comprises an Fc-domain of human immunoglobulin. In some embodiments, the Fc domain have one or more mutations to mitigate or eliminate its binding to activating FcRs. In some embodiments, the Fc domain has one or more mutations to increase its binding to inhibitory FcRs.

In some embodiments, the 2P binds and neutralizes a pro-inflammatory cytokine. For the treatment of certain autoimmune disorders, it may be additionally advantageous for the fusion protein to neutralize an additional pro-inflammatory cytokine besides IL-23. The pro-inflammatory cytokine may be selected from IFNg, TNFα, IL-1a, IL-1b, IL-6, IL-17, IL-12, IL-18, RANKL, and GM-CSF. Exemplary such 2Ps include TNFR2-ECD (SEQ ID NO: 75) (e.g., etanercept), anti-IL17 mAb (e.g., secukinumab), RANK-ECD (SEQ ID NO: 76), or anti-GMCSF mAb (e.g., lenzilumab).

In some embodiments, the 2P binds and neutralizes a pro-inflammatory cytokine receptor. The pro-inflammatory cytokine receptor may be selected from: IFNgR, TNFR, IL-1R, IL-6R, IL-17R, IL-12R, IL-18R, RANK, and GM-CSFR.

In some embodiments, the 2P binds a T cell co-stimulatory ligand to disable its effect. In some embodiments, the 2P binds one of the following co-stimulatory ligands: CD40L, 41BBL, OX40L, ICOSL, or GITRL. In some embodiments, the 2P comprises a ligand-binding sequence of the extracellular domain of one of the following co-stimulatory receptors: CD40-ECD, 41BB-ECD, OX40-ECD, ICOS-ECD, GITR-ECD. In other embodiments, the 2P binds a T cell co-stimulatory receptor as an antagonist. In some embodiments, the 2P binds CD40, 41BB, OX40, ICOS, or GITR as an antagonist.

In some embodiments, the 2P binds a T cell co-inhibitory receptor as an agonist. In some embodiments, the 2P binds one of the following co-inhibitory receptors: PD1, BTLA, VISTA, TIGIT, LAG-3. In some embodiments, the 2P comprises a receptor-binding sequence of the extracellular domain of one of the following co-inhibitory ligands: PDL1, PDL2, HVEM.

In some embodiments, the 2P comprises a sequence of the extracellular domain of CTLA-4 (SEQ ID NO: 115) (e.g., CTLA4-Fc; abatacept).

In some embodiments, the 2P binds an inhibitory receptor on macrophages and/or dendritic cells as an agonist. In some embodiments, the 2P binds SIRPa as an agonist. In some embodiments, the 2P comprises a receptor-binding sequence of the extracellular domain of CD47.

In some embodiments, the 2P binds the receptor of an anti-inflammatory cytokine as an agonist. In some embodiments, the 2P may be the anti-inflammatory cytokine itself, or a receptor-binding fragment thereof. In other embodiments, the 2P may be an agonist antibody that binds the cytokine receptor to inhibit inflammation. In some embodiments, the anti-inflammatory cytokine receptor is selected from IL-4R, IL-10R, and TGFbR. In some embodiments, the anti-inflammatory cytokine is selected from IL-4, IL-10, and TGF-b, or a receptor-binding fragment thereof.

### Designs of Multi-Specific Polypeptides

In some embodiments, the multi-specific polypeptides of the invention are constructed as fusion proteins. In some embodiments, component parts of the fusion proteins of the invention are fused via a flexible linker. In some embodiments, the flexible linker comprises the polypeptide sequence (GGGGS) n where n is between 1 and 10. In some embodiments, a linker is used to link a 2P to the C terminus of an IIP. In some embodiments, the linker is selected from a non-cleavable linker, a peptide linker, a flexible linker, a rigid linker, a helical linker, and/or a non-helical linker. Non-limiting examples of possible linkers are disclosed in the art, for example in Chen et al. “Fusion Protein Linkers: Property, Design, and Functionality” Adv Drug Deliv Rev. 2014, 65 (10): 1357, which is incorporated herein by reference in its entirety. In some embodiments, component parts of the fusion proteins of the invention are fused without a linker between them.

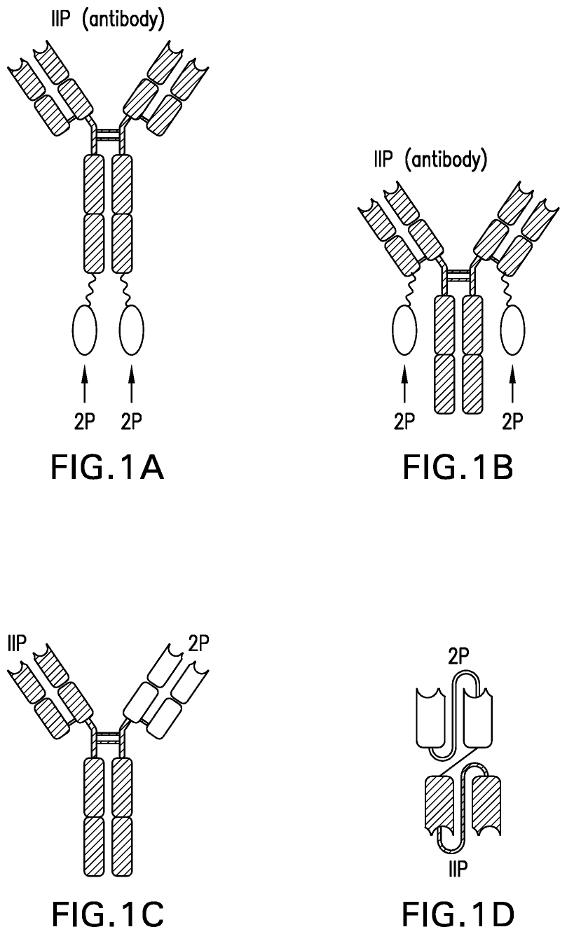

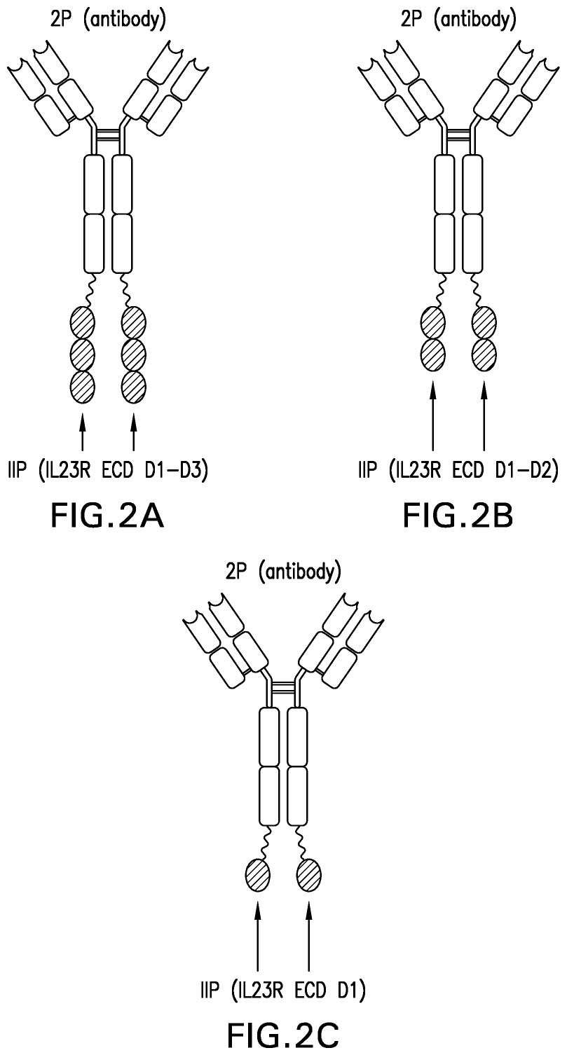

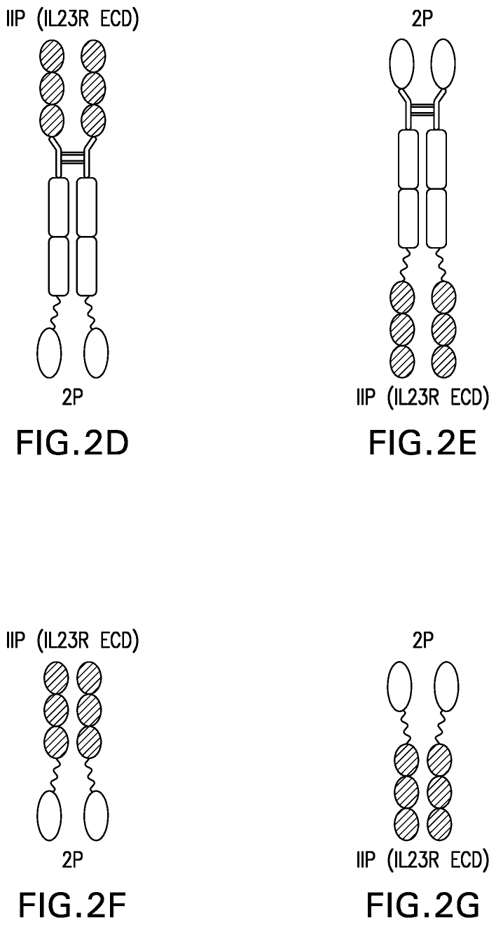

Exemplary designs of the multi-specific polypeptide used herein are depicted in, for example, FIGS. 1A-1D, 2A-2G.

In the following exemplifications, N denotes the N terminus of the protein and C denotes the C terminus of the protein. In some embodiments, the IIP is an antibody or an antigen binding fragment thereof. In such cases, the fusion protein (e.g., recombinant molecule) may have the structure of one of the following, wherein HC refers to the heavy chain of the antibody and LC refers to the light chain of the antibody:
N-HC-linker-2P-C; N-LC-C (e.g., FIG. 1A)
N-2P-linker-HC-C; N-LC-C
N-HC-C; N-LC-linker-2P-C (e.g., FIG. 1B)
N-HC-C; N-2P-linker-LC-C

In some embodiments, the molecule is a bispecific antibody wherein one Fab of the bispecific antibody is the IIP and the other Fab of the bispecific antibody is the 2P (e.g., FIG. 1C). In other embodiments, the molecule is a fusion of two scFv antibody fragments, wherein one scFv is the IIP and the other scFv is the 2P (e.g., FIG. 1D).

In other embodiments, the 2P is an antibody. In such cases, the fusion protein may have the structure of one of the following, wherein HC refers to the heavy chain of the antibody and LC refers to the light chain of the antibody:
N-HC-linker-IIP-Fc; N-LC-C (e.g., FIG. 2A-C).
N-IIP-linker-HC-Fc; N-LC-C
N-HC-C; N-LC-linker-IIP-C
N-HC-C; N-IIP-linker-LC-C

In some embodiments, the IIP and 2P moieties are fused in one of the following ways:
N-IIP-Fc-2P-C (e.g., FIG. 2D)
N-IIP-2P-C (e.g., FIG. 2F)
N-IIP-Fc-linker-2P-C
N-IIP-linker-2P-C.
N-2P-Fc-IIP-C (e.g., FIG. 2E)
N-2P-IIP-C (e.g., FIG. 2G)
N-2P-Fc-linker-IIP-C
N-2P-linker-IIP-C

In some embodiments, the multi-specific polypeptide of the invention is a bispecific antibody (bsAb). In some embodiments, the bSab is an obligate or non-obligate bsAb.

In some embodiments, the bsAb is bivalent in a 1+1 format (i.e., one binding site for each target). In a further embodiment, the bispecific antibody may be a tandem VHH nanobody fusion, tandem scFvs (e.g., BiTE), DART, diabody, F(ab)2, or scFv-Fab fusion. In another embodiment, the bispecific antibody may comprise two or more asymmetric chains: for example, hetero heavy chains with forced knob-and-hole HL pairing, hetero heavy chains with CrossMab VH/VL swapped domains, hetero heavy chains with CrossMAB CH1/CL swapped domains, DART-Fc, LP-DART, or half-life-extended BiTE.

In other embodiments, the bsAb is trivalent in a 1+2 format (i.e., 1 binding site for one target and 2 binding sites for the other target). In a further embodiment, the bsAb is a CrossMab with 3 F(ab) regions.

In other embodiments, the bsAb is tetravalent in a 2+2 format (i.e., 2 binding sites for each target). In a further embodiment, the bsAb is a fusion of a normal IgG with 2 scFv domains, Bs4Ab, DVD-Ig, tetravalent DART-Fc, four scFv domains fused to Fc, CODV-Ig, a pair of tandem VHH nanobodies fused to Fc, or a CrossMab with 4 F(ab) regions.

In some embodiments, the bsAb comprises the VH and VL of any one of risankizumab, guselkumab, tildrakizumab, brazikumab, mirikizumab. In some embodiments, the bsAb further comprises the VH and VL of another antibody or antigen-binding fragment thereof.

In some embodiments, the bsAb comprises one or more of the six complementarity-determining regions (CDRs) of any one of risankizumab, guselkumab, tildrakizumab, brazikumab, mirikizumab. In some embodiments, the bsAb further comprises additional CDRs of another antibody or antigen-binding fragment thereof.

### Exemplary Multi-Specific Polypeptides

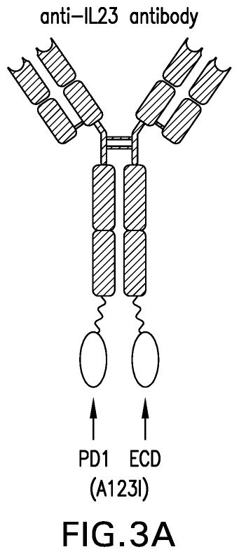

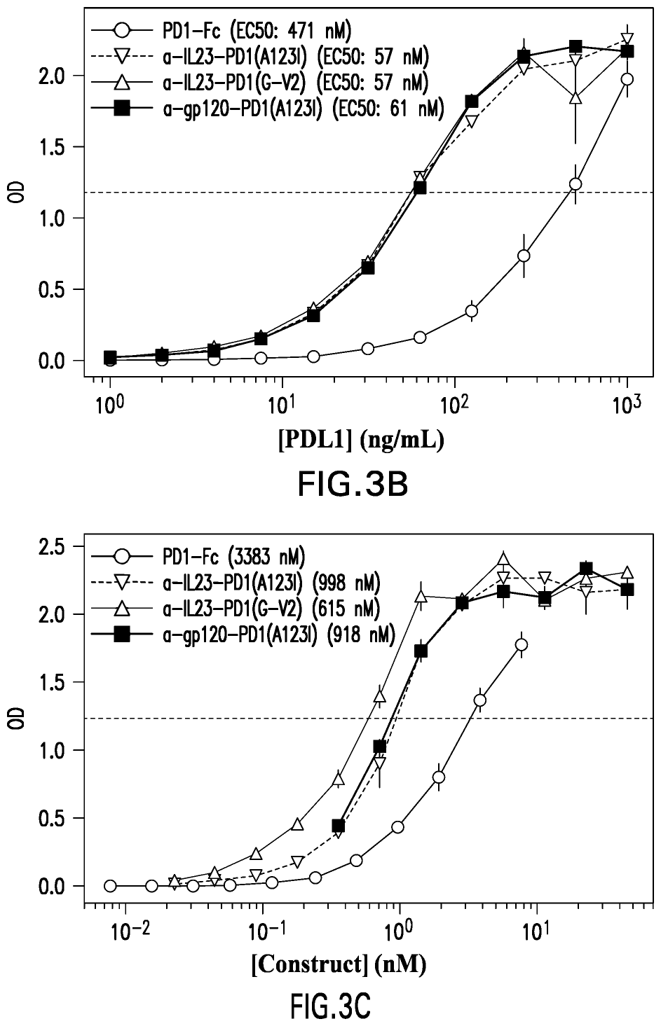

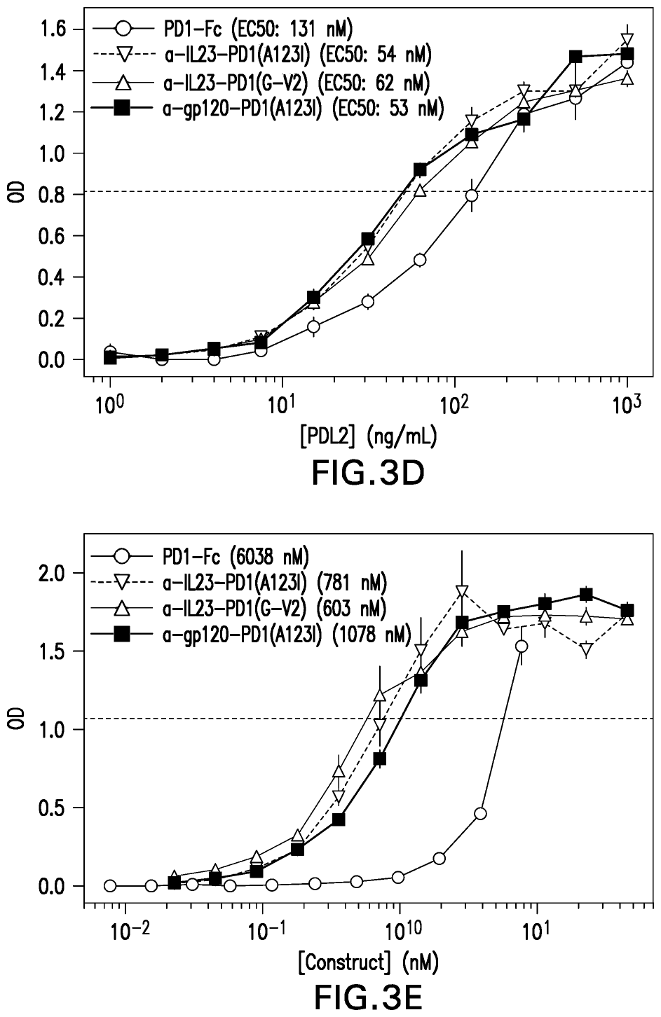

As schematically depicted in FIGS. 1A and 3A, anti-IL-23-PD1ecd is an exemplary molecule having an anti-IL-23 antibody (IIP) fused or linked to a ligand-binding sequence of the PD1 ECD (2P) at the C-terminus of its heavy chain via the flexible linker (GGGGS) 3. The PD1 ECD comprises the A132I mutation. The sequence for this exemplary molecule is given in SEQ ID NO: 53 and SEQ ID NO: 54.

In certain embodiments recombinant molecules described herein can include “conservative sequence modifications” of any of the sequences set forth in SEQ ID NOs: 1-116, i.e., nucleotide and amino acid sequence modifications which do not abrogate the binding of the VH and VL sequences encoded by the nucleotide sequence or containing the amino acid sequence, to the antigen. Such conservative sequence modifications include conservative nucleotide and amino acid substitutions, as well as nucleotide and amino acid additions and deletions. For example, modifications can be introduced into SEQ ID NOs: 1-116 by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in any of the moieties described herein can be replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12 (10): 879-884 (1999); and Burks et al. Proc. Natl. Acad. Sci. USA 94:412-417 (1997)).

In certain embodiments, conservative amino acid sequence modifications refer to at most 1, 2, 3, 4 or 5 conservative amino acid substitutions to the CDR sequences described herein. For example, each such CDR may contain up to 5 conservative amino acid substitutions, e.g., up to (i.e., not more than) 4 conservative amino acid substitutions, e.g., up to (i.e., not more than) 3 conservative amino acid substitutions, e.g., up to (i.e., not more than) 2 conservative amino acid substitutions, or no more than 1 conservative amino acid substitution.

In some embodiments, the multi-specific polypeptide of the invention comprises an IIP that is an antibody and a 2P that is a polypeptide fused to the C terminus of the heavy chain of the IIP antibody. Exemplary fusion proteins (using anti-IL-23 antibody guselkumab as the IIP) are provided as the following: heavy chain corresponding to any one of SEQ ID NOs. 53 or 97-109; and light chain corresponding to SEQ ID NO: 54.

In some embodiments, the IIP is an antibody comprising one of the following VH/VL pairs: SEQ ID NOs. 79+80, SEQ ID NOs. 81+82, SEQ ID NOs. 83+84, SEQ ID NOs. 85+86, or SEQ ID NOs. 87+88. The IIP antibody may further comprise an IgG1 constant region fused to the C terminus of the VH, which may be selected from SEQ ID NO: 111-112. The The IIP antibody may further comprise a light chain constant region fused to the C terminus of the VL. The IIP antibody may be fused to the 2P moiety, either with or without a linker which may be SEQ ID NO: 55. The 2P may be selected from SEQ ID NOs. 56-78 or 89.

### Design of Recombinant IL-23R-ECD

In various embodiments, exemplary designs of IL-23R-ECD are as follows: IL-23R_{D1D2D3} (SEQ ID NO: 6), IL-23R_{D1D2} (SEQ ID NO: 5), or IL-23R_{D1} (SEQ ID NO: 2).

In some embodiments, the IIP of the multispecific polypeptide of the invention may comprise a ligand-binding domain of the extracellular domain of human IL-23R, and a ligand-binding domain of the extracellular domain of human IL-12Rb (IL-23R/IL12R-ECD).

In order to signal, native IL-23R expressed on the cell surface binds the IL-23 heterodimer (p19, p40). This heterotrimer generally binds IL-12Rb to activate IL-23R signaling. Similarly, native IL 12Ra expressed on the cell surface binds the IL-12 heterodimer (p35, p40). This heterotrimer then binds the same IL-12Rb to activate IL 12R signaling.

Without being bound to any theories, it is possible that IL-23R-ECD of the fusion protein binds a complete IL-23 heterodimer (p19, p40) and this heterotrimer of IL-23R/p19/p40 is able to bind native IL-12Rb. This decreases the number of IL 12Rb subunits available for IL 12R signaling and as such, may lead to a decrease of IL 12R signaling. In the case of treating cancer, this may be undesirable. Accordingly, described herein are the design of a chimeric IL-23R-ECD-IL 12Rb-ECD fusion (IL-23R/IL 12R-ECD) to prevent this undesirable consequence of sequestration of native IL12Rb.

IL-23R/IL12R-ECD comprises a ligand-binding sequence of IL-23R that binds p19 and a ligand-binding sequence of IL-12Rb that binds p40.

IL-23R is composed of an N-terminal Ig-like domain (D1), two fibronectin type III domains (D2 and D3), followed by a stalk region, transmembrane domain, and cytoplasmic domain. IL 12Rb starts with two N-terminal fibronectin type III domains (D1, D2) followed by three fibronectin type III-like domains (D3, D4, D5), followed by a transmembrane domain and cytoplasmic domain.

In some embodiments, IL-23R/IL 12R-ECD comprises one or more domains of IL-23R selected from: D1, D2, D3. In some embodiments, IL-23R/IL12R-ECD comprises one or more domains of IL12Rb selected from: D1, D2. In some embodiments, IL-23R/IL12R-ECD may comprise IL-23R_{D1}, IL-23R_{D1D2}, or IL-23R_{D1D2D3}. In some embodiments, IL-23R/IL12R-ECD may comprise IL 12Rb_{D1}, IL12Rb_{D2}, or IL12Rb_{D1D2}.

In some embodiments, the IL 12Rb domains and the IL-23R domains are fused or linked via a flexible linker. In some embodiments, IL-23R/IL12R-ECD has the form N-IL-23R domain(s)-linker-IL12R domain(s)-C. In other embodiments, IL-23R/IL12R-ECD has the form N-IL12R domain(s)-linker-IL-23R domain(s)-C. In a further aspect, the flexible linker comprises the polypeptide sequence (GGGGS) n where n is between 1 and 10.

Exemplary embodiments of IL-23R/IL12R-ECD are as follows: IL12Rb_{D1}-linker-IL-23R_{D1} (SEQ ID NO: 29), IL12Rb_{D1}-linker-IL-23R_{D1D2} (SEQ ID NO: 30), IL12Rb_{D1}-linker-IL-23R_{D1D2D3} (SEQ ID NO: 31), IL12Rb_{D1D2}-linker-IL-23R_{D1} (SEQ ID NO: 32), IL12Rb_{D1D2}-linker-IL-23R_{D1D2} (SEQ ID NO: 33), IL-23R_{D1}-linker-IL 12Rb_{D1} (SEQ ID NO: 34), IL-23R_{D1D2}-linker-IL12Rb_{D1} (SEQ ID NO: 35), IL-23R_{D1D2D3}-linker-IL12Rb_{D1} (SEQ ID NO: 36), IL-23R_{D1}-linker-IL12Rb_{D1D2} (SEQ ID NO: 37), or IL-23R_{D1D2}-linker-IL 12Rb_{D1D2} (SEQ ID NO: 38).

Exemplary embodiments of the fusion protein include: anti-CEA antibody with IL-23R_{D1} fused to C terminus of HC (SEQ ID NO: 12, 13), anti-CEA antibody with IL-23R_{D1} fused to C terminus of LC (SEQ ID NO: 11, 14), anti-CEA antibody with IL-23R_{D1D2} fused to C terminus of HC (SEQ ID NO: 12, 15), anti-CEA antibody with IL-23R_{D1D2} fused to C terminus of LC (SEQ ID NO: 11, 16), anti-CEA antibody with IL-23R_{D1D2D3} fused to C terminus of HC (SEQ ID NO: 12, 17), anti-CEA antibody with IL-23R_{D1D2D3} fused to C terminus of LC (SEQ ID NO: 11, 18), anti-CEA antibody with IL-23R_{D1} fused to C terminus of LC and TGFbRII-ECD fused to C terminus of HC (SEQ ID NO: 14, 19), TNFR-ECD-Fc-IL-23R_{D1} (SEQ ID NO: 20), TNFR-ECD-Fc-IL-23R_{D2} (SEQ ID NO: 21), TNFR-ECD-Fc-IL-23R_{D1D2D3} (SEQ ID NO: 22), Fc-IL-23R_{D1} (SEQ ID NO: 23), Fc-IL-23R_{D1D2} (SEQ ID NO: 24), Fc-IL-23R_{D1D2D3} (SEQ ID NO: 25), IL-23R_{D1}-Fc (SEQ ID NO: 26), IL-23R_{D1D2}-Fc (SEQ ID NO: 27), IL-23R_{D1D2D3}-Fc (SEQ ID NO: 28), anti-CEA antibody with IL12Rb_{D1}-linker-IL-23R_{D1} fused to C terminus of HC (SEQ ID NO: 39), anti-CEA antibody with IL12Rb_{D1}-linker-IL-23R_{D1} fused to C terminus of LC (SEQ ID NO: 40), VEGFR-Fc-IL-23R_{D1} (SEQ ID NO: 41), IL-23R_{D1}-Fc-VEGFR (SEQ ID NO: 42), TGFbRII-Fc-IL-23R_{D1} (SEQ ID NO: 43), IL-23R_{D1}-Fc-TGFbRII (SEQ ID NO: 44), PSMA-binding peptide fused to Fc and IL-23R_{D1} (SEQ ID NO: 45), anti-PSMA antibody with IL-23R_{D1} fused to C terminus of HC (SEQ ID NO: 48, 47), anti-PSMA antibody with IL-23R_{D1} fused to C terminus of LC (SEQ ID NO: 46, 49), anti-PSMA antibody with IL-23R_{D1} fused to C terminus of LC and TGFbRII-ECD fused to C terminus of HC (SEQ ID NO: 50, 49), or anti-IL-23 antibody with TGFbRII-ECD fused to C terminus of HC and PSMA-binding peptide fused to C terminus of LC (SEQ ID NO: 51, 52).

### Combination Therapies for Treating of a Cancer

Provided herein are also methods of treating a neoplastic disease or a cancer in a subject, comprising administering to the subject an effective amount of pharmaceutical composition(s) comprising one or more therapeutic agents, wherein the one or more therapeutic agents comprises at least a first therapeutic agent comprising an inhibitor of IL-23/IL-23R signaling (“a-IL-23 agent”); and a second therapeutic agent (“combination agent”). The second therapeutic agent may comprise an antagonist of one or more immune checkpoint proteins; an agonist of one or more immune stimulatory receptors; an antagonist of the signaling of one or more cytokines; an agonist of one or more cytokine receptors; a modulator of one or more cell surface molecules expressed or displayed on the cell surface of a tumor cell or an immune cell; immune cells comprising CAR-T cells, CAR-NK cells, or hematopoietic stem cells; an immunogenic chemotherapeutic agent; and/or an antagonist of one or more immune inhibitory enzymes.

In some embodiments, the a-IL-23 agent comprises an antibody that binds IL-23p19 (e.g., risankizumab, guselkumab, tildrakizumab, brazikumab, mirikizumab). In other embodiments, the a-IL-23 agent is an antibody that binds IL-23R (e.g., AS2762900-00). In other embodiments, the a-IL-23 agent comprises a fusion protein comprising an antibody that binds IL-23p19 or IL-23R. In other embodiments, the a-IL-23 agent is a multi-specific polypeptide/fusion protein of this invention. In some aspects, the a-IL-23 agent is an antibody-ligand trap fusion protein comprising IL-23R-ECD.

In some embodiments, the combination agent inhibits TGFb/TGFbR. In some embodiments, the TGFb/TGFbR inhibitor is selected from the following: a-TGFb antibody (e.g., fresolimumab); a-TGFbR antibody; TGFbRII ECD containing fusion protein (e.g. TGFbRIIecd-Fc, AVID200); TGFbR TKI (e.g. galunisertib); anti-GARP antibody; anti-LAP antibody; fusion proteins comprising an antibody and TGFbRII ECD (e.g., a-PDL1-TGFbRIIecd; bintrafusp alfa, SIRPa ECD-TGFbRII, anti-CEA-TGFbRII, anti-PSMA-TGFbRII, anti-IL6R-TGFbRII, anti-PD1-TGFbRII, anti-EGFR-TGFbRII, or anti-HER2-TGFbRII). In a specific embodiment, the combination agent is anti-EGFR-TGFbRII. In a specific embodiment, the combination agent is BCA101.

In some embodiments, the combination agent inhibits VEGF/VEGFR. In some embodiments, the VEGF/VEGFR inhibitor may be selected from: anti-VEGF antibody (e.g., bevacizumab), anti-VEGFR antibody (e.g. ramucirumab), VEGFR kinase inhibitor (e.g., sunitinib, sorafenib, axitinib, cabozantinib, regorafenib, pazopanib, vandetanib, lenvatenib), VEGFR ECD-Fc fusion protein (e.g., aflibercept), or fusion proteins comprising an antibody and VEGFR ECD.

In some embodiments, the combination agent inhibits the interaction of CD47 and SIRPa. In some aspects, the CD47/SIRPa inhibitor may be selected from: a-CD47 mAb (e.g., magrolimab, ZL-1201, TJ011133, STI-6643, SRF231, SHR-1603, IMC-002, IBI188, CC-90002, AO-176, or AK117, letaplimab, urabrelimab), a-SIRPa mAb, SIRPa-ECD containing fusion protein (e.g., SIRPa-Fc, evorpacept, TTI-621, TTI-622).

In some embodiments, the combination agent inhibits the interaction of SIGLEC10 and CD24.

In some embodiments, the combination agent is an immune checkpoint inhibitor. In some embodiments, the combination agent is an antagonist of an innate immune checkpoint receptor or ligand. In some embodiments, the combination agent is an antagonist of a T cell co-inhibitory molecule. In some embodiments, the combination agent inhibits the interaction of PD-1 and PD-L1 or PD-L2. In some embodiments, the combination agent is an antibody that binds PD-1 (e.g., nivolumab, pembrolizumab, cemiplimab, dostarlimab, spartalizumab, camrelizumab, sintilimab, sasanlimab, tiselizumab, or toripalimab) or PDL1 (e.g., durvalumab, avelumab, atezolizumab). In other embodiments, the combination agent inhibits the interaction of BTLA and HVEM. In other embodiments, the combination agent inhibits the interaction of TIGIT and PVR. In some embodiments, the combination agent inhibiting TIGIT is selected from tiragolumab, vibostolimab, BMS-986207, ociperlimab, etigilimab, domvanalimab, EOS-448, SEA-TGT, ASP8374, COM902, or IBI939. In other embodiments, the combination agent inhibits the interaction of TIM-3 and CEACAM. In some embodiments, the combination agent inhibits LAG-3. In some embodiments, the combination agent inhibiting LAG-3 is selected from relatlimab, fianlimab, Sym022, GSK2831781, TSR-033, iermilimab, favezelimab, tebotelimab, FS118, or pavunalimab.

In some embodiments, the combination agent is an agonist of an immune stimulatory receptor. In some embodiments, the combination agent is an agonist of a T cell co-stimulatory molecule. In some embodiments, the combination agent is a polypeptide comprising the corresponding co-stimulatory ligand or receptor-binding fragment thereof. In other embodiments, the combination agent is an agonist antibody that binds a T cell co-stimulatory receptor. In some embodiments, the combination agent binds 4-1BB (CD137), Inducible T-Cell Costimulator (ICOS), OX-40 (CD134), Herpesvirus Entry Mediator (HVEM), glucocorticoid-induced TNFR-related protein (GITR), CD40, CD30, DNAM, or CD27. In some embodiments, the combination agent is a fusion protein comprising a receptor-binding sequence of the extracellular domain of CD30L, 4-1BBL, BTLA, LIGHT, OX-40L, ICOS-L, GITRL, CD80, CD86, or CD40L. In some embodiments, the combination agent is FPT-155. In some embodiments, the combination agent comprises an antibody or antigen-binding fragment thereof that binds 4-1BB as an agonist (e.g., urelumab, utomilumab). In some embodiments, the combination agent comprises an antibody or antigen-binding fragment thereof that binds OX40 as an agonist (e.g., tavolimab, PF-04518600, BMS-986178, MOXR-0916, GSK-3174998, INCAGN01949). In some embodiments, the combination agent comprises an antibody or antigen-binding fragment thereof that binds ICOS as an agonist (e.g., GSK-3359609, JTX-2011). In some embodiments, the combination aTgent comprises an antibody or antigen-binding fragment thereof that binds GITR as an agonist (e.g., TRX-518, MK-4166, MK-1248, GWN-323, INCAGN01876, BMS-986156, AMG-228). In some embodiments, the combination agent comprises an antibody or antigen-binding fragment thereof that binds CD40 as an agonist (e.g., CDX-1140, SEA-CD40, RO7009789, JNJ-64457107, APX-005M, Chi Lob 7/4). In some embodiments, the combination agent comprises an antibody or antigen-binding fragment thereof that binds CD27 as an agonist (e.g., varlilumab). In some embodiments, the combination agent binds a TNFR superfamily member receptor as an agonist.

In some embodiments, the combination agent is an agonist of an immune stimulatory receptor expressed on innate immune cells. In some embodiments, the combination agent is an agonist of an immune stimulatory receptor expressed on NK cells. In some embodiments, the NK cell immune stimulatory receptor is NKG2D. In some embodiments, the combination agent is a polypeptide comprising a NKG2D-binding fragment of an NKG2D ligand (NKG2DL).

In some embodiments, the combination agent is a tumor-targeted antibody. In some embodiments, the combination agent binds a tumor cell surface molecule, tumor antigen, or tumor-associated antigen. In some embodiments, the tumor-targeted antibody has an Fc domain that binds activating receptors on NK cells and/or macrophages (e.g., FcgRI, FcgRIII). In some embodiments, the Fc domain of the tumor-targeted antibody has mutations designed to increase its binding to one or more Fc receptors.

In some embodiments, the combination agent is a cytokine that activates NK cells, or a fusion protein comprising a cytokine that activates NK cells. In some embodiments, this cytokine may be IL-15, IL-12, or IL-18. In some embodiments, the combination agent may be ST-067, nogapendekin alfa, SHR1501, BJ-001, SO-C101 or NHS-IL12. In some embodiments, the combination agent is a virus or plasmid encoding a cytokine.

In some embodiments, the combination agent is a hormonal treatment. In some embodiments, the hormonal agent inhibits androgen synthesis or inhibits androgen receptor signaling. In some embodiments, the hormonal agent is an LHRH agonist (e.g., goserelin, histrelin, leuprolide, or triptorelin); LHRH antagonist (e.g., degarelix), first-generation antiandrogen (e.g., nilutamide, flutamide, or bicalutamide), second-generation antiandrogen (e.g., apalutamide, enzalutamide, or darolutamide), or androgen synthesis inhibitor (e.g., abiraterone acetate).

In some embodiments, the combination agent is a cytotoxic agent. In some embodiments, the combination agent is a chemotherapeutic agent, radiation, or tumor-targeted antibody.

In some embodiments, the combination agent is an antibody-drug conjugate. In some embodiments, the combination agent is selected from the following list: gemtuzumab ozogamicin, brentuximab vedotin, trastuzumab emtansine, inotuzumab ozogamicin, polatuzumab vedotin, enfortumab vedotin, trastuzumab deruxtecan, belantamab mafodotin, or sacituzumab govitecan.

In some embodiments, the combination agent is a small-molecule kinase inhibitor. In some embodiments, the combination agent is a PARP inhibitor. In some embodiments, the combination agent is a tumor vaccine or viriolytic agent. In some embodiments, the combination agent is an inhibitor of T_{H}17 differentiation, maintenance, or function. In some embodiments, the combination agent inhibits IL-17/IL-17R, IL-6/IL-6R, or IL-1/IL-1R. In some embodiments, the combination agent is an anti-IL6 antibody or anti-IL6R antibody.

In some embodiments, the combination agent is an antagonist of the signaling of one or more cytokines. In some embodiments, the cytokine is an immune inhibitory cytokine. In some embodiments, the cytokine is selected from the following: IL-4, IL-13, IL-10, IL-6, IL-1b, IL-17, IL-22. In some embodiments, the combination agent is a polypeptide that binds the cytokine. In other embodiments, the combination agent is a polypeptide that binds the cytokine's cognate cytokine receptor. In some embodiments, the combination agent binds and inhibits IL 1b or IL1R. In some embodiments, the combination agent is selected from anakinra or canakinumab. In some embodiments, the combination agent binds and inhibits IL-10 or IL-10R. In some embodiments, the combination agent is an antibody or antigen binding fragment thereof that binds IL-10 or IL-10R; or a polypeptide comprising an IL10-binding fragment of IL-10R. In some embodiments, the combination agent is an antibody thata binds IL-17 or IL-17R. In some embodiments, the combination agent is selected from afasevikumab, bimekizumab, ixekizumab, netakimab, perakizumab, secukinumab, vunakizumab, or brodalumab. In some embodiments, the combination agent is an antibody that binds IL-6 or IL-6R. In some embodiments, the combination agent is selected from clazakizumab, olokizumab, siltuximab, sirukumab, ziltivekimab, levilimab, sapelizumab, sarilumab, satralizumab, or tocilizumab. In some embodiments, the combination agent is an antibody that binds IL-4, IL-13, IL4RA, or IL13R. In some embodiments, the combination agent is dupilumab.

In some embodiments, the combination agent is an antagonist of RANK/RANKL signaling. In some embodiments, the combination agent is an antibody that binds RANKL or RANK. In some embodiments, the combination agent is denosumab. In other embodiments, the combination agent is a polypeptide comprising a RANKL-binding fragment of the RANK ECD.

In some embodiments, the combination agent comprises one or more agents selected from the following: immunotherapeutic agent, chemotherapeutic molecule, antibody, antibody-drug conjugate, small molecule kinase inhibitor, hormonal agent, androgen synthesis inhibitor, androgen receptor antagonist, anti-angiogenic agent, cell therapy, CAR-T cellular therapy, CAR-NK cellular therapy, radionuclide therapy, ionizing radiation, ultraviolet radiation, cryoablation, thermal ablation, a selective estrogen receptor modulator (SERM), a selective estrogen receptor degrader (SERD), or radiofrequency ablation. In some embodiments, the immunotherapeutic agent is selected from the following: immune checkpoint inhibitor, immune stimulatory receptor agonist, immune stimulatory cytokine/cytokine receptor agonist, immune inhibitory cytokine/cytokine receptor antagonist, tumor vaccine, immunomodulatory imide drug, CAR-T cells, CAR-NK cells, oncolytic virus.

In some embodiments, the combination agent is an immunogenic chemotherapeutic agent. The mechanism of action of immunogenic chemotherapy may involve immune activation, and as such, may be hindered by expansion/activation of IL-23-dependent inflammatory cells. In some embodiments, the immunogenic chemotherapeutic agent is an alkylating agent, topoisomerase inhibitor, platinum derivative, taxane, or anthracycline.

In some embodiments, the combination agent is an antagonist of immune inhibitory enzymes. In some embodiments, the immune inhibitory enzyme is an ectonucleotidase (e.g., CD39, CD73) or indoleamine 2,3-dioxygenase.

In a tumor immune microenvironment enriched with IL-23, engineered T cells or NK cells adoptively transferred into a patient (CAR-T, CAR-NK cells, respectively) may adopt an undesirable, tumor-promoting phenotype. In some embodiments, the combination agent is a composition comprising CAR-T cells or CAR-NK cells.

In some embodiments, the cancer is a hematological or hematogenous cancer selected from the group consisting of acute leukemia, acute myelocytic leukemia, acute myelogenous leukemia, myeloblastic leukemia, promyelocytic leukemia, myelomonocytic leukemia, monocytic leukemia, erythro leukemia, chronic leukemia, chronic myelocytic (or granulocytic) leukemia, chronic myelogenous leukemia, chronic lymphocytic leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia, and any combination thereof.

In some embodiments, the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In some embodiments, the method of treatment comprises administration of a multi-specific polypeptide of the invention comprising a 2P that comprises a polypeptide sequence that binds TfR and thereby crosses the blood-brain barrier. In some such embodiments, the cancer is a CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In some embodiments, the method comprises treatment with the a-IL-23 agent concurrently or sequentially with the combination agent. For instance, treatment with the a-IL-23 agent may be administered at the same time (e.g., in the same pharmaceutical composition, or within a time frame from between about 0.1 hour to about 24 hours) of administration of the combination agent; treatment with the a-IL-23 agent may be administered 1-28 days following administration of the combination agent; or treatment with the a-IL-23 agent may be administered 1-28 days before administration of the combination agent.

In some embodiments, the treatment with either a single agent or combination is repeated periodically for time frames of from once every month, to once every two months, to once every 3 months, to once every 4 months, to once every 5 months, to once every 6 months, or once every 7 months, or once every 8 months, or once every 9 months, or once every 10 months, or once every 11 months, or once annually as a maintenance treatment, for as long as the patient exhibits improvement or stable/non-progressing disease.

In some embodiments, the method of treatment with the combination of the anti-IL-23 agent and the other anti-cancer agent(s) reduces the incidence and/or severity of immune-related adverse events (irAEs) compared to treatment with the other anti-cancer agent(s) alone. In some embodiments, the reduction in the percentage of patients who discontinue therapy due to toxicity is at least 10%, 30%, 50%, 70%, or 90%. In some embodiments, the reduction in incidence of grade 3, grade 4, grade 3+4, or all grade irAEs is at least 10%, 30%, 50%, 70%, or 90%. In some embodiments, the reduction of grade 3, grade 4, grade 3+4, or all grade of a particular class of irAEs is at least 10%, 30%, 50%, 70%, or 90%; wherein the classes may, without limitation, be selected from gastrointestinal, endocrine, cardiac, pulmonary, hepatic, rheumatalogical, renal, neurological or dermatologic/cutaneous. In some embodiments, the reduction of grade 3, grade 4, grade 3+4, or all grade of a particular irAE is at least 10%, 30%, 50%, 70%, or 90%; wherein the particular irAEs may, without limitation, be selected from uveitis, Sjogren syndrome, conjunctivitis, blepharitis, episcleritis, scleritis, retinitis, pneumonitis, pleuritis, sarcoid-like granulamatosis, hepatitis, pancreatitis, autoimmune diabetes, skin rash, pruritus, vitiligo, DRESS, psoriasis, Stevens-Johnson syndrome, arthralgia, arthritis, myositis, dermatomyositis, encephalitis, meningitis, polyneuropathy, fatigue, Guillain-Barré syndrome, hypophysitis, thyroiditis, adrenalitis, myocarditis, pericarditis, interstitial nephritis, glomerulonephritis, colitis, enteritis, gastritis, anaemia, neutropenia, thrombocytopenia, thrombotic microangiopathy, acquired haemophilia, vasculitis, or any Common Terminology Criteria for Adverse Events (CTCAE) adverse event.

In some embodiments, the method of treatment with the combination of the anti-IL-23 agent and the other anti-cancer agent(s) prolongs overall survival or progression-free survival more effectively than treatment with the other anti-cancer agent(s) alone. In some embodiments, the method of treatment with the combination of the anti-IL-23 agent and the other anti-cancer agent(s) results in a statistically significant improvement in any RECIST v1.1 criteria, as is well-described in the art; for example, in Eisenhauer et al., “New response evaluation criteria in solid tumours: Revised RECIST guideline (version 1.1)” European Journal of Cancer 2009; 45:228, which is incorporated herein in its entirety.

In some embodiments, the method of treatment with the combination of the anti-IL-23 agent and the other anti-cancer agent(s) reduces or prevents bone metastases or skeletal-related events, more effectively than treatment with the other anti-cancer agent(s) alone. In some embodiments, the reduction in bone metastases is as per RECIST v1.1 criteria. In some embodiments, the reduction in skeletal-related events is at least 10%, 30%, 50%, 70%, or 90%.

In some embodiments, the method of treatment with the combination of the anti-IL-23 agent and the other anti-cancer agent(s) results in an improvement in both efficacy and toxicity as described above compared to treatment with the other anti-cancer agent(s) alone.

In some embodiments, the treatment is repeated periodically for time frames of from once every two weeks, to once every three weeks, to once every month, to once every two months, to once every 3 months, to once every 4 months, to once every 5 months, to once every 6 months, or once every 7 months, or once every 8 months, or once every 9 months, or once every 10 months, or once every 11 months, or once annually as a maintenance treatment, for as long as the patient exhibits improvement or stable/non-progressing disease.

In some embodiments, the treatment prevents metastasis, inhibits tumor growth, and/or reduces tumor growth.

### Therapeutics for Treating an Immune Disorder or an Autoimmune Condition

Provided herein are methods of treating an immune disorder in a subject, comprising administering to the subject an effective amount of a pharmaceutical composition comprising one or more therapeutic agents, wherein the one or more therapeutic agents comprises an anti-IL-23 agent, or multi-specific polypeptide of this invention.

In some embodiments, the method further comprises a second agent. In some embodiments, this second agent is an agonist of IL10 signaling. In some embodiments, the second agent comprises an agonist IL10R antibody or an IL10R-binding sequence of IL10.

In some embodiments, the method of treatment comprises administration of a multi-specific polypeptide of the invention comprising a 2P that comprises a polypeptide sequence that binds TfR and thereby crosses the blood-brain barrier. In some such embodiments, the immune disorder is multiple sclerosis or causes neuroinflammation.

In some embodiments, the immune disorder is an autoimmune disorder. As referred to herein, non-limiting examples of immune disorders include Addison disease, celiac disease, dermatomyositis, graves disease, Hashimoto thyroiditis, multiple sclerosis, myasthenia gravis, pernicious anemia, reactive arthritis, rheumatoid arthritis, Sjogren's syndrome, scleroderma, systemic sclerosis, systemic lupus erythematosus, or type I diabetes, chronic inflammatory diseases, psoriasis, ulcerative colitis, Crohn's disease, and inflammatory bowel diseases. In some embodiments, the immune disorder is graft versus host disease (GVHD).

In one embodiment, the invention discloses a method of treatment or prophylaxis of acute or chronic graft versus host disease comprising an anti-IL-23 agent or multi-specific polypeptide of this invention.

### Kits and Articles of Manufacture

Further provided are kits, unit dosages, and articles of manufacture comprising any of the recombinant molecules described herein. In some embodiments, a kit is provided comprising any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

### Example 1: Multifunctional Construct Designs

The multifunctional fusion protein may comprise an IIP that is an antibody or antigen-binding fragment thereof. Examples of such constructs are given in FIGS. 1A-1D.

FIGS. 1A-1D show schematics corresponding to multiple exemplary designs of multi-specific polypeptides described herein, in which the IIP (dark grey) comprises an antibody or antibody fragment, and 2P (light grey) denotes a target binding polypeptide. FIG. 1A shows an antibody IIP with a 2P fused to the C terminus of the heavy chain. FIG. 1B shows an antibody IIP with a 2P fused to the C terminus of the light chain. FIG. 1C shows a bispecific antibody wherein one Fab is the IIP and the other Fab is the 2P. FIG. 1D shows a fusion of two scFvs wherein one scFv is the IIP and the other scFv is the 2P.

FIGS. 2A-2G show schematics corresponding to multiple exemplary designs of multi-specific polypeptides described herein, in which the IIP comprises a ligand-binding fragment of IL-23R ECD. D1, D2 and D3 denote the IL-23R binding domains 1, 2, and 3, respectively. In these figures, dark grey corresponds to the IIP and light grey The multifunctional fusion protein may comprise an IIP that comprises a ligand-binding fragment of the IL-23 ECD.

The amino acid sequences of exemplary fusion proteins of the invention were codon optimized with GeneOptimizer®. The cDNA for the antibody heavy chain and the cDNA for the antibody light chain were synthesized and subsequently cloned into separate plasmids (pEvi3; evitria AG, Switzerland) under the control of a mammalian promoter and polyadenylation signal. Plasmid DNA was amplified in E. coli and DNA was purified using anion exchange kits for low endotoxin plasmid DNA preparation. DNA concentration was determined by measuring the absorption at a wavelength of 260 nm. Correctness of the sequences was verified with Sanger sequencing (with up to two sequencing reactions per plasmid depending on the size of the cDNA.) The plasmid DNAs for heavy and light chain were subsequently co-transfected into suspension-adapted CHO K1 cells (originally received from ATCC and adapted to serum-free growth in suspension culture at evitria). The seed was grown in eviGrow medium, a chemically defined, animal-component free, serum-free medium. Cells were transfected with eviFect (evitria AG, Switzerland). and the CHO cells were cultured in eviMake2 (evitria AG, Switzerland), a serum-free, animal-component free medium. Production was terminated once viability reached 75%, which occurred at day 8 after transfection. Supernatant was harvested by centrifugation and subsequent filtration (0.2 um filter). The antibody was purified using MabSelect™ Sure™ (Protein A affinity chromatography on a Bio-Rad BioLogic FuoFlow FPLC machine with subsequent gel filtration as polishing and rebuffering step). In some cases, the antibody was further purified using SEC purification.

The fusion proteins of the invention can also be produced via stable transfection of a mammalian cell line (e.g. CHO K1 cells) with plasmid DNA encoding the chains of the fusion protein, selection of stably transfected cell clones or cell pools expressing the fusion protein, development of a Master Cell Bank for production of the fusion protein, purification of the fusion protein by Protein A affinity chromatography and/or SEC, and formulation using methods well described in the art.

### Example 2: Anti-IL-23-PD1ecd Construct with a Single Amino Acid Mutation in the PD1ecd Exhibits Superior Binding Affinity for PDL1

As shown in FIGS. 3A-3D, data demonstrated that the anti-IL-23-PD1ecd construct with a single amino acid mutation in the PD1ecd exhibits superior binding affinity for PDL1 (FIGS. 3B-3C) and PDL2 (FIGS. 3D-3E) compared to wild-type PD1. Additionally, it was demonstrated that this single mutation possesses essentially equivalent binding properties compared to a more-extensively engineered PD1 variant reported in the literature. This means the test construct anti-IL-23-PD1ecd achieves favorable binding properties with a lower immunogenicity risk, since only one amino acid mutation was required. (See, FIGS. 3A-3D).

Fixed concentration of each construct was coated on the plate (1 μg/mL), followed by varying concentrations of biotinylated hu-PDL1 (detected by streptavidin-HRP). The test construct (a-IL-23-PD1 (A123I )) has a binding EC50 of 57 nM, superior to the wild type EC50 of 471 nM, but essentially equivalent to the more extensively mutated a-IL-23-PD1 (G-V2) reported in the literature. A control construct with the A123I PD1ecd mutation (but a different targeting antibody) exhibits the same binding properties as anti-IL-23-PD1 (A123I ), confirming that the Fab does not contribute to the differential binding activity observed. (See, FIG. 3A)

Varying concentrations of each construct were coated on the plate, followed by a fixed concentration of biotinylated hu-PDL1 (100 ng/ml). a-IL-23-PD1 (A123I )) has a binding EC50 of 1 μM, superior to the wild type EC50 of 3.3 μM. a-IL-23-PD1 (G-V2) is marginally better (0.6 μM). (See, FIG. 3B).

Fixed concentration of each construct was coated on the plate (1 μg/mL), followed by varying concentrations of biotinylated hu-PDL2 (detected by streptavidin-HRP). a-IL-23-PD1 (A123I )) has a binding EC50 of 54 nM, superior to the wild type EC50 of 131 nM; and essentially equivalent to anti-IL-23-PD1 (G-V2) (62 nM). (See, FIG. 3C).

Varying concentrations of each construct were coated on the plate, followed by a fixed concentration of biotinylated hu-PDL2 (500 ng/mL). a-IL-23-PD1 (A123I )) has a binding EC50 of 0.78 μM, superior to the wild type EC50 of 6 μM; and essentially equivalent to anti-IL-23-PD1 (G-V2) (0.6 μM). (See, FIG. 3D).

### Example 3: IL-23p19 Antibody Enhances the Antitumor Immune Response Induced by PDL1/PD1 Blockade

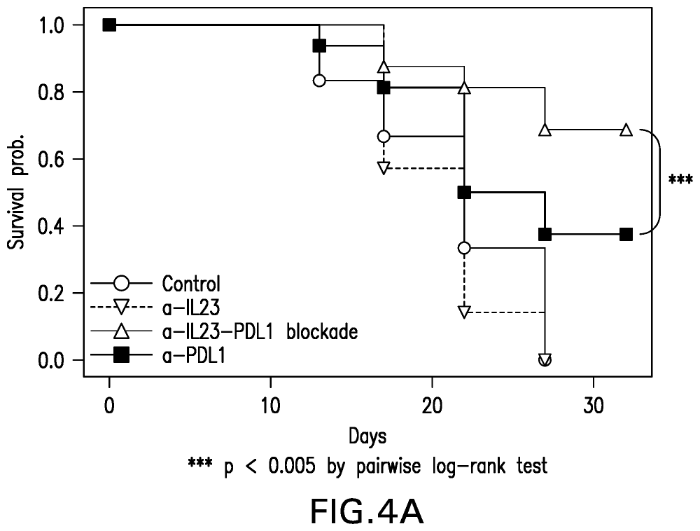

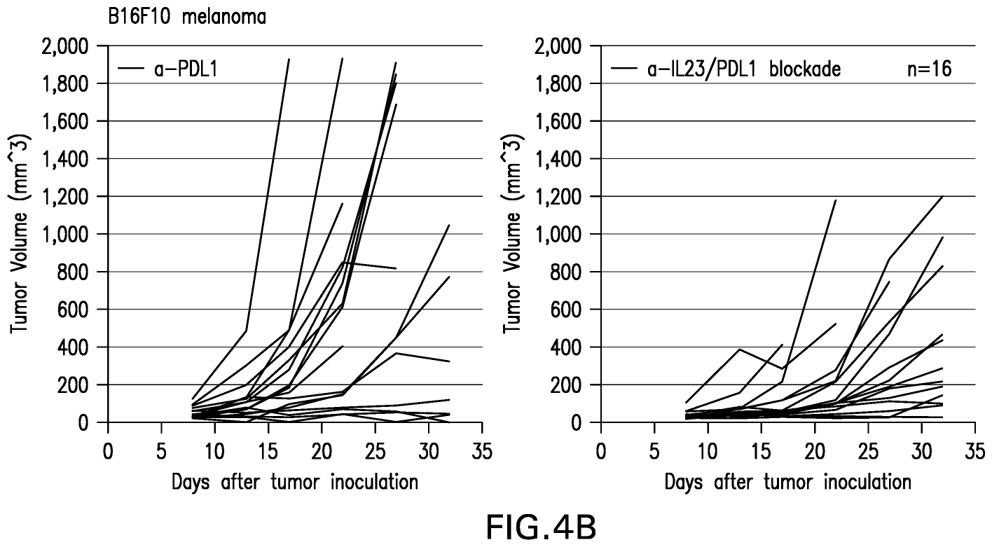

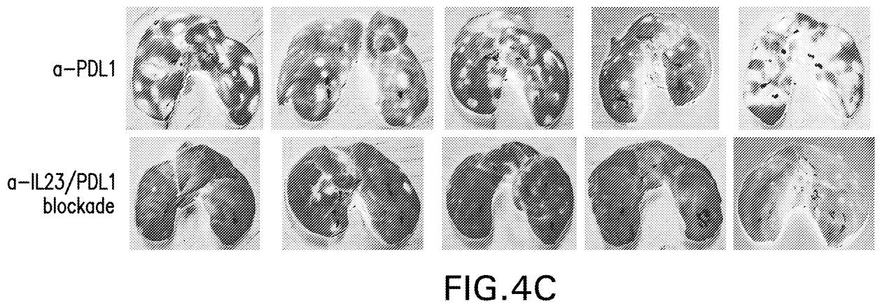

As shown in FIGS. 4A-4C, IL-23p19 antibody enhances the antitumor immune response induced by PDL1/PD1 blockade. The following tests were conducted to evaluate the hypothesis that resistance to anti-PDL1/PD1 therapy could be mediated by TH17b cells, and that such resistance could be counteracted by simultaneous blockade of PD1/PDL1 and IL-23. B16 tumor cells (4×10e5 cells, sc) were implanted in C57/BL6 mice. Once tumors reached a size of ˜50 mm³, mice were randomized and treated with either anti-PDL1 antibody (5 mg/kg i.p weekly×4) and/or anti-IL-23p19 Ab (anti-murine p19 Ab=G23-8) (5 mg/kg i.p. twice weekly×4). (See, FIGS. 4A-4C). Mice treated with the combination of a-PD-L1 Ab and a-IL-23p19 exhibited superior survival and smaller tumors compared to mice treated with a-PDL1 alone (p<0.005). (See, FIGS. 4A-4B).

The improvement in survival reflected the significant inhibition of pulmonary tumor metastases in mice treated with the combination of a-PD-L1 Ab and a-IL-23p19 compared to mice treated with a-PDL1 Ab alone. (See, FIG. 4C).

### Example 4: Anti-IL-23-PD1 Polypeptide Reduces Tumor Growth and Limits Toxicity in a Humanized Mouse Model

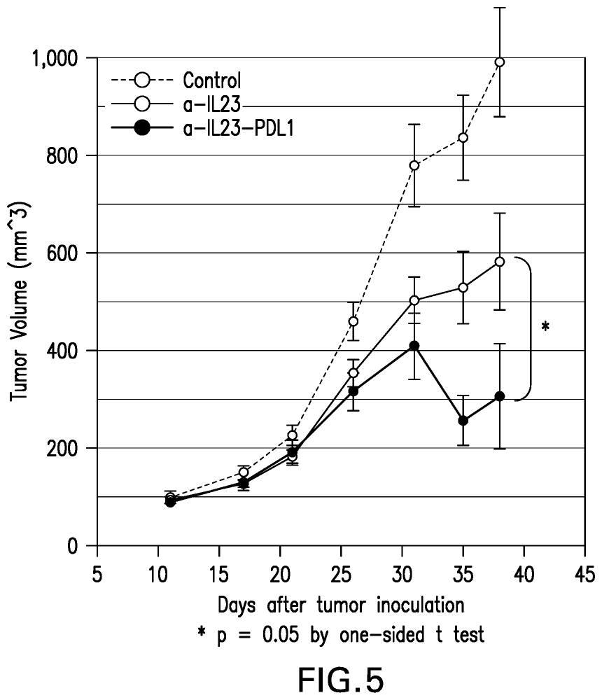

Human tumor xenografts were established in NSG mice (humanized with human PBMC). Tumor-bearing mice were randomized and treated with the following single agents or combinations: (i) Vehicle alone (control), (ii) anti-IL-23 antibody, (iii) anti-PDL1 antibody, (iv) anti-IL-23-PD1ecd. (See, FIG. 5)

In the treatment group receiving a-PDL1, 4/5 mice experienced significant GVHD during treatment. No mice in the other treatment groups did so. Anti-IL-23-PD1 resulted in significantly reduced tumor growth compared to anti-IL-23 alone. This implies that anti-IL-23-PD1 alleviated undesirable immune-related toxicity associated with anti-PDL1 treatment; while simultaneously limiting tumor growth.

### Example 5: Anti-IL-23-PD1 Polypeptide is Effective in Inhibiting Tumor Growth Compared to Treatment with Immune Checkpoint Inhibitors

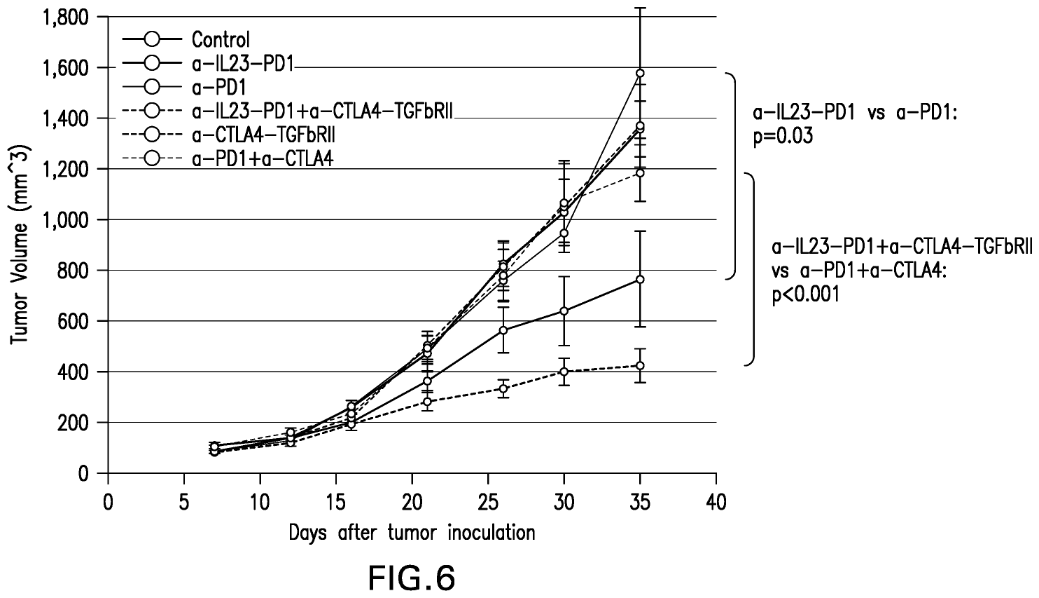

Human tumor xenografts were established in NSG mice (humanized with human PBMC). Tumor-bearing mice were randomized and treated with the following single agents or combinations: (i) Vehicle alone (control); (ii) anti-PD1 antibody (pembrolizumab); (iii) anti-PD1 antibody (pembrolizumab)+anti-CTLA4 antibody (ipilimumab); (iv) anti-IL-23-PD1; (v) anti-CTLA4-TGFbRII; (vi) anti-IL-23-PD1+CTLA4-TGFbRII (See, FIG. 6). anti-CTLA4-TGFbRII is have been reported to be more effective in reducing tumor-infiltrating Tregs and inhibiting tumor progression compared with CTLA-4 antibody (Ipilimumab) alone, as described in U.S. Pat. No. 8,993,524; Ravi et al., “Bifunctional immune checkpoint-targeted antibody-ligand traps that simultaneously disable TGFb enhance the efficacy of cancer immunotherapy.” Nat. Commun. 2018; 9:741, each of which is incorporated herein in its entirety.

Tumor-bearing mice failed to respond to treatment with either anti-PD1 (pembrolizumab) alone or even the combination of anti-PD1 and anti-CTLA4 (pembrolizumab+ipilimumab). In contrast, treatment with a-IL-23-PD1ecd alone was significantly more effective at inhibiting tumor growth compared with anti-PD1 antibody (p<0.03). Furthermore, treatment with the combination of anti-IL-23-PD1 and CTLA4-TGFbRII was able to completely arrest tumor growth, and the synergistic antitumor efficacy of this combination was strikingly superior to combined treatment with current ICI (anti-PD1+anti-CTLA4) (p<0.001). The data is consistent with the literature reports indicating a majority of cancers fail to respond to immunotherapy with antibodies targeting immune checkpoints, such as cytotoxic T-lymphocyte antigen-4 (CTLA-4) or programmed death-1 (PD-1)/PD-1 ligand (PD-L1). Ravi et al., “Bifunctional immune checkpoint-targeted antibody-ligand traps that simultaneously disable TGFb enhance the efficacy of cancer immunotherapy.” Nat. Commun. 2018; 9:741; U.S. Pat. No. 8,993,524, each of which is incorporated herein in its entirety. The synergy of IL-23 blockade with TGFb blockade demonstrates the effectiveness of combining blockade of IL-23 with blockade of an immune inhibitory cytokine.

These results demonstrate that the resistance of tumors to current ICI (either anti-PD1 alone or the combination of anti-PD1 and anti-CTLA4) can be effectively counteracted by treatment with the bifunctional fusion proteins that simultaneously disable IL-23/TGFb signaling in the tumor microenvironment (TME) (anti-IL-23-PD1 and CTLA4-TGFbRII).

### Example 6: Mechanism of Action for Anti-IL-23-PD1

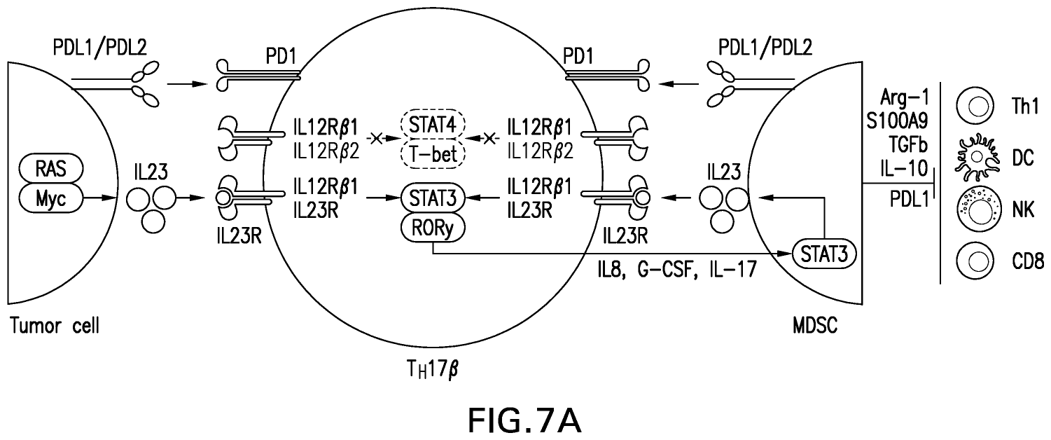

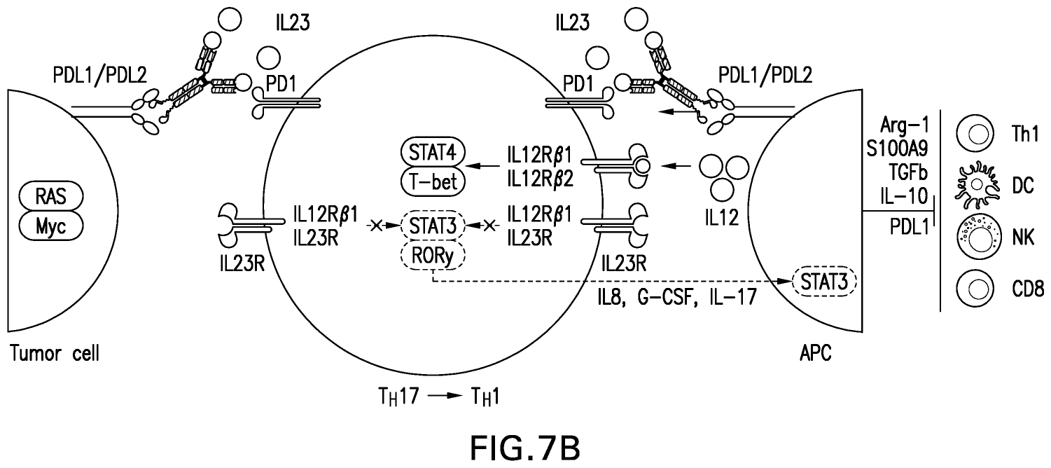

Without being bound to any theories, a possible mechanism of action for anti-IL-23-PD1 is depicted in FIGS. 7A-7B. First, the untreated tumor microenvironment (TME) is depicted in FIG. 7A. In this setting, PDL1 and PDL2 expressed on tumor cells and myeloid derived suppressor cells (MDSCs) inhibit T cell activation by binding PD1 on the T cell. In the setting of IL-23 (expressed by tumor cells and MDSCs), CD4 T cells in this TME are skewed towards a Th17 phenotype. Blockade of PD1 or PDL1/PDL2 will result in activation and proliferation of the Th17 cell. Factors derived from the Th17 cell (e.g., IL-8, G-CSF, IL-17) feed back onto the MDSC, sustaining its phenotype. Thus, in this setting, blockade of PD1 signaling alone may be counterproductive in the presence of IL-23. FIG. 7B depicts the consequences of treating with anti-IL-23-PD1: the PD1 ECD binds PDL1 and/or PDL2, neutralizing PD1 signaling. Simultaneously, PDL1+ and/or PDL2+ cells are decorated with an anti-IL-23 antibody, thereby sequestering IL-23 and counteracting the Th17 phenotype. This allows IL-12-mediated induction of an antitumoral Th1 phenotype and simultaneous activation of these Th1 cells.

SEQUENCE LISTING

| NO. | NAME | SEQUENCE |
|---|---|---|
| 1 | IL-23R-ECD | GITNINCSGHIWVEPATIFKMGMNISIYCQAAIKNCQPRKLHFYKNGIKERFQIT |
| | (Q5VWK5\| | RINKTTARLWYKNFLEPHASMYCTAECPKHFQETLICGKDISSGYPPDIPDEVT |
| | 24-355) | CVIYEYSGNMTCTWNAGKLTYIDTKYVVHVKSLETEEEQQYLTSSYINISTDSL |
| | | QGGKKYLVWVQAANALGMEESKQLQIHLDDIVIPSAAVISRAETINATVPKTII |
| | | YWDSQTTIEKVSCEMRYKATTNQTWNVKEFDTNFTYVQQSEFYLEPNIKYVF |
| | | QVRCQETGKRYWQPWSSLFFHKTPETVPQVTSKAFQHDTWNSGLTVASISTG |
| | | HLTSDNRGDIG |
| 2 | IL-23R-ECD | CSGHIWVEPATIFKMGMNISIYCQAAIKNCQPRKLHFYKNGIKERFQITRINKTT |
| | (D1) | ARLWYKNFLEPHASMYCTAECPKHFQETLICGKDISSGYPP |
| 3 | IL-23R-ECD | DIPDEVTCVIYEYSGNMTCTWNAGKLTYIDTKYVVHVKSLETEEEQQYLTSSYI |
| | (D2) | NISTDSLQGGKKYLVWVQAANALGMEESKQLQIHLD |
| 4 | IL-23R-ECD | DIVIPSAAVISRAETINATVPKTIIYWDSQTTIEKVSCEMRYKATTNQTWNVKEF |
| | (D3) | DTNFTYVQQSEFYLEPNIKYVFQVRCQETGKRYWQPWSSLFFHKTPET |
| 5 | IL-23R-ECD | GITNINCSGHIWVEPATIFKMGMNISIYCQAAIKNCQPRKLHFYKNGIKERFQIT |
| | (DID2) | RINKTTARLWYKNFLEPHASMYCTAECPKHFQETLICGKDISSGYPPDIPDEVT |
| | | CVIYEYSGNMTCTWNAGKLTYIDTKYVVHVKSLETEEEQQYLTSSYINISTDSL |
| | | QGGKKYLVWVQAANALGMEESKQLQIHLD |
| 6 | IL-23R-ECD | GITNINCSGHIWVEPATIFKMGMNISIYCQAAIKNCQPRKLHFYKNGIKERFQIT |
| | (DID2D3) | RINKTTARLWYKNFLEPHASMYCTAECPKHFQETLICGKDISSGYPPDIPDEVT |
| | | CVIYEYSGNMTCTWNAGKLTYIDTKYVVHVKSLETEEEQQYLTSSYINISTDSL |
| | | QGGKKYLVWVQAANALGMEESKQLQIHLD |
| | | DIVIPSAAVISRAETINATVPKTIIYWDSQTTIEKVSCEMRYKATTNQTWNVKEF |
| | | DTNFTYVQQSEFYLEPNIKYVFQVRCQETGKRYWQPWSSLFFHKTPET |
| 7 | IL 12Rb-ECD | CRTSECCFQDPPYPDADSGSASGPRDLRCYRISSDRYECSWQYEGPTAGVSHFL |
| | (P42701\| | RCCLSSGRCCYFAAGSATRLQFSDQAGVSVLYTVTLWVESWARNQTEKSPEV |
| | 24-545) | TLQLYNSVKYEPPLGDIKVSKLAGQLRMEWETPDNQVGAEVQFRHRTPSSPW |
| | | KLGDCGPQDDDTESCLCPLEMNVAQEFQLRRRQLGSQGSSWSKWSSPVCVPP |
| | | ENPPQPQVRFSVEQLGQDGRRRLTLKEQPTQLELPEGCQGLAPGTEVTYRLQL |
| | | HMLSCPCKAKATRTLHLGKMPYLSGAAYNVAVISSNQFGPGLNQTWHIPADT |
| | | HTEPVALNISVGTNGTTMYWPARAQSMTYCIEWQPVGQDGGLATCSLTAPQD |
| | | PDPAGMATYSWSRESGAMGQEKCYYITIFASAHPEKLTLWSTVLSTYHFGGNA |
| | | SAAGTPHHVSVKNHSLDSVSVDWAPSLLSTCPGVLKEYVVRCRDEDSKQVSE |
| | | HPVQPTETQVTLSGLRAGVAYTVQVRADTAWLRGVWSQPQRFSIEVQVSD |
| 8 | IL 12Rb (D1) | GPRDLRCYRISSDRYECSWQYEGPTAGVSHFLRCCLSSGRCCYFAAGSATRLQ |
| | (P42701\| | FSDQAGVSVLYTVTLWVESWARNQTEKSPEVTLQLYNS |
| | 46-136) | |
| 9 | IL 12Rb (D2) | PLGDIKVSKLAGQLRMEWETPDNQVGAEVQFRHRTPSSPWKLGDCGPQDDDT |
| | (P42701\| | ESCLCPLEMNVAQEFQLRRRQLGSQGSSWSKWSSPVCVPPE |
| | 142-234) | |
| 10 | IL 12Rb | GPRDLRCYRISSDRYECSWQYEGPTAGVSHFLRCCLSSGRCCYFAAGSATRLQ |
| | (DID2) | FSDQAGVSVLYTVTLWVESWARNQTEKSPEVTLQLYNSPLGDIKVSKLAGQL |
| | | RMEWETPDNQVGAEVQFRHRTPSSPWKLGDCGPQDDDTESCLCPLEMNVAQ |
| | | EFQLRRRQLGSQGSSWSKWSSPVCVPPE |
| 11 | Anti-CEA | EVOLVESGGGVVQPGRSLRLSCSASGFDFTTYWMSWVRQAPGKGLEWIGEIH |
| | antibody | PDSSTINYAPSLKDRFTISRDNAKNTLFLQMDSLRPEDTGVYFCASLYFGFPWF |
| | (labetuzumab)- | AYWGQGTPVTVSSASTKGPSVFPLAPSSKSTSGGTAALGCLVKDYFPEPVTVS |
| | HC | WNSGALTSGVHTFPAVLQSSGLYSLSSVVTVPSSSLGTQTYICNVNHKPSNTKV |
| | | DKRVEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQDWLNG |
| | | KEYKCKVSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRWQQGNVF |
| | | SCSVMHEALHNHYTQKSLSLSPGK |
| 12 | Anti-CEA | DIQLTQSPSSLSASVGDRVTITCKASQDVGTSVAWYQQKPGKAPKLLIYWTST |
| | antibody | RHTGVPSRFSGSGSGTDFTFTISSLQPEDIATYYCQQYSLYRSFGQGTKVEIKRT |
| | (labetuzumab)- | VAAPSVFIFPPSDEQLKSGTASVVCLLNNFYPREAKVQWKVDNALQSGNSQES |
| | LC | VTEQDSKDSTYSLSSTLTLSKADYEKHKVYACEVTHQGLSSPVTKSFNRGEC |
| 13 | anti-CEA | SEQ ID NO: 11 + SEQ ID NO: 55 + SEQ ID NO: 2 |
| | antibody with | |
| | IL-23RD1 | |
| | fused to C | |
| | terminus of | |
| | HC | |
| 14 | anti-CEA | SEQ ID NO: 12 + SEQ ID NO: 55 + SEQ ID NO: 2 |
| | antibody with | |
| | IL-23RD1 | |
| | fused to C | |
| | terminus of LC | |
| 15 | anti-CEA | SEQ ID NO: 11 + SEQ ID NO: 55 + SEQ ID NO: 5 |
| | antibody with | |
| | IL-23RD1D2 | |
| | fused to C | |
| | terminus of | |
| | HC | |
| 16 | anti-CEA | SEQ ID NO: 12 + SEQ ID NO: 55 + SEQ ID NO: 5 |
| | antibody with | |
| | IL-23RD1D2 | |
| | fused to C | |
| | terminus of LC | |
| 17 | anti-CEA | SEQ ID NO: 11 + SEQ ID NO: 55 + SEQ ID NO: 6 |
| | antibody with | |
| | IL- | |
| | 23RD1D2D3 | |
| | fused to C | |
| | terminus of | |
| | HC | |
| 18 | anti-CEA | SEQ ID NO: 12 + SEQ ID NO: 55 + SEQ ID NO: 2 |
| | antibody with | |
| | IL- | |
| | 23RD1D2D3 | |
| | fused to C | |
| | terminus of LC | |
| 19 | anti-CEA | SEQ ID NO: 11 + SEQ ID NO: 55 + SEQ ID NO: 58 |
| | antibody with | |
| | TGFbRII-ECD | |
| | fused to C | |
| | terminus of | |
| | HC | |
| 20 | TNFR-ECD- | SEQ ID NO: 75 + SEQ ID NO: 114 + SEQ ID NO: 55 + SEQ ID NO: 2 |
| | Fc-IL-23RD1 | |
| 21 | TNFR-ECD- | SEQ ID NO: 75 + SEQ ID NO: 114 + SEQ ID NO: 55 + SEQ ID NO: 5 |
| | Fc-IL- | |
| | 23RD1D2 | |
| 22 | TNFR-ECD- | SEQ ID NO: 75 + SEQ ID NO: 114 + SEQ ID NO: 55 + SEQ ID NO: 6 |
| | Fc-IL- | |
| | 23RD1D2D3 | |
| 23 | Fc-IL-23RD1 | SEQ ID NO: 114 + SEQ ID NO: 55 + SEQ ID NO: 2 |
| 24 | Fc-IL- | SEQ ID NO: 114 + SEQ ID NO: 55 + SEQ ID NO: 5 |
| | 23RD1D2 | |
| 25 | Fc-IL- | SEQ ID NO: 114 + SEQ ID NO: 55 + SEQ ID NO: 6 |
| | 23RD1D2D3 | |
| 26 | IL-23RD1-Fc | SEQ ID NO: 2 + SEQ ID NO: 114 |
| 27 | IL-23RD1D2- | SEQ ID NO: 5 + SEQ ID NO: 114 |
| | Fc | |
| 28 | IL- | SEQ ID NO: 6 + SEQ ID NO: 114 |
| | 23RD1D2D3- | |
| | Fc | |
| 29 | IL-23R/IL 12R- | SEQ ID NO: 8 + SEQ ID NO: 55 + SEQ ID NO: 2 |
| | ECD | |
| | IL 12RbD1- | |
| | linker-IL- | |
| | 23RD1 | |
| 30 | IL-23R/IL 12R- | SEQ ID NO: 8 + SEQ ID NO: 55 + SEQ ID NO: 5 |
| | ECD | |
| | IL 12RbD1- | |
| | linker-IL- | |
| | 23RD1D2 | |
| 31 | IL-23R/IL 12R- | SEQ ID NO: 8 + SEQ ID NO: 55 + SEQ ID NO: 6 |
| | ECD: | |
| | IL12RbD1- | |
| | linker-IL- | |
| | 23RD1D2D3 | |
| 32 | IL-23R/IL12R- | SEQ ID NO: 10 + SEQ ID NO: 55 + SEQ ID NO: 2 |
| | ECD: | |
| | IL 12RbDID2- | |
| | linker-IL- | |
| | 23RD1 | |
| 33 | IL-23R/IL 12R- | SEQ ID NO: 10 + SEQ ID NO: 55 + SEQ ID NO: 5 |
| | ECD | |
| | IL 12RbDID2- | |
| | linker-IL- | |
| | 23RD1D2 | |
| 34 | IL-23R/IL 12R- | SEQ ID NO: 2 + SEQ ID NO: 55 + SEQ ID NO: 8 |
| | ECD: IL- | |
| | 23RD1-linker- | |
| | IL12RbD1 | |
| 35 | IL-23R/IL 12R- | SEQ ID NO: 5 + SEQ ID NO: 55 + SEQ ID NO: 8 |
| | ECD: IL- | |
| | 23RD1D2- | |
| | linker- | |
| | IL12RbD1 | |
| 36 | IL-23R/IL 12R- | SEQ ID NO: 6 + SEQ ID NO: 55 + SEQ ID NO: 8 |
| | ECD: IL- | |
| | 23RD1D2D3- | |
| | linker- | |
| | IL12RbD1 | |
| 37 | IL-23R/IL 12R- | SEQ ID NO: 2 + SEQ ID NO: 55 + SEQ ID NO: 10 |
| | ECD: IL- | |
| | 23RD1-linker- | |
| | IL 12RbDID2 | |
| 38 | IL-23R/IL 12R- | SEQ ID NO: 5 + SEQ ID NO: 55 + SEQ ID NO: 10 |
| | ECD: IL- | |
| | 23RD1D2- | |
| | linker- | |
| | IL 12RbDID2 | |
| 39 | anti-CEA with | SEQ ID NO: 11 + SEQ ID NO: 55 + SEQ ID NO: 34 |
| | IL 12RbD1- | |
| | linker-IL- | |
| | 23RD 1 fused | |
| | to C terminus | |
| | of HC | |
| 40 | anti-CEA with | SEQ ID NO: 12 + SEQ ID NO: 55 + SEQ ID NO: 34 |
| | IL 12RbD1- | |
| | linker-IL- | |
| | 23RD 1 fused | |
| | to C terminus | |
| | of LC | |
| 41 | VEGFR-Fc- | SEQ ID NO: 61 + SEQ ID NO: 114 + SEQ ID NO: 55 + SEQ ID NO: 2 |
| | IL-23RD1 | |
| 42 | IL-23RD1-Fc- | SEQ ID NO: 2 + SEQ ID NO: 114 + SEQ ID NO: 61 |
| | VEGFR | |
| 43 | TGFbRII-Fc- | SEQ ID NO: 58 + SEQ ID NO: 114 + SEQ ID NO: 55 + SEQ ID NO: 2 |
| | IL-23RD1 | |
| 44 | IL-23RD1-Fc- | SEQ ID NO: 2 + SEQ ID NO: 114 + SEQ ID NO: 55 + SEQ ID NO: 58 |
| | TGFbRII | |
| 45 | PSMA-binding | WQPDTAHHWATLDKTHTCPPCPAPELLGGPSVFLFPPKPKDTLMISRTPEVTCV |
| | peptide fused | VVDVSHEDPEVKFNWYVDGVEVHNAKTKPREEQYNSTYRVVSVLTVLHQDW |
| | to Fc and IL- | LNGKEYKCKVSNKALPAPIEKTISKAKGQPREPQVYTLPPSRDELTKNQVSLTC |
| | 23RD1 | LVKGFYPSDIAVEWESNGQPENNYKTTPPVLDSDGSFFLYSKLTVDKSRWQQG |
| | | NVFSCSVMHEALHNHYTQKSLSLSPGGGGGSGGGGSGGGGSGITNINCSGHIW |
| | | VEPATIFKMGMNISIYCQAAIKNCQPRKLHFYKNGIKERFQITRINKTTARLWY |
| | | KNFLEPHASMYCTAECPKHFQETLICGKDISSGYPP |
| 46 | Anti-PSMA | QVQLVESGGGVVQPGRSLRLSCAASGFAFSRYGMHWVRQAPGKGLEWVAVI |
| | antibody heavy | WYDGSNKYYADSVKGRFTISRDNSKNTQYLQMNSLRAEDTAVYYCARGGDF |
| | chain (from | LYYYYYGMDVWGQGTTVTVSSASTKGPSVFPLAPSSKSTSGGTAALGCL VKD |
| | patent | YFPEPVTVSWNSGALTSGVHTFPAVLQSSGLYSLSSVVTVPSSSLGTQTYICNV |
| | US8114965- | NHKPSNTKVDKKVEPKSCDKTHTCPPCPAPELLGGPSVFLFPPKPKDTLMISRT |
| | SEQ ID NO: 15) | PEVTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLYSKLTVD |
| | | KSRWQQGNVFSCSVMHEALHNHYTQKSLSLSPGK |
| 47 | Anti-PSMA | DIQMTQSPSSLSASVGDRVTITCRASQGISNYLAWYQQKTGKVPKFLIYEASTL |
| | antibody light | QSGVPSRFSGGGSGTDFTLTISSLQPEDVATYYCQNYNSAPFTFGPGTKVDIKR |
| | chain (from | TVAAPSVFIFPPSDEQLKSGTASVVCLLNNFYPREAKVQWKVDNALQSGNSQE |
| | patent | SVTEQDSKDSTYSLSSTLTLSKADYEKHKVYACEVTHQGLSSPVTKSFNRGEC |
| | US8114965- | |
| | SEQ ID NO: 17) | |
| 48 | anti-PSMA | SEQ ID NO: 46 + SEQ ID NO: 55 + SEQ ID NO: 2 |
| | antibody with | |
| | IL-23RD1 | |
| | fused to C | |
| | terminus of | |
| | HC | |
| 49 | anti-PSMA | SEQ ID NO: 47 + SEQ ID NO: 55 + SEQ ID NO: 2 |
| | antibody with | |
| | IL-23RD1 | |
| | fused to C | |
| | terminus of LC | |
| 50 | anti-PSMA | SEQ ID NO: 46 + SEQ ID NO: 55 + SEQ ID NO: 58 |
| | antibody with | |
| | TGFbRII-ECD | |
| | fused to C | |
| | terminus of | |
| | HC | |
| 51 | anti-IL-23 | SEQ ID NO: 47 + SEQ ID NO: 55 + SEQ ID NO: 58 |
| | antibody with | |
| | TGFbRII-ECD | |
| | fused to C | |
| | terminus of | |
| | HC | |
| 52 | anti-IL-23 | DIQMTQSPSSLSASVGDRVTITCKASRDVAIAVAWYQQKPGKVPKLLIYWAST |
| | antibody with | RHTGVPSRFSGSGSRTDFTLTISSLQPEDVADYFCHQYSSYPFTFGSGTKLEIKR |
| | PSMA-binding | TVAAPSVFIFPPSDEQLKSGTASVVCLLNNFYPREAKVQWKVDNALQSGNSQE |
| | peptide fused | SVTEQDSKDSTYSLSSTLTLSKADYEKHKVYACEVTHQGLSSPVTKSFNRGEC |
| | to C terminus | GGGGSGGGGSGGGGSWQPDTAHHWATL |
| | of LC | |
| 53 | Anti-IL-23- | EVQLVQSGAEVKKPGESLKISCKGSGYSFSNYWIGWVRQMPGKGLEWMGIID |
| | PD1-HC; | PSNSYTRYSPSFQGQVTISADKSISTAYLQWSSLKASDTAMYYCARWYYKPFD |
| | guselkumab | VWGQGTLVTVSSASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPSNTKVD |
| | | KKVEPKSCDKTHTCPPCPAPEAAGGPSVFLFPPKPKDTLMISRTPEVTCVVVDV |
| | | SHEDPEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLTCLVKG |
| | | FYPSDIAVEWESNGQPENNYKTTPPVLDSDGSFFLYSKLTVDKSRWQQGNVFS |
| | | CSVMHEALHNHYTQKSLSLSPGKGGGGSGGGGSGGGGSPGWFLDSPDRPWNP |
| | | PTFSPALLVVTEGDNATFTCSFSNTSESFVLNWYRMSPSNQTDKLAAFPEDRSQ |
| | | PGQDCRFRVTQLPNGRDFHMSVVRARRNDSGTYLCGAISLAPKIQIKESLRAEL |
| | | RVTERRAEVPTAHPSPSPRPAGQFQTLV |
| 54 | Anti-IL-23- | QSVLTQPPSVSGAPGQRVTISCTGSSSNIGSGYDVHWYQQLPGTAPKLLIYGNS |
| | LC; | KRPSGVPDRFSGSKSGTSASLAITGLQSEDEADYYCASWTDGLSLVVFGGGTK |
| | guselkumab | LTVLGQPKAAPSVTLFPPSSEELQANKATLVCLISDFYPGAVTVAWKADSSPVK |
| | | AGVETTTPSKQSNNKYAASSYLSLTPEQWKSHRSYSCQVTHEGSTVEKTVAPT |
| | | ECS |
| 55 | (GGGGS)3 | GGGGSGGGGSGGGGS |
| | linker | |
| 56 | Human PD1 | PGWFLDSPDRPWNPPTFSPALLVVTEGDNATFTCSFSNTSESFVLNWYRMSPSN |
| | ECD (WT) | QTDKLAAFPEDRSQPGQDCRFRVTQLPNGRDFHMSVVRARRNDSGTYLCGAIS |
| | | LAPKAQIKESLRAELRVTERRAEVPTAHPSPSPRPAGQFQTLV |
| 57 | Human PD1 | PGWFLDSPDRPWNPPTFSPALLVVTEGDNATFTCSFSNTSESFVLNWYRMSPSN |
| | ECD-A132I | QTDKLAAFPEDRSQPGQDCRFRVTQLPNGRDFHMSVVRARRNDSGTYLCGAIS |
| | | LAPKIQIKESLRAELRVTERRAEVPTAHPSPSPRPAGQFQTLV |
| 58 | TGFbRII ECD | TIPPHVQKSVNNDMIVTDNNGAVKFPQLCKFCDVRFSTCDNQKSCMSNCSITSI |
| | | CEKPQEVCVAVWRKNDENITLETVCHDPKLPYHDFILEDAASPKCIMKEKKKP |
| | | GETFFMCSCSSDECNDNIIFSEEYNTSNPD |
| 59 | VEGFR1 ECD | SKLKDPELSLKGTQHIMQAGQTLHLQCRGEAAHKWSLPEMVSKESERLSITKS |
| | | ACGRNGKQFCSTLTLNTAQANHTGFYSCKYLAVPTSKKKETESAIYIFISDTGR |
| | | PFVEMYSEIPEIIHMTEGRELVIPCRVTSPNITVTLKKFPLDTLIPDGKRIIWDSRK |
| | | GFIISNATYKEIGLLTCEATVNGHLYKTNYLTHRQTNTIIDVQISTPRPVKLLRG |
| | | HTLVLNCTATTPLNTRVQMTWSYPDEKNKRASVRRRIDQSNSHANIFYSVLTI |
| | | DKMQNKDKGLYTCRVRSGPSFKSVNTSVHIYDKAFITVKHRKQQVLETVAGK |
| | | RSYRLSMKVKAFPSPEVVWLKDGLPATEKSARYLTRGYSLIIKDVTEEDAGNY |
| | | TILLSIKQSNVFKNLTATLIVNVKPQIYEKAVSSFPDPALYPLGSRQILTCTAYGI |
| | | PQPTIKWFWHPCNHNHSEARCDFCSNNEESFILDADSNMGNRIESITQRMAIIEG |
| | | KNKMASTLVVADSRISGIYICIASNKVGTVGRNISFYITDVPNGFHVNLEKMPT |
| | | EGEDLKLSCTVNKFLYRDVTWILLRTVNNRTMHYSISKQKMAITKEHSITLNLT |
| | | IMNVSLQDSGTYACRARNVYTGEEILQKKEITIRDQEAPYLLRNLSDHTVAISSS |
| | | TTLDCHANGVPEPQITWFKNNHKIQQEPGIILGPGSSTLFIERVTEEDEGVYHCK |
| | | ATNQKGSVESSAYLTVQGTSDKSNLE |
| 60 | VEGFR2 ECD | ASVGLPSVSLDLPRLSIQKDILTIKANTTLQITCRGQRDLDWLWPNNQSGSEQR |
| | | VEVTECSDGLFCKTLTIPKVIGNDTGAYKCFYRETDLASVIYVYVQDYRSPFIA |
| | | SVSDQHGVVYITENKNKTVVIPCLGSISNLNVSLCARYPEKRFVPDGNRISWDS |
| | | KKGFTIPSYMISYAGMVFCEAKINDESYQSIMYIVVVVGYRIYDVVLSPSHGIEL |
| | | SVGEKLVLNCTARTELNVGIDFNWEYPSSKHQHKKLVNRDLKTQSGSEMKKF |
| | | LSTLTIDGVTRSDQGLYTCAASSGLMTKKNSTFVRVHEKPFVAFGSGMESLVE |
| | | ATVGERVRIPAKYLGYPPPEIKWYKNGIPLESNHTIKAGHVLTIMEVSERDTGN |
| | | YTVILTNPISKEKQSHVVSLVVYVPPQIGEKSLISPVDSYQYGTTQTLTCTVYAI |
| | | PPPHHIHWYWQLEEECANEPSQAVSVTNPYPCEEWRSVEDFQGGNKIEVNKN |
| | | QFALIEGKNKTVSTLVIQAANVSALYKCEAVNKVGRGERVISFHVTRGPEITLQ |
| | | PDMQPTEQESVSLWCTADRSTFENLTWYKLGPQPLPIHVGELPTPVCKNLDTL |
| | | WKLNATMFSNSTNDILIMELKNASLQDQGDYVCLAQDRKTKKRHCVVRQLT |
| | | VLERVAPTITGNLENQTTSIGESIEVSCTASGNPPPQIMWFKDNETLVEDSGIVL |
| | | KDGNRNLTIRRVRKEDEGLYTCQACSVLGCAKVEAFFIIEGAQEKTNLE |
| 61 | VEGFR1 D2 + | SDTGRPFVEMYSEIPEIIHMTEGRELVIPCRVTSPNITVTLKKFPLDTLIPDGKRII |
| | VEGFR2 D3 | WDSRKGFIISNATYKEIGLLTCEATVNGHLYKTNYLTHRQTNTIIDVVLSPSHGI |
| | | ELSVGEKLVLNCTARTELNVGIDFNWEYPSSKHQHKKLVNRDLKTQSGSEMK |
| | | KFLSTLTIDGVTRSDQGLYTCAASSGLMTKKNSTFVRVHEK |
| 62 | BTLA ECD | KESCDVQLYIKRQSEHSILAGDPFELECPVKYCANRPHVTWCKLNGTTCVKLE |
| | | DRQTSWKEEKNISFFILHFEPVLPNDNGSYRCSANFQSNLIESHSTTLYVTDVKS |
| | | ASERPSKDEMAS |
| 63 | TIM3 ECD | SEVEYRAEVGQNAYLPCFYTPAAPGNLVPVCWGKGACPVFECGNVVLRTDER |
| | | DVNYWTSRYWLNGDFRKGDVSLTIENVTLADSGIYCCRIQIPGIMNDEKFNLK |
| | | LVIKPAKVTPAPTRQRDFTAAFPRMLTTRGHGPAETQTLGSLPDINLTQISTLAN |
| | | ELRDSRLANDLRDSGATIRIG |
| 64 | IL 1OR ECD | HGTELPSPPSVWFEAEFFHHILHWTPIPNQSESTCYEVALLRYGIESWNSISNCS |
| | | QTLSYDLTAVTLDLYHSNGYRARVRAVDGSRHSNWTVTNTRFSVDEVTLTVG |
| | | SVNLEIHNGFILGKIQLPRPKMAPANDTYESIFSHFREYEIAIRKVPGNFTFTHKK |
| | | VKHENFSLLTSGEVGEFCVQVKPSVASRSNKGMWSKEECISLTRQYFTVTN |
| 65 | LIGHT ECD | DGPAGSWEQLIQERRSHEVNPAAHLTGANSSLTGSGGPLLWETQLGLAFLRGL |
| | | SYHDGALVVTKAGYYYIYSKVQLGGVGCPLGLASTITHGLYKRTPRYPEELEL |
| | | LVSQQSPCGRATSSSRVWWDSSFLGGVVHLEAGEKVVVRVLDERLVRLRDGT |
| | | RSYFGAFMV |
| 66 | CD80 ECD | VIHVTKEVKEVATLSCGHNVSVEELAQTRIYWQKEKKMVLTMMSGDMNIWP |
| | | EYKNRTIFDITNNLSIVILALRPSDEGTYECVVLKYEKDAFKREHLAEVTLSVK |
| | | ADFPTPSISDFEIPTSNIRRIICSTSGGFPEPHLSWLENGEELNAINTTVSQDPETEL |
| | | YAVSSKLDFNMTTNHSFMCLIKYGHLRVNQTFNWNTTKQEHFPDN |
| 67 | CD86 ECD | APLKIQAYFNETADLPCQFANSQNQSLSELVVFWQDQENLVLNEVYLGKEKFD |
| | | SVHSKYMGRTSFDSDSWTLRLHNLQIKDKGLYQCIIHHKKPTGMIRIHQMNSE |
| | | LSVLANFSQPEIVPISNITENVYINLTCSSIHGYPEPKKMSVLLRTKNSTIEYDGV |
| | | MQKSQDNVTELYDVSISLSVSFPDVTSNMTIFCILETDKTRLLSSPFSIELEDPQP |
| | | PPDHIP |
| 68 | SIRPa ECD | EEELQVIQPDKSVLVAAGETATLRCTATSLIPVGPIQWFRGAGPGRELIYNQKE |
| | | GHFPRVTTVSDLTKRNNMDFSIRIGNITPADAGTYYCVKFRKGSPDDVEFKSGA |
| | | GTELSVRAKPS |
| 69 | CD30L ECD | QRTDSIPNSPDNVPLKGGNCSEDLLCILKRAPFKKSWAYLQVAKHLNKTKLSW |
| | | NKDGILHGVRYQDGNLVIQFPGLYFIICQLQFLVQCPNNSVDLKLELLINKHIKK |
| | | QALVTVCESGMQTKHVYQNLSQFLLDYLQVNTTISVNVDTFQYIDTSTFPLEN |
| | | VLSIFLYSNSD |
| 70 | 41BBL ECD | ACPWAVSGARASPGSAASPRLREGPELSPDDPAGLLDLRQGMFAQLVAQNVL |
| | | LIDGPLSWYSDPGLAGVSLTGGLSYKEDTKELVVAKAGVYYVFFQLELRRVV |
| | | AGEGSGSVSLALHLQPLRSAAGAAALALTVDLPPASSEARNSAFGFQGRLLHL |
| | | SAGQRLGVHLHTEARARHAWQLTQGATVLGLFRVTPEIPAGLPSPRSE |
| 71 | OX40L ECD | QVSHRYPRIQSIKVQFTEYKKEKGFILTSQKEDEIMKVQNNSVIINCDGFYLISL |
| | | KGYFSQEVNISLHYQKDEEPLFQLKKVRSVNSLMVASLTYKDKVYLNVTTDN |
| | | TSLDDFHVNGGELILIHQNPGEFCVL |
| 72 | GITRL ECD | QLETAKEPCMAKFGPLPSKWQMASSEPPCVNKVSDWKLEILQNGLYLIYGQV |
| | | APNANYNDVAPFEVRLYKNKDMIQTLTNKSKIQNVGGTYELHVGDTIDLIENS |
| | | EHQVLKNNTYWGIILLANPQFIS |
| 73 | ICOSL ECD | DTQEKEVRAMVGSDVELSCACPEGSRFDLNDVYVYWQTSESKTVVTYHIPQN |
| | | SSLENVDSRYRNRALMSPAGMLRGDFSLRLFNVTPQDEQKFHCLVLSQSLGFQ |
| | | EVLSVEVTLHVAANFSVPVVSAPHSPSQDELTFTCTSINGYPRPNVYWINKTDN |
| | | SLLDQALQNDTVFLNMRGLYDVVSVLRIARTPSVNIGCCIENVLLQQNLTVGS |
| | | QTGNDIGERDKITENPVSTGEKNAATWS |
| 74 | CD40L ECD | GDQNPQIAAHVISEASSKTTSVLQWAEKGYYTMSNNLVTLENGKQLTVKRQG |
| | | LYYIYAQVTFCSNREASSQAPFIASLCLKSPGRFERILLRAANTHSSAKPCGQQS |
| | | IHLGGVFELQPGASVFVNVTDPSQVSHGTGFTSFGLLKL |
| 75 | TNFR2 ECD | LPAQVAFTPYAPEPGSTCRLREYYDQTAQMCCSKCSPGQHAKVFCTKTSDTVC |
| | | DSCEDSTYTQLWNWVPECLSCGSRCSSDQVETQACTREQNRICTCRPGWYCA |
| | | LSKQEGCRLCAPLRKCRPGFGVARPGTETSDVVCKPCAPGTFSNTTSSTDICRP |
| | | HQICNVVAIPGNASMDAVCTSTSPTRSMAPGAVHLPQPVSTRSQHTQPTPEPST |
| | | APSTSFLLPMGPSPPAEGSTGD |
| 76 | RANK ECD | IAPPCTSEKHYEHLGRCCNKCEPGKYMSSKCTTTSDSVCLPCGPDEYLDSWNE |
| | | EDKCLLHKVCDTGKALVAVVAGNSTTPRRCACTAGYHWSQDCECCRRNTEC |
| | | APGLGAQHPLQLNKDTVCKPCLAGYFSDAFSSTDKCRPWTNCTFLGKRVEHH |
| | | GTEKSDAVCSSSLPARKPPNEPHVYLP |
| 77 | IL IRA | MEICRGLRSHLITLLLFLFHSETICRPSGRKSSKMQAFRIWDVNQKTFYLRNNQ |
| | | LVAGYLQGPNVNLEEKIDVVPIEPHALFLGIHGGKMCLSCVKSGDETRLQLEA |
| | | VNITDLSENRKQDKRFAFIRSDSGPTTSFESAACPGWFLCTAMEADQPVSLTNM |
| | | PDEGVMVTKFYFQEDE |
| 78 | IL22BP ECD | MMPKHCFLGFLISFFLTGVAGTQSTHESLKPQRVQFQSRNFHNILQWQPGRAL |
| | | TGNSSVYFVQYKIMFSCSMKSSHQKPSGCWQHISCNFPGCRTLAKYGQRQWK |
| | | NKEDCWGTQELSCDLTSETSDIQEPYYGRVRAASAGSYSEWSMTPRFTPWWE |
| | | TKIDPPVMNITQVNGSLLVILHAPNLPYRYQKEKNVSIEDYYELLYRVFIINNSL |
| | | EKEQKVYEGAHRAVEIEALTPHSSYCVVAEIYQPMLDRRSQRSEERCVEIP |
| 79 | risankizumab | QVQLVQSGAEVKKPGSSVKVSCKASGYTFTDQTIHWMRQAPGQGLEWIGYIY |
| | VH | PRDDSPKYNENFKGKVTITADKSTSTAYMELSSLRSEDTAVYYCAIPDRSGYA |
| | | WFIYWGQGTLVTVSS |
| 80 | risankizumab- | DIQMTQSPSSLSASVGDRVTITCKASRDVAIAVAWYQQKPGKVPKLLIYWAST |
| | VL | RHTGVPSRFSGSGSRTDFTLTISSLQPEDVADYFCHQYSSYPFTFGSGTKLEIK |
| 81 | guselkumab- | EVQLVQSGAEVKKPGESLKISCKGSGYSFSNYWIGWVRQMPGKGLEWMGIID |
| | VH | PSNSYTRYSPSFQGQVTISADKSISTAYLQWSSLKASDTAMYYCARWYYKPFD |
| | | VWGQGTLVTVSS |
| 82 | guselkumab- | QSVLTQPPSVSGAPGQRVTISCTGSSSNIGSGYDVHWYQQLPGTAPKLLIYGNS |
| | VL | KRPSGVPDRFSGSKSGTSASLAITGLQSEDEADYYCASWTDGLSLVVFGGGTK |
| | | LTVL |
| 83 | tildrakizumab- | QVQLVQSGAEVKKPGASVKVSCKASGYIFITYWMTWVRQAPGQGLEWMGQI |
| | VH | FPASGSADYNEKFEGRVTMTTDTSTSTAYMELRSLRSDDTAVYYCARGGGGF |
| | | AYWGQGTLVTVSS |
| 84 | tildrakizumab- | DIQMTQSPSSLSASVGDRVTITCRTSENIYSYLAWYQQKPGKAPKLLIYNAKTL |
| | VL | AEGVPSRFSGSGSGTDFTLTISSLOPEDFATYYCQHHYGIPFTFGQGTKVEIK |
| 85 | brazikumab- | QVQLVESGGGVVQPGRSLRLSCAASGFTFSSYGMHWVRQAPGKGLEWVAVI |
| | VH | WYDGSNEYYADSVKGRFTISRDNSKNTLYLQMNSLRAEDTAVYYCARDRGY |
| | | TSSWYPDAFDIWGQGTMVTVSS |
| 86 | brazikumab- | QSVLTQPPSVSGAPGQRVTISCTGSSSNTGAGYDVHWYQQVPGTAPKLLIYGS |
| | VL | GNRPSGVPDRFSGSKSGTSASLAITGLQAEDEADYYCQSYDSSLSGWVFGGGT |
| | | RLTVL |
| 87 | mirikizumab- | QVQLVQSGAEVKKPGSSVKVSCKASGYKFTRYVMHWVRQAPGQGLEWMGY |
| | VH | INPYNDGTNYNEKFKGRVTITADKSTSTAYMELSSLRSEDTAVYYCARNWDT |
| | | GLWGQGTTVTVSS |
| 88 | mirikizumab- | DIQMTQSPSSLSASVGDRVTITCKASDHILKFLTWYQQKPGKAPKLLIYGATSL |
| | VL | ETGVPSRFSGSGSGTDFTLTISSLQPEDFATYYCQMYWSTPFTFGGGTKVEIK |
| 89 | IL4RA ECD | GNMKVLQEPTCVSDYMSISTCEWKMNGPTNCSTELRLLYQLVFLLSEAHTCIP |
| | | ENNGGAGCVCHLLMDDVVSADNYTLDLWAGQQLLWKGSFKPSEHVKPRAPG |
| | | NLTVHTNVSDTLLLTWSNPYPPDNYLYNHLTYAVNIWSENDPADFRIYNVTYL |
| | | EPSLRIAASTLKSGISYRARVRAWAQCYNTTWSEWSPSTKWHNSYREPFEQH |
| 90 | Anti-IL-23- | SEQ ID NO: 110 + SEQ ID NO: 55 + SEQ ID NO: 58 |
| | TGFbRII | |
| | ECD (HC) | |
| 91 | Anti-IL-23- | SEQ ID NO: 110 + SEQ ID NO: 55 + SEQ ID NO: 61 |
| | VEGFR | |
| | (HC) | |
| 92 | Anti-IL-23- | SEQ ID NO: 110 + SEQ ID NO: 55 + SEQ ID NO: 62 |
| | BTLA ECD | |
| | (HC) | |
| 93 | Anti-IL-23- | SEQ ID NO: 110 + SEQ ID NO: 55 + SEQ ID NO: 63 |
| | TIM3 ECD | |
| 94 | Anti-IL-23- | SEQ ID NO: 110 + SEQ ID NO: 55 + SEQ ID NO: 64 |
| | IL 1OR ECD | |
| 95 | Anti-IL-23- | SEQ ID NO: 110 + SEQ ID NO: 55 + SEQ ID NO: 65 |
| | LIGHT | |
| | ECD | |
| 96 | Anti-IL-23- | SEQ ID NO: 110 + SEQ ID NO: 55 + SEQ ID NO: 66 |
| | CD80 ECD | |
| 97 | Anti-IL-23- | SEQ ID NO: 110 + SEQ ID NO: 55 + SEQ ID NO: 67 |
| | CD86 ECD | |
| 98 | Anti-IL-23- | SEQ ID NO: 110 + SEQ ID NO: 55 + SEQ ID NO: 68 |
| | SIRPa ECD | |
| 99 | Anti-IL-23- | SEQ ID NO: 110 + SEQ ID NO: 55 + SEQ ID NO: 69 |
| | CD30L | |
| | ECD | |
| 100 | Anti-IL-23- | SEQ ID NO: 110 + SEQ ID NO: 55 + SEQ ID NO: 70 |
| | 41BBL | |
| | ECD | |
| 101 | Anti-IL-23- | SEQ ID NO: 110 + SEQ ID NO: 55 + SEQ ID NO: 71 |
| | OX40L | |
| | ECD | |
| 102 | Anti-IL-23- | SEQ ID NO: 110 + SEQ ID NO: 55 + SEQ ID NO: 72 |
| | GITRL | |
| | ECD | |
| 103 | Anti-IL-23- | SEQ ID NO: 110 + SEQ ID NO: 55 + SEQ ID NO: 73 |
| | ICOSL ECD | |
| 104 | Anti-IL-23- | SEQ ID NO: 110 + SEQ ID NO: 55 + SEQ ID NO: 74 |
| | CD40L | |
| | ECD | |
| 105 | Anti-IL-23- | SEQ ID NO: 110 + SEQ ID NO: 55 + SEQ ID NO: 75 |
| | TNFR2 | |
| | ECD | |
| 106 | Anti-IL-23- | SEQ ID NO: 110 + SEQ ID NO: 55 + SEQ ID NO: 76 |
| | RANK ECD | |
| 107 | Anti-IL-23- | SEQ ID NO: 110 + SEQ ID NO: 55 + SEQ ID NO: 77 |
| | IL IRA | |
| 108 | Anti-IL-23- | SEQ ID NO: 110 + SEQ ID NO: 55 + SEQ ID NO: 78 |
| | IL22BP | |
| | ECD | |
| 109 | Anti-IL-23- | SEQ ID NO: 110 + SEQ ID NO: 55 + SEQ ID NO: 89 |
| | IL4RA ECD | |
| 110 | guselkumab- | EVQLVQSGAEVKKPGESLKISCKGSGYSFSNYWIGWVRQMPGK |
| | HC | GLEWMGIIDPSNSYTRYSPSFQGQVTISADKSISTAYLQWSSLKA |
| | | SDTAMYYCARWYYKPFDVWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSC |
| | | DKTHTCPPCPAPEAAGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| 111 | IgG1 | ASTKGPSVFPLAPSSKSTSGGTAALGCLVKDYFPEPVTVSWNSG |
| | constant | ALTSGVHTFPAVLQSSGLYSLSSVVTVPSSSLGTQTYICNVNHKP |
| | region | SNTKVDKKVEPKSCDKTHTCPPCPAPEAAGGPSVFLFPPKPKDTL |
| | (LALA) | MISRTPEVTCVVVDVSHEDPEVKFNWYVDGVEVHNAKTKPREE |
| | | QYNSTYRVVSVLTVLHQDWLNGKEYKCKVSNKALPAPIEKTISK |
| | | AKGQPREPQVYTLPPSRDELTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| 112 | IgG1 | ASTKGPSVFPLAPSSKSTSGGTAALGCLVKDYFPEPVTVSWNSG |
| | constant | ALTSGVHTFPAVLQSSGLYSLSSVVTVPSSSLGTQTYICNVNHKP |
| | region (WT) | SNTKVDKKVEPKSCDKTHTCPPCPAPELLGGPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| 113 | IgG1 Fc | EPKSCDKTHTCPPCPAPEAAGGPSVFLFPPKPKDTLMISRTPEVTC |
| | (LALA) | VVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 114 | IgG1 Fc | EPKSCDKTHTCPPCPAPELLGGPSVFLFPPKPKDTLMISRTPEVTC |
| | (WT) | VVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 115 | CTLA4 | MGVLLTQRTLLSLVLALLFPSMASMAMHVAQPAVVLASSRGIAS |
| | ECD | FVCEYASPGKATEVRVTVLRQADSQVTEVCAATYMMGNELTFL |
| | | DDSICTGTSSGNQVNLTIQGLRAMDTGLYICKVELMYPPPYYLGI |
| | | GNGTQIYVIDPEPCPDSDQ |
| 116 | IgG1 Fc | EPKSCDKTHTCPPCPAPELLGGPSVFLFPPKPKDTLMISRTPEVTC |
| | (TfR- | VVVDVSHEDPEVKFNWYVDGVEVHNAKTKPREEQYNSTYRVV |
| | 35.21.16) | SVLTVLHQDWLNGKEYKCKVSNKALPAPIEKTISKAKGQPREPQ |
| | | VYTLPPSREEMTKNQVSLTCLVKGFYPSDIAVEWESYGTEWSSY |
| | | KTTPPVLDSDGSFFLYSKLTVTKEEWQQGFVFSCSVMHEALHNH |
| | | YTQKSLSLSPGK |

### EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57 (b) (1), to relate to each and every individual publication, database entry (e.g., Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

What is claimed is:

1. A recombinant molecule comprising:
a) an interleukin-23 (IL-23) inhibiting polypeptide (IIP), wherein the IIP comprises IL-23 binding polypeptide or an IL-23R binding polypeptide; and
b) a target binding polypeptide moiety that binds one or more immune checkpoint proteins or immune stimulatory receptors.

2. The recombinant molecule of claim 1, wherein the target binding polypeptide binds an immune checkpoint protein as an antagonist or as an agonist.

3. The recombinant molecule of claim 1 or 2, wherein the immune checkpoint protein is a T cell co-inhibitory receptor or ligand or an innate inhibitory receptor or ligand.

4. The recombinant molecule of any one of claims 1-3, wherein the immune checkpoint protein is selected from programmed death-1 (PD1; CD279), programmed death ligand 1 (PDL1), programmed death ligand 2 (PDL2), cytotoxic T-lymphocyte antigen-4 (CTLA4; CD152), B and T lymphocyte attenuator (BTLA), V-domain immunoglobulin suppressor of T cell activation (VISTA), T cell immunoglobulin and ITIM domain (TIGIT), lymphocyte-activation gene 3 (LAG-3; CD223), T-cell immunoglobulin and mucin domain 3 (Tim-3; HAVCR2), carcinoembryonic antigen-related cell-adhesion molecule 1 (CEACAM1), CD47, signal regulatory protein alpha (SIRPa), Major Histocompatibility Complex, Class I, G (HLA-G), Ig-like transcript 2 (ILT2; LILRB1), or Ig-like transcript 4 (ILT4, LILRB2).

5. The recombinant molecule of claim 1, wherein the immune stimulatory receptor is selected from 4-1BB (CD137), Inducible T-cell costimulator (ICOS; CD278), OX-40 (CD134), glucocorticoid-induced TNFR-related protein (GITR; CD357), CD40, Herpesvirus entry mediator (HVEM), CD28, or CD27.

6. The recombinant molecule of any one of claims 1-5, wherein the IIP binds and inhibits IL-23.

7. The recombinant molecule of claim 6, wherein the IIP binds and inhibits the IL-23p19 subunit.

8. The recombinant molecule of any one of claims 1-5, wherein the IIP binds and inhibits IL-23R.

9. The recombinant molecule of any one of claims 1-8, wherein the IIP comprises an antibody or an antigen binding fragment thereof.

10. The recombinant molecule of claim 9, wherein the IIP is an antibody or an antigen binding fragment thereof, wherein the antigen binding fragment thereof comprises a fragment crystallizable (Fc) region, a fragment antigen binding (Fab) region, a single chain variable fragment (scFv), a light chain or a functional portion thereof, a variable region of the light chain (VL), a constant region of the light chain (CL), a heavy chain or a functional portion thereof, a variable region of the heavy chain (VH), a constant region of the heavy chain (CH), at least one complementarity-determining region (CDR) or a portion thereof, or any combination thereof.

11. The recombinant molecule of claim 10, wherein the antibody or an antigen binding fragment thereof comprises a monoclonal antibody that targets the human IL-23p19 subunit.

12. The recombinant molecule of claim 11, wherein the antibody or antigen binding fragment thereof comprises one or more of the six CDRs or an antigen binding portion thereof selected from any one of risankizumab, guselkumab, tildrakizumab, brazikumab, and mirikizumab.

13. The recombinant molecule of claim 12, wherein the antibody or antigen binding fragment thereof is guselkumab.

14. The recombinant molecule of claim 1, wherein the target binding polypeptide comprises an antibody or antigen binding fragment thereof.

15. The recombinant molecule of claim 14, wherein the target binding polypeptide is an antibody or an antigen binding fragment thereof, wherein the antigen binding fragment thereof comprises a fragment crystallizable (Fc) region, a fragment antigen binding (Fab) region, a single chain variable fragment (scFv), a light chain or a functional portion thereof, a variable region of the light chain (VL), a constant region of the light chain (CL), a heavy chain or a functional portion thereof, a variable region of the heavy chain (VH), a constant region of the heavy chain (CH), at least one complementarity-determining region (CDR) or a portion thereof, or any combination thereof.

16. The recombinant molecule of claim 2, wherein the target binding polypeptide binds an immune checkpoint protein as an antagonist, wherein the target binding polypeptide comprises a ligand-binding sequence of the extracellular domain (ECD) of an immune checkpoint protein.

17. The recombinant molecule of claim 16, wherein the ECD of the immune checkpoint protein is capable of specifically binding one or more of its cognate ligands expressed or displayed on a tumor cell or immune cell.

18. The recombinant molecule of claim 17, wherein the immune cell is an antigen presenting cell (APC), myeloid-derived suppressor cell (MDSC), CD4 T cell, or T_{H}17 cell.

19. The recombinant molecule of any one of claims 16-18, wherein the ECD is capable of specifically binding programmed death-1 ligand 1 (PDL1; CD274; B7-H1) and/or programmed death-1 ligand 2 (PDL2).

20. The recombinant molecule of claim 19, wherein the target binding polypeptide comprises the PD1 (CD279) extracellular domain (PD1-ECD) or ligand-binding fragment thereof.

21. The recombinant molecule of any one of claims 1-20, wherein the target binding polypeptide comprises the amino acid sequence of SEQ ID NO: 56, or an amino acid sequence having at least 80% identity to SEQ ID NO: 56 or a ligand-binding fragment thereof.

22. The recombinant molecule of any one of claims 1-20, wherein the target binding polypeptide comprises one or more modifications of the amino acid sequence of SEQ ID NO: 56 or a ligand-binding fragment thereof, wherein the target binding polypeptide comprises substitution, deletion, insertion, or inversion of 1-10 amino acid residues.

23. The recombinant molecule of claim 22, wherein the one or more modifications increase the affinity of the target binding polypeptide to PDL1 or PDL2 or both, compared to the affinity of wild type PD1-ECD to its ligands.

24. The recombinant molecule of claim 22 or 23, wherein the one or more modifications are selected from A132I, S87G, P89L, N116S, G124S, S127V, A140V.

25. The recombinant molecule of claim 24, wherein the modification is A132I.

26. The recombinant molecule of claim 25, wherein the target binding polypeptide has the amino acid sequence of SEQ ID NO: 57.

27. The recombinant molecule of any one of claims 1-26, wherein the IIP is linked to the target binding polypeptide moiety via a linker.

28. The recombinant molecule of claim 27, wherein the target binding polypeptide moiety is linked to the C terminus of the IIP.

29. The recombinant molecule of claim 27 or 28, wherein the linker is selected from a non-cleavable linker, a peptide linker, a flexible linker, a rigid linker, a helical linker, and a non-helical linker.

30. The recombinant molecule of any one of claims 27-29, wherein the linker is a peptide linker having an amino acid sequence comprising (GGGGS)n, wherein n is 1, 2, 3, 4, 5, 6, 7, or 8.

31. The recombinant molecule of claim 30, wherein the linker comprises the amino acid sequence of SEQ ID NO: 55.

32. The recombinant molecule of any one of claims 1-31, wherein the recombinant molecule comprises a first polypeptide having an amino acid sequence of SEQ ID NO: 53 and a second polypeptide having an amino acid sequence of SEQ ID NO: 54.

33. A host comprising the recombinant molecule of any one of claims 1-32.

34. A polynucleotide sequence encoding the recombinant molecule of any one of claims 1-32.

35. A vector comprising the polynucleotide of claim 36.

36. A polypeptide comprising the recombinant molecule of any one of claims 1-32.

37. A pharmaceutical composition comprising the recombinant molecule of any one of claims 1-32 or the vector of claim 35.

38. The pharmaceutical composition of claim 37, further comprising a pharmaceutically acceptable excipient.

39. A method of treating a neoplastic disease, a cancer, or an immune disorder in a subject, comprising administering to the subject an effective amount of a pharmaceutical composition comprising a recombinant molecule of any one of claims 1-32, or the vector of claim 35.

40. The method of claim 39, wherein the subject has cancer.

41. The method of claim 40, wherein the recombinant molecule comprises a first polypeptide having an amino acid sequence of SEQ ID NO: 53 and a second polypeptide having an amino acid sequence of SEQ ID NO: 54.

42. The method of any one of claims 39-41, wherein the cancer is selected from prostate cancer, pancreatic cancer, biliary cancer, colon cancer, rectal cancer, liver cancer, kidney cancer, lung cancer, testicular cancer, breast cancer, ovarian cancer, brain cancer, skin cancer, bladder cancer, and head and neck cancers, melanoma, sarcoma, multiple myeloma, leukemia, and/or lymphoma.

43. The method of any one of claims 39-42, wherein the method suppresses tumor growth for at least 10, 15, or 20 days

44. The method of any one of claims 39-42, wherein the method reduces tumor growth by at least 5%, 10%, 15%, or 20%.

45. The method of any one of claims 39-44, further comprising administering to the subject a therapeutic agent comprising an anti-CTLA4-TGFβRII molecule.

46. The method of any one of claims 39-44, further comprising administering one or more anti-cancer agents.

47. The method of claim 46, wherein the one or more anti-cancer agents comprises an immunotherapeutic agent, chemotherapeutic molecule, antibody, antibody-drug conjugate, small molecule kinase inhibitor, hormonal agent, androgen synthesis inhibitor, androgen receptor antagonist, anti-angiogenic agent, cell therapy, CAR-T cellular therapy, CAR-NK cellular therapy, radionuclide therapy, ionizing radiation, ultraviolet radiation, cryoablation, thermal ablation, a selective estrogen receptor modulator (SERM), a selective estrogen receptor degrader (SERD), or radiofrequency ablation.

48. The method of claim 47, wherein the immunotherapeutic agent is selected from immune checkpoint inhibitor, immune stimulatory receptor agonist, immune stimulatory cytokine/cytokine receptor agonist, immune inhibitory cytokine/cytokine receptor antagonist, tumor vaccine, immunomodulatory imide drug, CAR-T cells, CAR-NK cells, or oncolytic virus.

49. The method of any one of claims 46-48, wherein the administration of the one or more anti-cancer agents reduces or prevents severe immune-related adverse events or toxicity more effectively compared to administration of the one or more anti-cancer agents alone.

50. The method of any one of claims 46-48, wherein the administration of the one or more anti-cancer agents enhances reduction of tumor growth or suppresses tumor growth more effectively compared to administration of the one or more anti-cancer agents alone.

51. The method of any one of claims 39-50, wherein the subject is a mammal.

52. The method any one of claims 39-50, wherein the subject is a human.

53. A method of treating a neoplastic disease or a cancer in a subject, comprising administering to the subject one or more therapeutic agents, wherein the one or more therapeutic agents comprises:
a. a first therapeutic agent comprising an inhibitor of IL-23 signaling; and
b. a second therapeutic agent comprising any one or more of the following:
(i) one or more modulators wherein each is an antagonist of one or more immune checkpoint proteins;
(ii) one or more modulators wherein each is an agonist of one or more immune stimulatory receptors;
(iii) one or more modulators wherein each is an antagonist of the signaling of one or more cytokines;
(iv) one or more modulators wherein each is an agonist of one or more cytokine receptors;
(v) one or more modulators wherein each modulates one or more cell surface molecules expressed or displayed on the cell surface of a tumor cell or an immune cell;
(vi) one or more immune cells comprising CAR-T cells, CAR-NK cells, or hematopoietic stem cells;
(vii) one or more immunogenic chemotherapeutic agents; and/or
(viii) one or modulators wherein each is an antagonist of one or more immune inhibitory enzymes.

54. The method of claim 53, wherein the inhibitor of IL-23 signaling comprises an IL-23 binding moiety that is a recombinant protein that binds IL-23.

55. The method of claim 53, wherein the inhibitor of IL-23 signaling comprises an IL-23R binding moiety that is a recombinant protein that binds IL-23R.

56. The method of claim 53, wherein the inhibitor of IL-23 signaling comprises a recombinant molecule of any one of claims 1-32.

57. The method of claim 54 or 55, wherein the inhibitor of IL-23 signaling is an antibody or an antigen binding fragment thereof, wherein the antigen binding fragment thereof comprises a fragment crystallizable (Fc) region, a fragment antigen binding (Fab) region, a single chain variable fragment (scFv), a light chain or a functional portion thereof, a variable region of the light chain (VL), a constant region of the light chain (CL), a heavy chain or a functional portion thereof, a variable region of the heavy chain (VH), a constant region of the heavy chain (CH), at least one complementarity-determining region (CDR) or an antigen-binding portion thereof, or combinations thereof.

58. The method of claim 57, wherein the antibody or an antigen binding fragment thereof comprises a monoclonal antibody targeting an IL-23 subunit.

59. The method of claim 58, wherein the monoclonal antibody targets the IL-23p19 subunit.

60. The method of claim 58 or 59, wherein the antibody or an antigen binding fragment thereof comprises one or more of the six CDRs or an antigen binding portion thereof selected from any one of risankizumab, guselkumab, tildrakizumab, brazikumab, and mirikizumab.

61. The method of claim 55, wherein the IL-23R binding moiety comprises an antibody or antigen binding fragment thereof.

62. The molecule of claim 61, wherein the antibody or antigen binding fragment thereof comprises one or more of the six CDRs or an antigen binding portion thereof of AS2762900-00.

63. The method of any one of claims 53-62, wherein the second therapeutic agent comprises a modulator that is an antagonist of one or more immune checkpoint proteins.

64. The method of claim 63, wherein the immune checkpoint protein is selected from programmed death-1 (PD1; CD279), programmed death ligand 1 (PDL1), programmed death ligand 2 (PDL2), cytotoxic T-lymphocyte antigen-4 (CTLA4; CD152), B and T lymphocyte attenuator (BTLA), V-domain immunoglobulin suppressor of T cell activation (VISTA), T cell immunoglobulin and ITIM domain (TIGIT), lymphocyte-activation gene 3 (LAG-3; CD223), T-cell immunoglobulin and mucin domain 3 (Tim-3; HAVCR2), carcinoembryonic antigen-related cell-adhesion molecule 1 (CEACAM1), CD47, signal regulatory protein alpha (SIRPa), Major Histocompatibility Complex, Class I, G (HLA-G), Ig-like transcript 2 (ILT2; LILRB1), or Ig-like transcript 4 (ILT4, LILRB2).

65. The method of claim 63 or 64, wherein the second therapeutic agent comprises a modulator that is an antagonist of PD1 signaling.

66. The method of claim 65, wherein the antagonist of PD1 signaling is a polypeptide that targets PD1.

67. The method of claim 65 or 66, wherein the modulator is an inhibitor comprising a monoclonal antibody or an antigen binding fragment thereof targeting PD1 (CD279).

68. The method of claim 67, wherein the antibody or antigen binding fragment thereof comprises one or more of the six CDRs or an antigen binding portion thereof selected from pembrolizumab, nivolumab, cemiplimab, dostarlimab, spartalizumab, camrelizumab, sintilimab, sasanlimab, tiselizumab, or toripalimab.

69. The method of claim 63, wherein the modulator is an inhibitor of the checkpoint protein selected from programmed death-1 ligand 1 (PDL1; CD274; B7-H1), programmed death-1 ligand 2 (PDL2), or both.

70. The method of claim 69, wherein the modulator is a polypeptide that targets PDL1, PDL2, or both.

71. The method of claim 69 or 70, wherein the polypeptide is an antibody or an antigen binding fragment thereof targeting PDL1, PDL2, or both.

72. The method of claim 71, wherein the antibody or an antigen binding fragment thereof comprises one or more of the six CDRs or an antigen binding portion thereof selected from any one of durvalumab, avelumab, or atezolizumab.

73. The method of claim 63, wherein the modulator is a polypeptide inhibitor of the checkpoint protein CTLA-4.

74. The method of claim 73, wherein the polypeptide is an antibody or an antigen binding fragment thereof targeting CTLA-4.

75. The method of claim 73 or 74, wherein the antibody or an antigen binding fragment thereof comprises one or more of the six CDRs or an antigen binding portion thereof selected from any one of ipilimumab or tremelimumab.

76. The method of claim 63, wherein the modulator is a polypeptide inhibitor of the checkpoint protein LAG-3.

77. The method of claim 76, wherein the polypeptide is an antibody or an antigen binding fragment thereof targeting LAG-3.

78. The method of claim 76 or 77, wherein the antibody or an antigen binding fragment thereof comprises one or more of the six CDRs or an antigen binding portion thereof selected from any one of relatlimab, fianlimab, Sym022, GSK2831781, TSR-033, iermilimab, favezelimab, tebotelimab, FS118, or pavunalimab.

79. The method of claim 63, wherein the modulator is a polypeptide inhibitor of the checkpoint protein TIGIT.

80. The method of claim 79, wherein the polypeptide is an antibody or an antigen binding fragment thereof targeting TIGIT.

81. The method of claim 79 or 80, wherein the antibody or an antigen binding fragment thereof comprises one or more of the six CDRs or an antigen binding portion thereof selected from any one of tiragolumab, vibostolimab, BMS-986207, ociperlimab, etigilimab, domvanalimab, EOS-448, SEA-TGT, ASP8374, COM902, or IBI939.

82. The method of any one of claims 53-62, wherein the second therapeutic agent comprises a modulator that is an agonist of one or more immune stimulatory receptors.

83. The method of claim 82, wherein the immune stimulatory receptor is selected from 4-1BB (CD137), Inducible T-cell costimulator (ICOS; CD278), OX-40 (CD134), glucocorticoid-induced TNFR-related protein (GITR; CD357), CD40, Herpesvirus entry mediator (HVEM), CD28, or CD27.

84. The method of claim 83, wherein the second therapeutic agent is a polypeptide that comprises CD40L or a CD40-binding fragment thereof.

85. The method of claim 83 or 84, wherein the second therapeutic agent is a polypeptide that comprises CD80 or CD86; or a CD28-binding fragment thereof.

86. The method of any one of claims 53-62, wherein the second therapeutic agent comprises a modulator of a cell surface molecule expressed or displayed on a tumor cell or tumor-associated stromal cell.

87. The method of claim 86, wherein the cell surface molecule is selected from a growth factor receptor, transforming growth factor-beta receptor (TGFβR), a tumor necrosis factor receptor (TNFR) superfamily receptor, an Ig superfamily receptor, a vascular endothelial growth factor receptor (VEGFR), an epidermal growth factor receptor (EGFR), a platelet-derived growth factor receptor (PDGFR), a tumor cell surface molecule, a cytokine receptor, or a chemokine receptor.

88. The method of any one of claims 53-62, wherein the second therapeutic agent comprises a modulator of a cell surface molecule expressed or displayed on an immune cell.

89. The method of claim 88, wherein the immune cell is a T cell, an NK cell, or a myeloid cell.

90. The method of claim 88 or 89, wherein the cell surface molecule is a tumor necrosis factor receptor (TNFR) superfamily receptor, an Ig superfamily receptor, a cytokine receptor, chemokine receptor, T cell co-stimulatory molecule receptor, a T cell co-inhibitory molecule receptor, or a natural killer (NK) cell receptor.

91. The molecule of any one of claims 88-90, wherein the cell surface molecule is a myeloid cell inhibitory receptor, or a myeloid cell stimulatory receptor.

92. The method of claim 86 or 88, wherein the cell surface receptor is SIRPa or CD47.

93. The method of claim 92, wherein the second therapeutic agent inhibits the binding of SIRPa to CD47.

94. The method of claim 93, wherein the second therapeutic agent is a polypeptide that binds CD47.

95. The method of claim 94, wherein the polypeptide is an antibody or antigen binding fragment thereof targeting CD47.

96. The method of claim 95, wherein the antibody or an antigen binding fragment thereof comprises one or more of the six CDRs or an antigen binding portion thereof of selected from magrolimab, ZL-1201, TJ011133, STI-6643, SRF231, SHR-1603, IMC-002, IBI188, CC-90002, AO-176, or AK117.

97. The method of claim 94, wherein the polypeptide comprises the SIRPa extracellular domain or a CD47-binding fragment thereof.

98. The method of claim 97, wherein the polypeptide is selected from evorpacept, TTI-621, or TTI-622.

99. The method of claim 93, wherein the second therapeutic agent is polypeptide that binds SIRPa.

100. The method of any one of claims 53-62, wherein the second therapeutic agent is an antagonist of the signaling of one or more cytokines.

101. The method of claim 100, wherein the cytokine is transforming growth factor-beta (TGFb).

102. The method of claim 101, wherein the modulator is an inhibitor of TGFb signaling selected from a small molecule kinase inhibitor, polypeptide comprising the TGFbRII ECD or a TGFb-binding fragment thereof, or an antibody or an antigen binding fragment thereof selected from an anti-TGFB antibody, an anti-TGFβR antibody, an anti-GARP antibody, or an anti-LAP antibody.

103. The method of claim 102, wherein the small molecule kinase inhibitor is a TGFβR small molecule kinase inhibitor comprising galunisertib.

104. The method of claim 102, wherein the anti-TGFB antibody or an antigen binding fragment thereof comprises one or more of the six CDRs or an antigen binding portion thereof fresolimumab.

105. The method of claim 102, wherein the polypeptide comprising the TGFbRII ECD or a TGFb-binding fragment thereof is selected from AVID200, bintrafusp alfa (M7824), anti-CTLA4-TGFbRII, SIRPa ECD-TGFbRII, anti-CEA-TGFbRII, anti-PSMA-TGFbRII, anti-IL6R-TGFbRII, anti-PD1-TGFbRII, anti-EGFR-TGFbRII, or anti-HER2-TGFbRII.

106. The method of any one of claims 100-105, wherein the cytokine is selected from one or more of the following: IL-4, IL-13, IL-10, IL-6, IL-1b, IL-17, IL-22, or VEGF.

107. The method of claim 106, wherein the cytokine is IL-4 or IL-13.

108. The method of claim 106 or 107, wherein the modulator is a polypeptide that targets IL4 receptor alpha (IL4Ra).

109. The method of claim 108, wherein the polypeptide is an antibody or antigen-binding fragment that comprises one or more of the six CDRs or an antigen binding portion thereof of dupilumab.

110. The method of claim 106, wherein the cytokine is IL1b.

111. The method of claim 110, wherein the second therapeutic agent comprises anakinra.

112. The method of claim 110, wherein the second therapeutic agent comprises one or more of the six CDRs or an antigen binding portion thereof of canakinumab.

113. The method of claim 106, wherein the cytokine is IL10.

114. The method of claim 113, wherein the second therapeutic agent comprises an IL10-binding sequence of the extracellular domain of IL 10R, or an antibody or antigen-binding fragment thereof that targets IL 10 or IL 10R.

115. The method of any one of claims 53-62, wherein the second therapeutic agent is an agonist of one or more cytokine receptors.

116. The method of claim 115, wherein the cytokine receptor is selected from IL 12R, IL15R, and IL18R.

117. The method of claim 115 or 116, wherein the second therapeutic agent is a polypeptide that comprises IL-12.

118. The method of claim 88, wherein the cell surface molecule is a tumor cell surface molecule selected from CA125, CA19-9, CD30, carcinoembryonic antigen-related cell adhesion molecule 1 (CEACAM1), CEACAM5 or cluster of differentiation 66e (CD66e), CEACAM6, DLL3, DLL4, DPEP3, EGFR EGFRvIII, GD2, HER2, HER3, HGF, IGF1R, IL13Ra2, LIV-1, LRRC15, MUC1, PRLR, PSCA, PSMA, PTK7, SEZ6, SLAMF7, TF, cMet, claudin, mesothelin, nectin4, uPAR, GPNMB, CD79b, CD22, NaPi2b, SLTRK6, STEAP1, MUC16, CD37, GCC, AGC-16, 5T4, CD70, TROP2, CD74, CD27L, Fra, CD138, CA6, CD38, SLAMF7, BCMA, CD20, CD19, CD33, or CD30.

119. The method of claim 118, wherein the modulator is an inhibitor comprising a monoclonal antibody or an antigen binding fragment thereof targeting the tumor cell surface molecule.

120. The method of claim 119, wherein the antibody or an antigen fragment thereof comprises one or more of the six CDRs or an antigen binding portion thereof selected from any one of labetuzumab, cergutuzumab, cetuximab, necitumumab, panitumumab, depatuxizumab, trastuzumab, pertuzumab, enfortumab, or sacituzumab.

121. The method of claim 88, wherein the immune cell comprises an antigen presenting cell (APC), a myeloid-derived suppressor cell (MDSC), a dendritic cell, a natural killer cell, or a macrophage.

122. The method of claim 88 or 121, wherein the immune cell comprises, a T_{H}17 cell, a CD4 T cell, a CD8 T cell, a Treg cell, gamma delta T cell, NK cell, innate lymphoid cell (ILC), or gamma delta T17 cell.

123. The method of any one of claims 53-122, wherein treatment with the combination of the first and second agents reduces or suppresses tumor growth, prevents or reduces severe immune-related adverse events, increases overall survival or progression-free survival, reduces or prevents adverse events or reduced toxicity, or reduces or prevents bone metastases or skeletal-related adverse events, more effectively than treatment with the second agent alone.

124. The method of any one of claims 53-123, wherein the cancer is selected from prostate cancer, pancreatic cancer, biliary cancer, colon cancer, rectal cancer, liver cancer, kidney cancer, lung cancer, testicular cancer, breast cancer, ovarian cancer, brain cancer, skin cancer, bladder cancer, and head and neck cancers, melanoma, sarcoma, multiple myeloma, leukemia, and/or lymphoma.

125. The method of any one of claims 53-124, further comprising administering to the subject a therapeutic agent comprising an anti-CTLA4-TGFβRII molecule.

126. The method of any one of claims 53-124, further comprising administering one or more anti-cancer therapies.

127. The method of claim 126, wherein the one or more anti-cancer therapies comprise a immunotherapeutic agent, chemotherapeutic molecule, antibody, antibody-drug conjugate, small molecule kinase inhibitor, hormonal agent, androgen synthesis inhibitor, androgen receptor antagonist, anti-angiogenic agent, cell therapy, CAR-T cellular therapy, CAR-NK cellular therapy, radionuclide therapy, ionizing radiation, ultraviolet radiation, cryoablation, thermal ablation, a selective estrogen receptor modulator (SERM), a selective estrogen receptor degrader (SERD), or radiofrequency ablation.

128. The method of claim 127, wherein the immunotherapeutic agent is selected from immune checkpoint inhibitor, immune stimulatory receptor agonist, immune stimulatory cytokine/cytokine receptor agonist, immune inhibitory cytokine/cytokine receptor antagonist, tumor vaccine, immunomodulatory imide drug, CAR-T cells, CAR-NK cells, or oncolytic virus.

129. The method of any one of claims 53-128, wherein the subject is a mammal.

130. The method of any one of claims 53-128, wherein the subject is a human.
